(19) United States
(12) Patent Application Publication
(10) Pub. No.: US 2024/0254565 A1
(43) Pub. Date: Aug. 1, 2024

(54) UNIQUE CANCER ASSOCIATED FIBROBLAST SUBSETS PREDICT RESPONSE TO IMMUNOTHERAPY

(71) Applicants: VANDERBILT UNIVERSITY, Nashville, TN (US); THE TRUSTEES OF COLUMBIA UNIVERSITY IN THE CITY OF NEW YORK, New York, NY (US)

(72) Inventors: Michael J. Korrer, Nashville, TN (US); Young J. KIM, Nashville, TN (US); Andrea CALIFANO, New York, NY (US)

(21) Appl. No.: 18/565,854

(22) PCT Filed: Jun. 21, 2022
(86) PCT No.: PCT/US2022/073056

§ 371 Date: Nov. 30, 2023

Related U.S. Application Data
(60) Provisional application No. 63/212,927, filed on Jun. 21, 2021.

(51) Int. Cl. C12Q 1/6886; A61K 35/17; A61K 39/00; C07K 16/28; C12Q 1/6809

(52) U.S. Cl. CPC C12Q 1/6886; A61K 35/17; A61K 39/4611; A61K 39/4631; C07K 16/2818; C07K 16/2827; C12Q 1/6809; C12Q 2600/158

(57) ABSTRACT

Disclosed herein is a method for treating a solid tumor in a subject that involves detecting in a tumor biopsy sample from the subject enrichment of a cancer associated fibroblast (CAF) subset disclosed herein and then treating the subject with an immunotherapy. Also discussed is a method for treating a solid tumor in a subject that involves isolating cancer associated fibroblasts (CAFs) from the subject, isolating and expanding the disclosed subset of CAFs, and administering the expanded CAF subset to the subject in combination with an immunotherapy.

### CROSS-REFERENCE TO RELATED APPLICATIONS

This application claims benefit of U.S. Provisional Application No. 63/212,927, filed Jun. 21, 2021, which is hereby incorporated herein by reference in its entirety.

### BACKGROUND

Use of PD-1/PD-L1 immune checkpoint inhibitors (ICI) is currently the first line therapy for recurrent/metastatic head and neck squamous cell carcinoma. Yet, overall response rates can be as low as 20%, with increased responses in tumors with elevated PD-L1 expression. The factors guiding resistance mechanisms to ICI remain largely unknown, making it difficult to predict who will respond and who will not. Accordingly, there remains an unmet need for reliable biomarkers predictive of response to guide patient selection and optimization of ICI treatment.

### SUMMARY

Disclosed herein is a method for treating a solid tumor in a subject that involves detecting in a tumor biopsy sample from the subject enrichment of a cancer associated fibroblast (CAF) subset disclosed herein and then treating the subject with an immunotherapy. In some embodiments, the CAF subset comprises at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% of the fibroblasts in the tumor biopsy.

Also discussed is a method for treating a solid tumor in a subject that involves isolating cancer associated fibroblasts (CAFs) from the subject, isolating and expanding the disclosed subset of CAFs, and administering the expanded CAF subset to the subject in combination with an immunotherapy.

In some embodiments, the CAF subset is identified by a plurality of genes in a first cluster (Cluster-0) selected from the group consisting of AEBP1, ALPL, ANGPTL2, ANKH, ANKRD28, ANTXR1, APOL2, APP, ASPN, B4GALT1, BAMBI, BGN, BICC1, BNIP3, C10orf10, C1orf54, CADM1, CCDC3, CCNG2, CD276, CD9, CDC42EP3, CDH11, CDKN2A, CERCAM, CITED2, CKAP4, CKB, CLEC11A, CLMP, CNKSR3, COL12A1, COL16A1, COL1A1, COL1A2, COL3A1, COL5A1, COL5A2, COL6A3, COL8A1, COPZ2, CPE, CSF1, CTGF, CTHRC1, CTNNB1, CTSK, CTSZ, CXCL12, CXCL14, CYR61, DCN, DDIT4, DLX5, EDIL3, EFEMP2, EFNA5, EMILIN1, ENAH, ENPP2, ERRF11, EVI2A, FAM134B, FAP, FBLN1, FBLN7, FCGRT, FGFR1, FKBP10, FLRT2, FMOD, FNDC3B, FSCN1, FXYD6, GADD45G, GALNT1, GAS1, GGT5, GJA1, GLT8D2, GOLIM4, GOLM1, GPNMB, HAPLN1, HERPUD1, HES1, HSPG2, HTRA1, ID2, IGF2, IGFBP4, IGHG1, IGHG3, IGHG4, IGKC, IGLC2, IRX3, JCHAIN, KCNQ1OT1, KCTD12, KDELR3, LAMB1, LAPTM4A, LAYN, LIMCH1, LMO7, LOXL1, LPAR1, LSP1, LUM, MAFB, MAP4K4, MARCKS, MDK, MFAP2, MIR99AHG, MMP11, MMP14, MMP2, MT1E, MT1X, MT-ND3, MT-ND4, MT-ND5, MXRA5, MXRA8, MZB1, NBL1, NNMT, NPC2, NR3C1, NRP2, OLFML2B, OLFML3, P3H4, PALLD, PCOLCE, PDGFC, PDGFD, PDPN, PFN2, PIK3R1, PLD3, PLEKHA5, PODNL1, POSTN, PPA1, PRRX1, PRSS23, PSAP, PTPRS, QKI, RAB31, RA114, RCAN1, RCN3, RGS3, S100A10, S100A13, S100A4, SCRG1, SDC1, SDC2, SEPP1, SERPINF1, SERPINH1, SERTAD4, SESN3, SFRP2, SH3BP5, SMOC2, SNAI2, SPARC, SRP14, SSPN, SSR4, STK17B, THBS2, TIMP3, TPST1, TSC22D3, UNC5B, VCAM1, VCAN, VIM, WIPF1, YPEL2, ZEB1, ZFHX4, and ZFP36L2.

In some embodiments, the CAF subset is identified by a plurality of genes in a second cluster (Cluster-3) selected from the group consisting of ACTB, ADAM121, ADAM19, ADAMTS2, AKR1B11, ALDH2, ANPEP, ANXA2, ANXA5, ANXA61, APOL1, AQP1, ARID5A2, ARL4C, BASP11, BMP2, BPGM, BST21, C12orf75, CALD11, CCL21, CD2481, CD82, CEP57L1, CHMP1B1, CHPF2, COL15A1, COL1A11, COL3A11, COL5A11, COL5A21, COL5A31, COL6A1, COL6A2, COL6A31, COL7A1, CRABP2, CREB3L21, CTHRC11, CXCL11, CXCL3, CXCL81, DIXDC1, DNAJA11, EIF5A, EMILIN11, ENQ1, EVA1A, F2R1, F3, FAM129B, FAM19A5, FARP11, FKBP11, FN1, FSTL11, FTH11, GAPDH, GBP1, GCNT1, GLIPR2, GLIS31, GNAI2, GOLM11, GPM6B, GREM11, GUCY1A31, H1F01, HES41, HIF1A1, HILPDA, HLA-A1, HLA-B, HLA-C, HSPA181, HSPA5, HSPH1, HTRA3, IFI271, IFI44L1, IFI61, IFIT1, IFIT31, IFITM1, IGFBP3, IL11, IL24, IL32, IL61, INHBA, IRF7, ISG151, ISLR, ITGA5, ITGAV1, KLF6, LAMA41, LGALS1, LGMN1, LINC001521, LOXL11, LOXL2, LY6E, MAGEH1, MEST, METRNL, MFAP21, MFAP51, MME1, MMP1, MMP111, MMP141, MT-CO2, MX1, MX21, NES1, NOX4, NREP, NTM1, OAS1, PAPPA1, PDLIM4, PFN1, PHLDA31, PKM, PLAT, PLAU, PLXNC1, PMEPA1, POSTN1, PRRX21, PTGES1, PTK7, PXDN, RARRES2, RIN2, RORA1, RP11-115D19.1, RP3-325F22.5, S100A11, S100A16, S100A61, SAT1, SCG5, SCX, SEC23A, SELM, SERINC2, SERPINE1, SERPNH11, SGK1, SPON2, STC2, STEAP11, SUGCT, SULF1, TAGLN1, TCTN3, TGFBI, TGM2, TIMP2, TMEM158, TMEM45A, TMSB10, TNC, TNFAIP61, TNFRSF12A1, TPM11, TPM21, TRIOBP, TUBA1A, TUBA1C, TWIST21, TYMP, VCAN1, WARS, WDFY1, WIPI11, WNT5A1, XAF1, ZFAND2A, and ZMPSTE24.

In some embodiments, the CAF subset is detected by detecting differential expression of at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 181, 182, 183, 184, 185, 186, 187, 188, 189, or 190 genes in the first cluster; at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 171, 172, 173, 174, 175, 176, 177, 178, 179, 180, 181, or 182 genes in the second cluster; or a combination thereof.

In some embodiments, the CAF subset is identified by a plurality of differentially activated proteins in a first cluster (Cluster-0) selected from the group consisting of ABCC9, ACAP1, ACKR1, ADAP2, ADGRL4, ADRA2B, AGT, AK1, AKAP13, AKNA, ALPL, ANKH, ANTXR1, ANXA1, ANXA4, APBB1IP, APOLD1, APP, ARF4, ARF5, ARHGAP15, ARHGAP30, ARHGEF19, ARID4B, ARID5B, ARL2BP, ARL4A, ARL4C, ARRDC2, ASH1L, ATF6B, ATP2B1, ATP6AP2, ATRAID, AXL, BASP1, BATF, BCL11B, BCL2L11, BTG1, BTG2, C18orf32, C2orf88, C3AR1, CADM1, CALML5, CAMLG, CAPN2, CAPNS2, CAPS, CBLB, CCL4L2, CCL5, CCNH, CCR6, CCR7, CD1C, CD2, CD24, CD247, CD27, CD3D, CD3E, CD3G, CD48, CD5, CD53, CD7, CD74, CD8A, CD8B, CD9, CD96, CD99, CDC42BPA, CDC42EP3, CDH11, CDKN2A, CDKN2B, CEACAM6, CEBPB, CERCAM, CFB, CHD9, CLEC10A, CLEC4A, CLMP, CLU, CNIH1, CNKSR3, COLEC12, CORO1A, CPE, CPM, CPNE7, CRABP2, CREB3L1, CREB3L2, CREM, CRYAB, CSDE1, CSF2RA, CTLA4, CTNNB1, CTSH, CTSZ, CXCR3, CXCR4, CXCR6, CYBRD1, DAP, DAPP1, DDAH2, DDIT4, DDR2, DDX5, DERL3, DLX5, DNAJB6, DPYSL3, DSG1, DST, DTHD1, DUSP1, DUSP2, DUSP4, EBF1, EEF1D, EID1, EIF5, ELF1, EMP1, EMP2, EMP3, ENAH, ENPP2, EPB41, EPB41L2, EPHB2, ESD, ETS1, ETV3, EVI2A, EVL, EZR, F2RL3, FAP, FBXW7, FCER1A, FCER1G, FCGR2B, FGF7, FGFR1, FGFR2, FHL1, FNBP1, FNDC4, FOSL2, FOXC1, FOXC2, FOXO3, FPR1, FXYD6, FYN, FZD1, GAS1, GATA2, GDI2, GJA1, GLIPR1, GNAS, GNG7, GPBP1, GPC1, GPR132, GPR157, GPR171, GPR183, GPR65, GRAP2, GRIN2A, GSN, GSPT1, GZMA, GZMB, HCST, HDAC7, HDLBP, HERPUD1, HEXIM1, HLA-B, HLA-DOA, HLA-DOB, HLA-DPA1, HLA-DPB1, HLA-DQA1, HLA-DQB1, HLA-DRA, HLA-DRB1, HLA-DRB5, HLA-E, HNRNPDL, HOXB2, HPGD, HSPG2, HYAL2, ICOS, ID2, ID3, IFI16, IFITM2, IFNG, IKZF1, IKZF3, IL10RA, IL2RA, IL2RB, IL2RG, IL6ST, INPP5D, INTS6, ITGA10, ITGA11, ITGA4, ITGB1BP1, ITGB2, ITGB5, ITGBL1, ITM2B, ITM2C, JADE1, JAML, JMJD1C, JUN, JUNB, KCNMA1, KIR2DL4, KLF2, KLF9, KLRB1, KLRC1, KLRC2, KLRD1, LAX1, LBH, LCK, LCP1, LGALS3, LIFR, LPAR2, LPL, LRP1, LSP1, LTB, LY86, LY96, MAFB, MAGED1, MAPK13, MARCO, MARVELD1, MEF2C, MEOX2, MGST3, MKX, MMP2, MORF4L1, MS4A6A, MTUS1, MYADM, MYC, NDFIP1, NDN, NDRG2, NEO1, NET1, NFIX, NOTCH2, NOTCH4, NR3C1, NR4A3, NRP2, NSG1, NTRK3, NUDT4, PALLD, PASK, PBX1, PDCD1, PDCD4, PDE4B, PDGFC, PDGFRA, PEBP1, PFDN5, PIK31P1, PIK3R1, PINK1, PITX1, PLEKHO1, PLSCR4, PNRC1, PPIC, PPP1R10, PPP1R2, PPP2R5C, PRDX4, PRF1, PRMT2, PRRX1, PRSS27, PSCA, PSIP1, PTGIS, PTH1R, PTN, PTP4A2, PTPRC, PTPRD, RAB30, RAB31, RAB32, RAB33A, RABAC1, RAMP2, RAMP3, RAP1B, RASGEF1B, RASGRP1, RASSF4, RASSF5, RASSF7, RBM38, RBPJ, REL, RERG, RGL4, RGS1, RGS10, RGS3, RHOD, RHOF, RHOH, RHOJ, RIPK2, RORA, RPS27L, RPSA, RRBP1, RUNX2, RUNX3, S100A10, S100A6, S1PR3, S1PR4, SAP18, SDC1, SDC2, SDCBP, SELP, SEMA7A, SERBP1, SFPQ, SH2D1A, SH2D2A, SH3BGRL, SH3BP5, SH3KBP1, SIT1, SKAP1, SKAP2, SKIL, SLA, SLA2, SLC29A1, SLC38A5, SLC41A2, SLC44A1, SLC7A5, SLCO2A1, SMAP2, SMOC1, SMOC2, SOCS1, SOD1, SOX18, SP100, SPIB, SPOCK2, SPRED1, SRSF2, STAB1, STEAP4, STK17B, STK4, STX11, STXBP6, SVIP, SYTL3, TANK, TBC1D10C, TCEA3, TCEAL2, TCEAL4, TERF2IP, TGFB1I1, TGFBI, THY1, TLE4, TMEM204, TMEM59, TNFAIP8L3, TNFRSF18, TNFSF12, TOB1, TRAF1, TRAT1, TRIM22, TSC22D3, TSHZ2, TSPAN4, TSPO, TWIST1, TXNIP, TYROBP, UBC, UBE2B, UNC5C, UTRN, VAMP2, VASN, VASP, VCAM1, VOPP1, VPS37B, WASF2, WSB1, WWTR1, YWHAQ, ZBTB16, ZBTB20, ZEB1, ZFAND5, ZFHX3, ZFHX4, ZFP36L2, ZNF106, ZNF296, and ZNF428.

In some embodiments, the CAF subset is identified by a plurality of differentially activated proteins in a second cluster (Cluster-3) selected from the group consisting of ABL2, ACAP11, ACHE1, ACKR3, ACKR41, ACTB1, ACTG1, ACTN11, ADAM12, ADD3, ADM1, ADRB21, AES1, AGPAT21, AGTRAP1, AHNAK21, AKAP131, ANGPT21, ANGPTL41, ANKRD112, ANKRD12, ANTXR11, ANXA5, AP2B11, AP2M1, AP2S1, AP3S1, APBA2, APOE1, AQP91, ARC1, ARF11, ARF41, ARHGAP152, ARNTL21, ARPC21, ASH1L1, ATP1B1, ATP2B11, ATP6AP21, ATP6V1G11, AVPR1A1, AXL1, B2M1, B4GALT1, BASP11, BATF1, BATF3, BAX1, BDKRB11, BHLHE401, BIRC31, BMP21, BSG1, BST21, BZW11, C18orf321, C31, C5AR11, CALD11, CALM2, CALM3, CALR1, CAP11, CASP11, CAV11, CAV21, CBFA2T31, CBX31, CCL21, CCL4L21, CCL8, CCR62, CCRL21, CD1641, CD1771, CD1E1, CD2741, CD300E1, CD402, CD441, CD471, CD51, CD591, CD63, CD79A2, CD82, CD831, CDC421, CDC42SE1, CEMIP, CERCAM1, CFL11, CFLAR, CHD31, CHIC2, CHMP4A, CHMP51, CHN11, CHP1, CHST11, CKS2, CLEC2B1, CLEC5A, CLEC7A1, CLIC11, CLIC4, CNIH41, CPNE11, CRABP21, CREB3L11, CSF2RA1, CSF2RB1, CSNK2B2, CTNNAL11, CXCL101, CXCL21, CXCL3, CYBA, DDR21, DIO21, DLL11, DNAJB61, DPP4, DRAP11, DUSP61, ECM1, EDF1, EDNRA1, EDNRB1, EFNA1, EHD11, EID3, EMP11, ENG1, ENO11, ENTPD11, ENY2, EREG1, ERO1A1, ETS22, ETV31, EVA1A, F2R, F2RL21, F2RL31, F31, FAP1, FCER1A2, FCER1G1, FCGR1B2, FKBP8, FLNA1, FLT31, FOSL21, FOXP1, FOXS11, FPR12, FRMD61, FST1, FSTL11, GABARAP, GAPDH1, GEM1, GGT51, GLIPR11, GNAI1, GNB11, GNG11, GNG2, GNG51, GPBP11, GPM6B1, GPR1571, GPR1831, GPX11, GREM1, GRK5, GRN1, GYPC1, HBEGF1, HCAR22, HCAR32, HES42, HIF1A, HINT11, HIVEP3, HLA-A1, HLA-B1, HLA-C1, HLA-F1, HM13, HPGD1, HRAS, HSBP11, HSPA51, ICAM11, IFI271, IFI61, IFITM11, IFITM21, IFITM31, IFNGR11, IGF2, IGFBP31, IGFBP61, IGFLR11, IL10, IL11, IL15RA1, IL1A1, IL1B1, IL1R1, IL1R22, IL1RAP1, IL2RA1, IL2RG1, IL61, IL7R, ILK1, INHBA, INSIG11, IRF41, IRF7, ITGA1, ITGA5, ITGAV, ITGB1, ITGB42, ITGB61, ITSN22, KCNJ81, KCNK61, KIR2DL41, KLF61, KLRB11, KLRC11, LAMP5, LCP21, LGALS1, LGALS3BP1, LGALS9, LHX81, LILRA11, LILRB21, LIMA11, LIMS11, LMCD1, LMO41, LOXL2, LPXN, LRRFIP11, LSR2, LTB1, LY6E1, LY6K, LYPD11, MAP1B1, MAP3K81, MARCKS1, MARCKSL1, MGST21, MIF1, MMP14, MORF4L2, MSC, MSN1, MSX2, MX1, MXD11, MYADM2, MYH9, MYO101, MYO1G1, NACC1, NAMPT1, NCOA71, NDUFA131, NEDD41, NEDD81, NEO11, NET12, NFE2L3, NFKB11, NGFR1, NLRP31, NME21, NOTCH31, NRG11, NRP11, NTM1, OLR11, PAG1, PALLD1, PARK72, PARP14, PDGFRB1, PDIA31, PDIA61, PDLIM11, PDPN, PFDN51, PFN11, PHLDA1, PILRA1, PIM21, PKIG1, PKM1, PLAT1, PLAU, PLAUR, PLEC2, PLEK1, PLK2, PLP22, PLPP32, PLPP4, PLSCR11, PLXDC11, PMAIP1, PMEPA1, POLR2L1, PON2, PPIC1, PPP1R21, PRCP1, PRDM1, PRDX41, PRKAR1A1, PRMT11, PROCR2, PRRX11, PRRX2, PSMA4, PTEN, PTGER3, PTGES, PTGIR, PTHLH, PTK7, PTPN11, PTPRE1, PTTG11, RAB101, RAB131, RAB1A, RAB301, RAB311, RAB321, RAB33A1, RAB5C1, RABAC11, RANBP11, RAP1A, RAP1B1, RASD1, RASGEF1B1, RBPJ1, RBPMS, REL2, RGL41, RGS161, RGS2, RGS31, RGS4, RHEB2, RHOBTB11, RHOC, RHOF1, RHOG2, RHOH1, RIN2, RIPK21, RND31, RPS27L1, RRAD1, RRBP11, S100A111, S100A121, S100A16, S100A61, SCAND11, SCG5, SDC11, SDC41, SECTM12, SELE1, SERPINB9, SERPINE12, SGIP11, SGK11, SHISA51, SIGLEC102, SKIL1, SLA21, SLC1A51, SLC24A41, SLC2A31, SLC2A6, SLC39A14, SLC3A21, SLC41A21, SLC7A111, SLC7A51, SLC9A3R22, SNAI21, SOD21, SOX11, SOX4, SQSTM11, STAT11, STAT2, STEAP1, STEAP21, STX111, SUB11, SULF1, SULF21, SYTL21, TANK1, TAX1BP31, TCF41, TFP11, TGFB1I11, TGFB31, TGIF1, THBD1, THBS11, THY11, TLR21, TMEM2041, TNF1, TNFAIP31, TNFAIP61, TNFRSF12A1, TNFRSF1A, TNFRSF1B1, TNFRSF21, TNFSF101, TNFSF13B2, TNFSF141, TRAF11, TREM11, TRIB11, TRIM221, TSC22D11, TSPAN151, TSPAN9, TWIST11, TWIST2, TXNDC171, UACA1, UBB1, UBE2B1, UBE2D31, UBE2L31, VAMP21, VAMP51, VAMP82, VASP2, VCAM11, VDAC11, VEGFA1, WNT2, WNT5A, XBP11, YWHAH1, ZNF2671, ZNF2962, ZNF469, ZNF503, and ZNHIT11.

In some embodiments, the CAF subset is detected by detecting at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 200, 210, 220, 230, 240, 250, 260, 270, 280, 290, 300, 310, 320, 330, 340, 350, 360, 370, 380, 390, 400, 410, 420, 430, 440, 441, 442, 443, 444, 445, 456, 447, 448, or 449 differentially activated proteins of in the first cluster; at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 200, 210, 220, 230, 240, 250, 260, 270, 280, 290, 300, 310, 320, 330, 340, 350, 360, 370, 380, 390, 400, 410, 420, 430, 440, 441, 442, 443, 444, 445, 456, 447, 448, 449, 450, 451, 452, 453, 454, 455, 456, 457, 458, 459, 460, 461, 462, 463, 464, 465, 466, 467, 468, 469, 470, 471, or 472 differentially activated proteins in the second cluster; or a combination thereof.

In some embodiments, the immunotherapy is a T cell immunotherapy, such as a chimeric antigen receptor (CAR) T-cell therapy or tumor-infiltrating lymphocyte (TIL) therapy. In some embodiments, the immunotherapy is a checkpoint inhibitor, such as an anti-PD-1 antibody, anti-PD-L1 antibody, anti-CTLA-4 antibody, or a combination thereof.

In some embodiments, the solid tumor is a sarcoma, carcinoma, or lymphoma. In some embodiments, the solid tumor is a melanoma, ovarian, breast, or colorectal cancer

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1D show longitudinal single cell transcriptomic profiles of HNSCC show diverse immune infiltrate and changes associated with immunotherapy. FIG. 1A shows 2-dimensional UMAP Projection of Cells across all samples, processed by VIPER and clustered by resolution-optimized Louvain. Cells are colored by unsupervised cluster number, with fibroblast clusters (4, 6, 7, 9) further labelled by cell type. FIG. 1B is a boxplot of population frequency at baseline and following αPD1 immunotherapy for each cell type cluster in A. CAFs increasing in response to immunotherapy (p<0.01) are circled in blue. FIG. 1C is a heatmap of top 5 most differentially upregulated proteins per cluster for each cell population in FIG. 1A. FIG. 1D shows SingleR cell type inference projected on UMAP plot. Each cluster is assigned a lineage cell type based on its majority SingleR-inferred label.

FIGS. 2A to 2D show fibroblast sub-clustering reveals distinct populations with differential response to αPD1 and association with clinical outcome. FIG. 2A shows 2-dimensional UMAP projection of Cancer-Associated Fibroblasts across all samples, re-clustered by resolution-optimized Louvain and colored by cluster identity. FIG. 2B is a boxplot of cluster frequencies pre vs post nivolumab therapy, such that HNCAF-0 and HNCAF-3 show statistically significant increase in frequency (p<0.01) while HNCAF-1 and HNCAF-2 show significant decrease (p<0.01). FIG. 2C is a heatmap of top 10 most differentially upregulated proteins per cluster for each CAF population. FIG. 1D contains Gene Set Enrichment plots of single-cell gene population markers for each HNCAF cluster (Table 1) in bulk-RNASeq signature of immunotherapy responders vs non-responders, profiled pre-treatment. HNCAF-0 and HNCAF-3 gene sets are significantly enriched in treatment responders (p=3.2×10⁻⁷ and p=1.7×10⁻⁶, respectively).

FIGS. 3A to 3F show HNCAF sub-population states by scRNA-Seq are distinct from CAF phenotypes defined by flow in prior literature, and spatially co-segregate with CD8 T-cells. FIG. 3A shows relative frequencies across patients of Stromal (CD45−Epca−CD31−), Epithelial/Endothelial (CD45−Epcam+/CD31+) and Immune (CD45+) tumor components. FIG. 3B shows a Flow Cytometry gating strategy to isolate CAF phenotypes previously described in the literature, implemented as described in Costa et. al. FIG. 3C shows Relative Frequency for each patient of CAF subtypes from B among total CAFs by flow cytometry. FIG. 3D shows Phenotype-Matching between unsupervised clusters from single-cell RNA-Seq and bulk-RNASeq of sorted populations CAF-S1 to CAF-S4, as well as iCAF and myCAF, from Elyada et. al. Each single-cell population is labelled as the sorted population with highest gene set enrichment. FIG. 3E is a Kaplan-Meier plot of HNCAF-0 Gene Set Enrichment among TCGA head and neck squamous cell carcinoma patients in association with overall survival time. Enrichment scores were binarized by log-rank maximization to “high HNCAF-0” and “low HNCAF-0” and show significant association with improved survival (p=0.014, median survival time=602 days vs 1671 days). FIG. 3F is a Kaplan-Meier plot of HNCAF-1 Gene Set Enrichment among TCGA head and neck squamous cell carcinoma patients in association with overall survival time, as in FIG. 3E. HNCAF-1 enrichment shows significant association with worse survival (p=0.011, median survival time=1718 days vs 773 days).

FIGS. 4A to 4H show HNCAF-0 is associated with better prognosis in TCGA and functionally abrogates T-cell exhaustion in co-culture experiments. FIG. 4A shows pre-treatment DSP immunofluorescence imaging from representative patient treated with αPD1 immunotherapy, such that tumor cell localization is indicated by panCK staining (green), CD8 T-cell localization is indicated by CD8 staining (red), fibroblast localization is indicated by αSMA staining (yellow), and nucleated cells are indicated by DAPI staining (blue). Arrows indicate interactions between αSMA+ fibroblasts and CD8+ T cells. FIG. 4B shows a co-culture experiment of Peripheral Blood Mononuclear Cells (PBMCs) with isolated HNCAF-0 cells, showing reduced T-cell exhaustion (% PD-1+ TIM-3+ cells), increased tissue localization markers (% CD103+ NKG2A+ cells), and increased cytotoxicity (% Perforin+ GzmB+ cells). * indicates p<0.05, ** indicates p<0.01, and *** indicates p<0.001. FIG. 4C shows a co-culture experiment of PBMCs with HNCAF-0 cells in contact-isolating transwell culture, showing reduced T-cell exhaustion and reduced tissue localization markers, but no significant difference in cytotoxicity. FIG. 4D shows co-culture experiment of Tumor-Infiltrating Lymphocytes (TILs) with sorted HNCAF-0 cells, showing increased tissue localization markers and increased cytotoxicity. FIG. 4E shows interferon gamma levels in co-culture of PBMCs and TILs with HNCAF-0 cells, showing significant increase in co-culture with TILs but not PBMCs. FIG. 4F shows a rescue experiment of T-cell co-culture with TGFβ and with or without HNCAF-0. T-cell exhaustion markers on the left plot (% PD-1+ TIM-3+ cells) are rescued by HNCAF-0, and tissue localization markers on the right plot (% CD103+ cells) increase with TGFβ but are unaffected by addition of HNCAF-0. FIG. 4G shows spatial enrichment of HNCAF-0 gene set vs enrichment of T-cell exhaustion signature in Nanostring DSP of tissue slices across patients. No statistically significant association in spatial co-enrichment. FIG. 4H shows spatial enrichment of HNCAF-1 gene set vs enrichment of T-cell exhaustion signature in Nanostring DSP of tissue slices across patients. Signatures are positively correlated with respect to spatial co-localization (correlation coefficient=0.94, p=0.0014).

FIGS. 5A to 5E show single-Cell RNA-Sequencing and VIPER Inference Shows Increased T-cell Activity Induced by Nivolumab. FIG. 5A shows 2-dimensional UMAP projection of single-cell RNA-Seq gene expression data before VIPER is applied, colored by unsupervised cluster grouping. FIG. 5B shows 2-dimensional UMAP projection of gene expression data from FIG. 5C, colored by cell type inferred from SingleR, as in FIG. 1D. FIG. 5C shows T-cell Activity Score Enrichment among T-cell populations pre vs post immunotherapy. FIG. 5D shows Interferon Gamma VIPER-inferred protein activity among T-cell populations pre vs post immunotherapy. FIG. 5E is a plot of inferred receptor-ligand interactions between cell types, such that receptor-ligand pairs with known interaction have significant upregulation of ligand gene expression among fibroblasts, and significant upregulation of corresponding receptor protein activity by VIPER in another cell type. Width of lines is weighted by the number of inferred interactions between fibroblasts (in the middle), and each other cell type.

FIG. 6 shows a flow gating strategy for cell sorting of CAF-S1 through S4 populations.

FIGS. 7A to 7V show Phenotypic Matching of Single-Cell HNCAF Populations to Flow-Sorted Populations from Literature. FIGS. 7A to 7T show Pairwise Gene Set Enrichment of single-cell HNCAF-0 (FIGS. 7A-7D), HNCAF-1 (FIGS. 7E-7H), HNCAF-2 (FIGS. 7I-7L), HNCAF-3 (FIGS. 7M-7P), OR HNCAF-4 (FIGS. 7Q-7T) gene sets among bulk RNA-Seq of sorted CAF populations CAF-S1 (FIGS. 7A, 7E, 7I, 7M, 7Q), CAF-S2 (FIGS. 7B, 7F, 7J, 7N, 7R), CAF-S3 (FIGS. 7C, 7G, 7K, 7O, 7S), and CAF-S4 (FIGS. 7D, 7H, 7L, 7P, 7T), as defined by Costa et. al. Best match by gene set enrichment for each HNCAF cluster is outlined in red. FIGS. 7U and 7V shows Cell-by-Cell enrichment of published iCAF (FIG. 7U) and myCAF (FIG. 7V) gene sets from Elyada et. al. in our single-cell HNCAF dataset, grouped by cluster, such that HNCAF-1 is enriched for iCAF gene set, and HNCAF-2 is enriched for myCAF gene set.

FIGS. 8A and 8B show sorted CAFs in co-culture experiments are strongly enriched for HNCAF-0 and HNCAF-3. FIG. 8A contains Gene Set Enrichment plots of single-cell gene population markers for each HNCAF cluster in bulk-RNASeq of sorted CAFs used for co-culture experiments. HNCAF-0 and HNCAF-3 gene sets are significantly enriched in treatment responders (p=4.2×10⁻² and p=2.1×10⁻², respectively), and no other HNCAF signatures show statistically significant gene set enrichment. FIG. 8B shows CIBERSORTx inference of cell type abundances in bulk-RNASeq of sorted CAFs used for co-culture experiments, across three technical replicates, such that HNCAF-0 and HNCAF-3 constitute the majority of inferred cell frequency. For reference, in single-cell RNASeq (FIG. 2) HNCAF-0 represents 43% of overall CAF frequency, HNCAF-1 represents 22%, HNCAF-2 represents 20%, HNCAF-3 represents 13%, and HNCAF-4 represents 2%.

FIGS. 9A and 9B show HNCAF-0 enrichment is highly specific to Head and Neck Squamous Cell Carcinoma. FIG. 9A is a boxplot of HNCAF-0 gene set enrichment among TCGA tumor types with high stromal involvement. FIG. 9B is a boxplot of HNCAF-1 gene set enrichment among TCGA tumor types with high stromal involvement. LIHC: Liver Hepatocellular Carcinoma, CHOL: Cholangiocarcinoma, BRCA: Breast Cancer, UCS: Uterine Carcinosarcoma, SARC: Sarcoma, PAAD: Pancreatic Adenocarcinoma, HNSC: Head and Neck Squamous Cell Carcinoma.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### CAF Therapy

Disclosed herein is a method for treating a solid tumor in a subject that involves detecting in a tumor biopsy sample from the subject enrichment of a cancer associated fibroblast (CAF) subset disclosed herein and then treating the subject with an immunotherapy.

Also disclosed is a method for treating a solid tumor in a subject that involves isolating cancer associated fibroblasts (CAFs) from the subject, isolating and expanding the disclosed subset of CAFs, and administering the expanded CAF subset to the subject in combination with an immunotherapy.

The disclosed therapeutic compositions may be administered either alone, or as a pharmaceutical composition in combination with diluents and/or with other components such as IL-2, IL-15, or other cytokines or cell populations. Briefly, pharmaceutical compositions may comprise agents or cell populations as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat the cancer. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the CAR-TIL cells described herein may be administered at a dosage of 10⁴ to 10⁹ cells/kg body weight, such as 10⁵ to 10⁶ cells/kg body weight, including all integer values within those ranges. CAR-TIL cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

### Cancers

The cancer treated by the disclosed compositions and methods can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the vulva, chronic lymphocytic leukemia, chronic myeloid cancer, cervical cancer, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, peritoneum, omentum, and mesentery cancer, pharynx cancer, prostate cancer, rectal cancer, renal cancer, skin cancer, soft tissue cancer, testicular cancer, thyroid cancer, ureter cancer, urinary bladder cancer, and digestive tract cancer such as, e.g., esophageal cancer, gastric cancer, pancreatic cancer, stomach cancer, small intestine cancer, gastrointestinal carcinoid tumor, cancer of the oral cavity, colorectal cancer, and hepatobiliary cancer.

The cancer can be a recurrent cancer. Preferably, the cancer is a solid cancer. Preferably, the cancer is melanoma, ovarian, breast and colorectal cancer, even more preferred is melanoma, in particular metastatic melanoma.

### Immunotherapy

In some embodiments, the immunotherapy is a chimeric antigen receptor (CAR) T cell containing CAR polypeptides. A CAR polypeptide is generally made up of three domains: an ectodomain, a transmembrane domain, and an endodomain. The ectodomain is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR). CAR polypeptides generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41 BB, and ICOS, that are able to enhance T-cell activation by T-cell receptors.

Additional CAR constructs are described, for example, in Fresnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD123, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the antigen recognition domain is single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the antigen recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

CAR-T cells involve immune effector cells that are engineered to express CAR polypeptides. These cells are preferably obtained from the subject to be treated (i.e. are autologous). However, in some embodiments, immune effector cell lines or donor effector cells (allogeneic) are used. In still other embodiments, the immune effect cells are not HLA-matched. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some aspects, the disclosed methods involve treating the subject with Adoptive Cell Transfer (ACT) of lymphocytes, such as tumor-infiltrating lymphocytes (TILs), such as HLA-matched TILs.

Tumor-infiltrating lymphocyte (TIL) production is a 2-step process: 1) the pre-REP (Rapid Expansion) stage where you the grow the cells in standard lab media such as RPMI and treat the TILs w/reagents such as irradiated feeder cells, and anti-CD3 antibodies to achieve the desired effect; and 2) the REP stage where you expand the TILs in a large enough culture amount for treating the patients. The REP stage requires cGMP grade reagents and 30-40 L of culture medium. However, the pre-REP stage can utilize lab grade reagents (under the assumption that the lab grade reagents get diluted out during the REP stage), making it easier to incorporate alternative strategies for improving TIL production. Therefore, in some embodiments, the disclosed TLR agonist and/or peptide or peptidomimetics can be included in the culture medium during the pre-REP stage.

Adoptive cell transfer (ACT) is a very effective form of immunotherapy and involves the transfer of immune cells with antitumor activity into cancer patients. ACT is a treatment approach that involves the identification, in vitro, of lymphocytes with antitumor activity, the in vitro expansion of these cells to large numbers and their infusion into the cancer-bearing host. Lymphocytes used for adoptive transfer can be derived from the stroma of resected tumors (tumor infiltrating lymphocytes or TILS). They can also be derived or from blood if they are genetically engineered to express antitumor T cell receptors (TCRs) or chimeric antigen receptors (CARs), enriched with mixed lymphocyte tumor cell cultures (MLTCs), or cloned using autologous antigen presenting cells and tumor derived peptides. ACT in which the lymphocytes originate from the cancer-bearing host to be infused is termed autologous ACT. US 2011/0052530 relates to a method for performing adoptive cell therapy to promote cancer regression, primarily for treatment of patients suffering from metastatic melanoma, which is incorporated by reference in its entirety for these methods.

ACT may be performed by (i) obtaining autologous lymphocytes from a mammal, (ii) culturing the autologous lymphocytes to produce expanded lymphocytes, and (ii) administering the expanded lymphocytes to the mammal. Preferably, the lymphocytes are tumor-derived, i.e. they are TILs, and are isolated from the mammal to be treated, i.e. autologous transfer.

Autologous ACT as described herein may also be performed by (i) culturing autologous lymphocytes to produce expanded lymphocytes; (ii) administering nonmyeloablative lymphodepleting chemotherapy to the mammal; and (iii) after administering nonmyeloablative lymphodepleting chemotherapy, administering the expanded lymphocytes to the mammal.

Autologous TILs may be obtained from the stroma of resected tumors. Tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase).

Expansion of lymphocytes, including tumor-infiltrating lymphocytes, such as T cells can be accomplished by any of a number of methods as are known in the art. For example, T cells can be rapidly expanded using non-specific T-cell receptor stimulation in the presence of feeder lymphocytes and interleukin-2 (IL-2), IL-7, IL-15, IL-21, or combinations thereof. The non-specific T-cell receptor stimulus can e.g. include around 30 ng/ml of OKT3, a mouse monoclonal anti-CD3 antibody (available from Ortho-McNeil®, Raritan, N.J. or Miltenyi Biotec, Bergisch Gladbach, Germany). Alternatively, T cells can be rapidly expanded by stimulation of peripheral blood mononuclear cells (PBMC) in vitro with one or more antigens (including antigenic portions thereof, such as epitope(s), or a cell of the cancer, which can be optionally expressed from a vector, such as an human leukocyte antigen A2 (HLA-A2) binding peptide, e.g., approximately 0.3 μM MART-1: 26-35 (27 L) or gp100:209-217 (210M)), in the presence of a T-cell growth factor, such as around 200-400 μl/ml, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

In some embodiments, nonmyeloablative lymphodepleting chemotherapy is administered to the mammal prior to administering to the mammal the expanded tumor-infiltrating lymphocytes. The purpose of lymphodepletion is to make room for the infused lymphocytes, in particular by eliminating regulatory T cells and other non-specific T cells which compete for homeostatic cytokines Nonmyeloablative lymphodepleting chemotherapy can be any suitable such therapy, which can be administered by any suitable route known to a person of skill. The nonmyeloablative lymphodepleting chemotherapy can comprise, for example, the administration of cyclophosphamide and fludarabine, particularly if the cancer is melanoma, which can be metastatic. A preferred route of administering cyclophosphamide and fludarabine is intravenously. Likewise, any suitable dose of cyclophosphamide and fludarabine can be administered. Preferably, around 40-80 mg/kg, such as around 60 mg/kg of cyclophosphamide is administered for approximately two days after which around 15-35 mg/m2, such as around 25 mg/m2 fludarabine is administered for around five days, particularly if the cancer is melanoma.

Specific tumor reactivity of the expanded TILs can be tested by any method known in the art, e.g., by measuring cytokine release (e.g., interferon-gamma) following co-culture with tumor cells. In one embodiment, the autologous ACT method comprises enriching cultured TILs for CD8+ T cells prior to rapid expansion of the cells. Following culture of the TILs in IL-2, the T cells are depleted of CD4+ cells and enriched for CD8+ cells using, for example, a CD8 microbead separation (e.g., using a CliniMACS<plus>CD8 microbead system (Miltenyi Biotec)). In an embodiment of the method, a T-cell growth factor that promotes the growth and activation of the autologous T cells is administered to the mammal either concomitantly with the autologous T cells or subsequently to the autologous T cells. The T-cell growth factor can be any suitable growth factor that promotes the growth and activation of the autologous T-cells. Examples of suitable T-cell growth factors include interleukin (IL)-2, IL-7, IL-15, IL-12 and IL-21, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. IL-12 is a preferred T-cell growth factor.

Preferably, expanded lymphocytes produced by these methods are administered as an intra-arterial or intravenous infusion, which preferably lasts about 30 to about 60 minutes. Other examples of routes of administration include intraperitoneal, intrathecal and intralymphatic. Likewise, any suitable dose of lymphocytes can be administered. In one embodiment, about 1×10¹⁰ lymphocytes to about 15×10¹⁰ lymphocytes are administered.

The disclosed methods can involve treating the subject with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MED14736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

### Combinations

The disclosed methods can involve treating the subject with a combination of additional therapeutic agents. In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec ST1571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-Ia from the human CXC and C-C chemokine families. Suitable cytokines include cytokine derivatives, cytokine variants, cytokine fragments, and cytokine fusion proteins.

In some embodiments, a therapeutic agent may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the subject further receives radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Cancer Associated Fibroblast Subpopulations Mediate Clinical Immunotherapy Response in Head and Neck Squamous Cell Carcinoma

### Introduction

In human breast cancer, four CAF subtypes, referred to as CAF-S1 to S4, were identified by flow cytometry based on the expression of six fibroblast markers—including fibroblast activation protein (FAP), integrin P1 (CD29), α-smooth muscle actin (α-SMA), fibroblast-specific protein-1 (FSP-1), platelet-derived growth factor receptor β (PDGFRβ), and caveolin-1 (CAV1).

In contrast, only two molecularly and phenotypically distinct CAF subpopulations were identified in pancreatic cancer, based on spatial location and imputed function, as defined by cytokine expression. These include inflammatory CAF (iCAF) and myofibroblastic CAF (myCAF). To assess whether CAF-related or other TME subpopulations may regulate clinical responses to nivolumab, single-cell RNA-sequencing (scRNA-Seq) was leveraged to longitudinally profile human head and neck squamous cell carcinoma, before and after treatment with αPD1 inhibitors. This helped isolate fibroblast subpopulations and define clinically relevant CAF sub-phenotypes in HNSCC at a higher resolution than previous classification schemes. This bioinformatic approach used the VIPER algorithm to address limitations imposed by high noise and gene dropout rates in scRNA-Seq data. Specifically, VIPER leverages knowledge of regulatory networks to allow full quantitative characterization of protein activity, by assessing the enrichment of their transcriptional targets in differentially expressed genes, akin to a highly multiplexed gene reporter assay. On average, the resulting protein activity profiles outperform antibody-based measurements and dramatically outperform gene expression-based analyses in terms of identifying and characterizing molecularly distinct TME subpopulations, thus enabling mechanistic dissection of the HNSCC micro-environment at hitherto unattained resolution. This Example presents the results of these protein activity-based analysis as a complete atlas of the human HNSCC immune and stromal micro-environment.

### Results

Single-Cell Transcriptional Analysis Identifies CAF Populations in the HNSCC Micro-Environment, and their Proteomic Master Regulators that is Associated with Clinical Response to Nivolumab

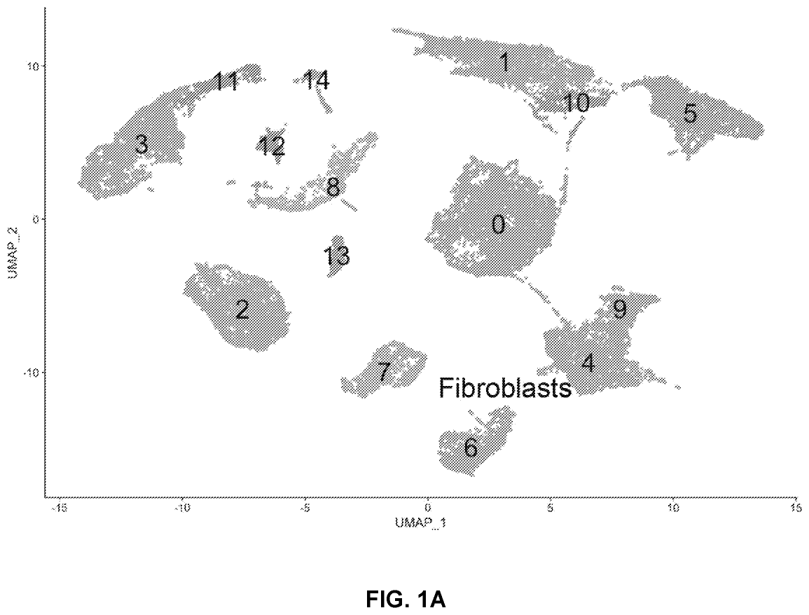

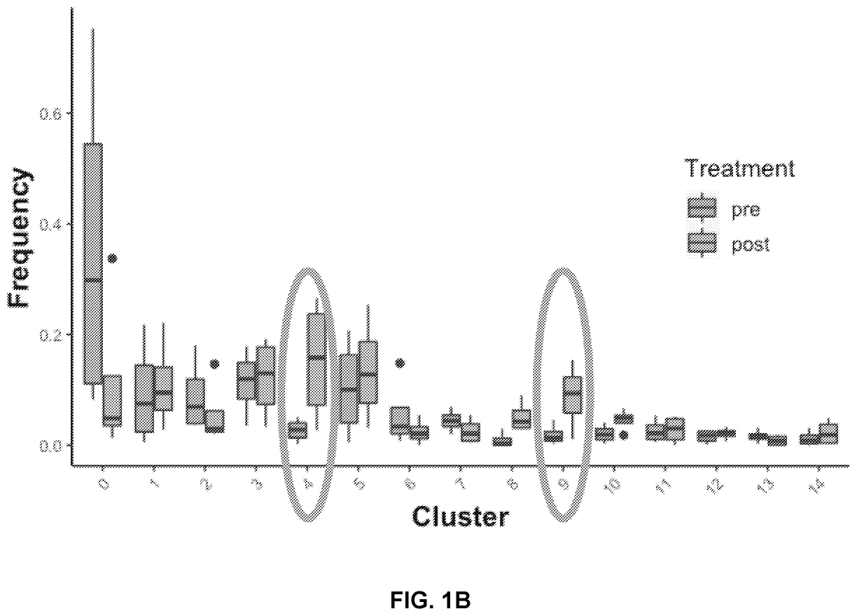

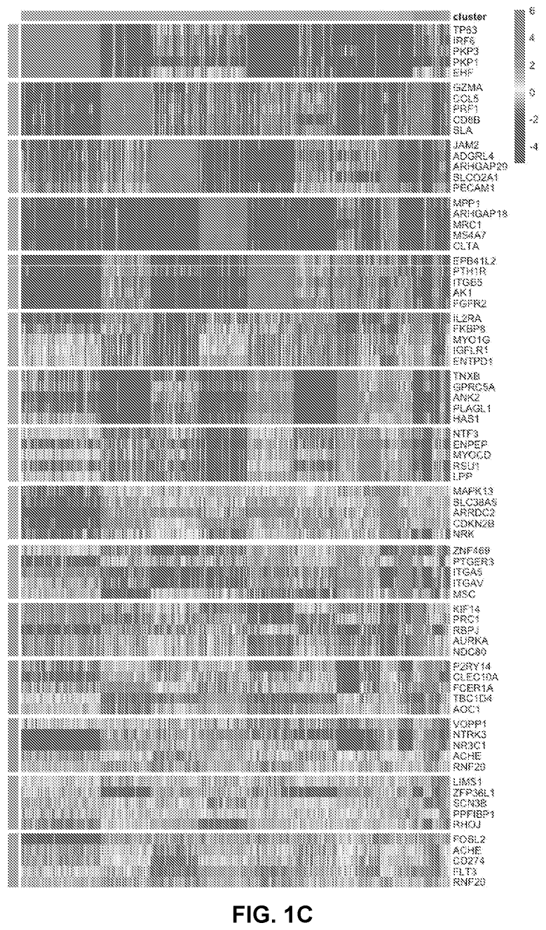

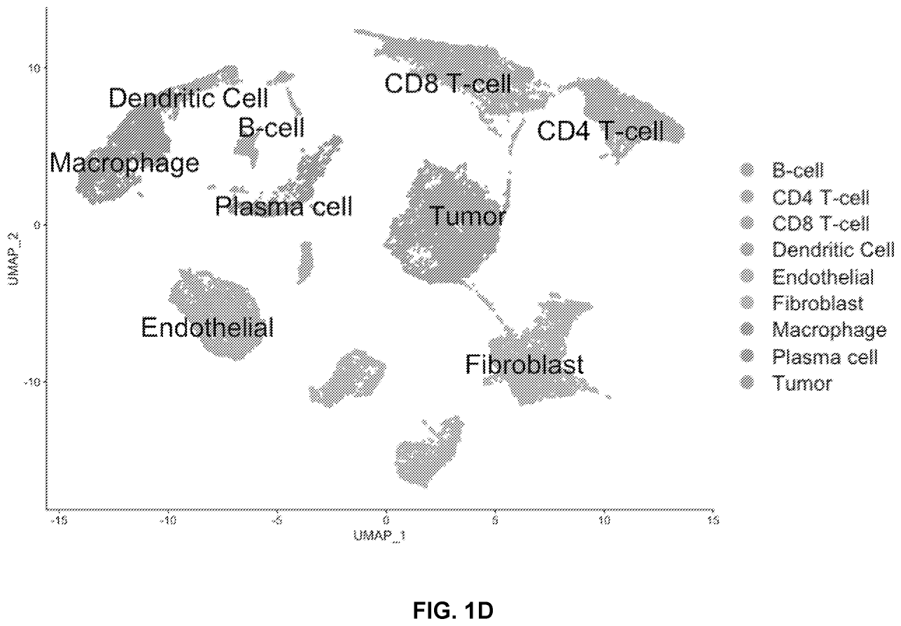

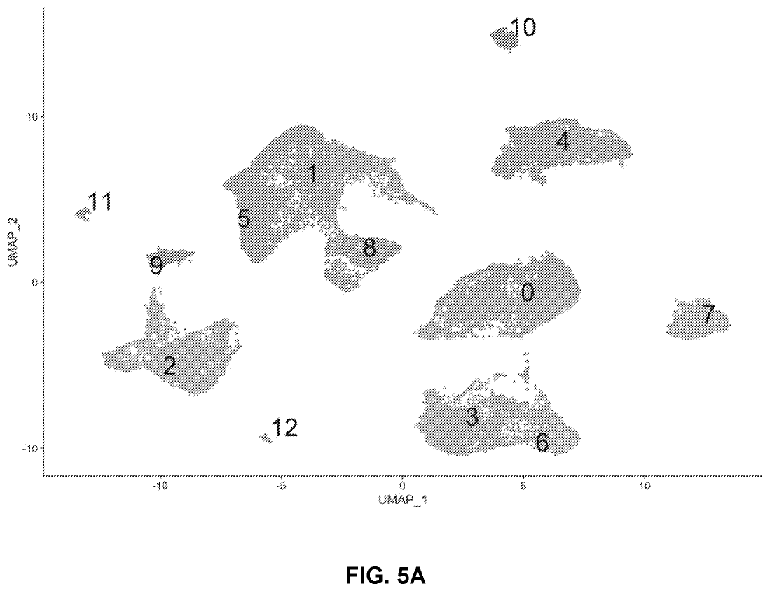

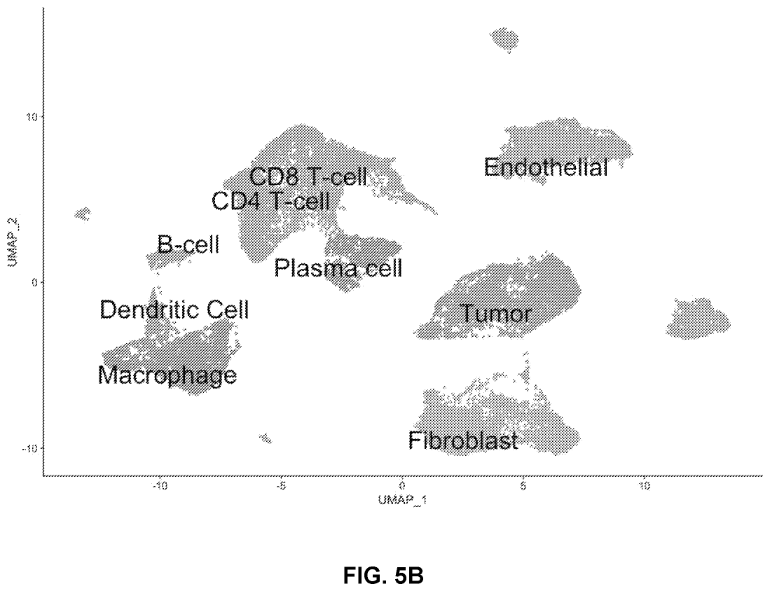

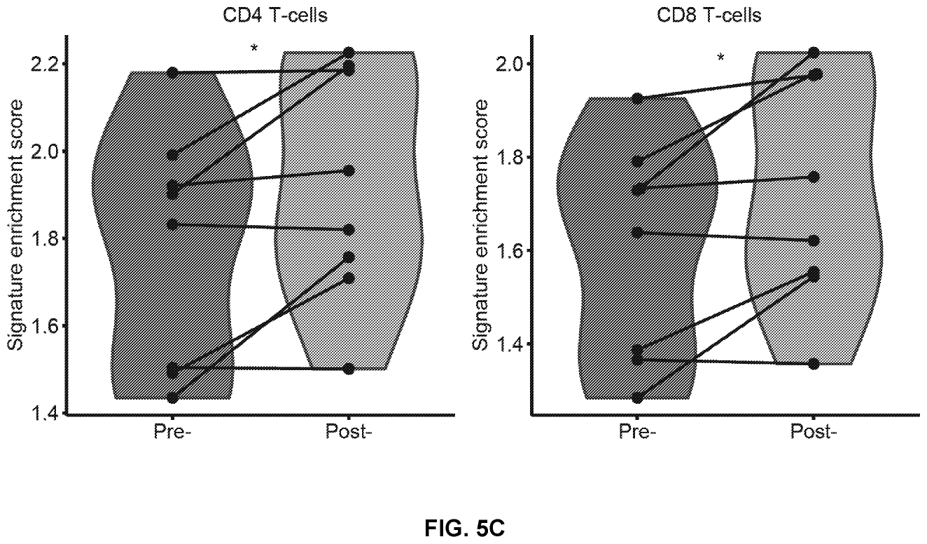

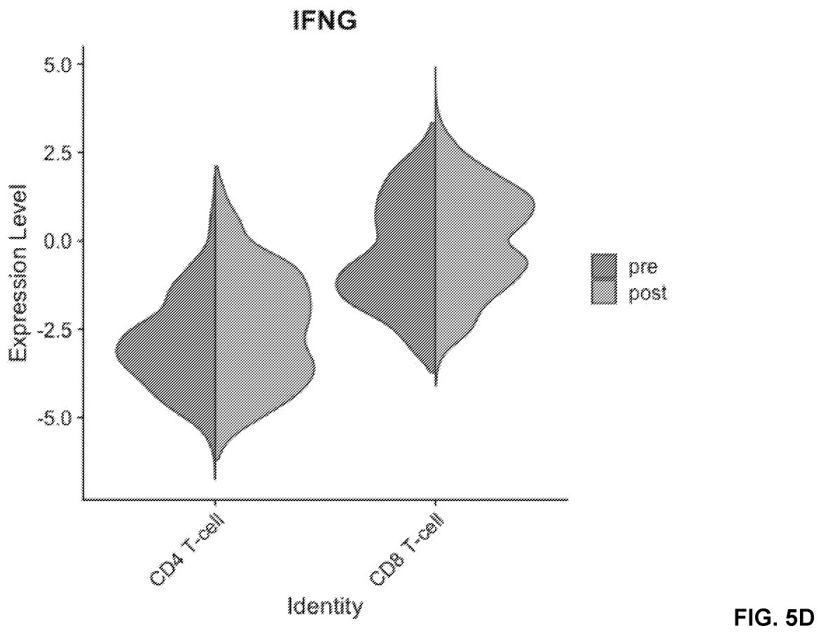

Longitudinal scRNA-Seq of patient tumors, pre and post nivolumab treatment, and gene expression clustering with Seurat revealed 12 broadly-distinct cellular populations, consistently across all four patients (FIG. 5A-5B). To achieve finer-grain cellular subpopulations characterization, scRNA-Seq profiles from each cluster to infer gene regulatory networks were used and the regulatory network-based protein activity inference algorithm, Virtual Inference of Protein-activity by Enriched Regulon (VIPER) was employed. Protein activity-based re-clustering with VIPER identified two additional clusters for a total of 14 distinct cell populations (FIG. 1A). In order to visualize the differences between all the cell populations, we generated a heat map for the top five VIPER-inferred proteins most differentially upregulated in each cluster (FIG. 1C). The full list of differentially active proteins and differentially expressed genes are tabulated in Table 1. Before further analyzing these cell populations, the ability of transcriptomic sequencing to accurately capture treatment-induced changes was assessed. Both gene expression and VIPER analyses revealed increased T cell activity, following nivolumab treatment. This corresponded to greater enrichment of the T-cell activation signature in differentially expressed genes and upregulation of interferon-gamma protein activity, following immunotherapy treatment (FIG. 5C-5D). The fraction of cells in the T-cell cluster also tended to increase with immunotherapy (FIG. 1B). the differential abundance of each cell population pre- and post-treatment was then interrogated. This revealed two clusters presenting highly statistically significant post-treatment cellular fraction increase (FIG. 1B). Cell lineage inference, using SingleR, identified both clusters as fibroblasts, suggesting that PD-1 targeted immunotherapy in head and neck cancer was associated with CAF upregulation (FIG. 1D). Accordingly, imputed receptor-ligand interactions between cell types suggested strong interplay between CAFs and CD8 T cells (FIG. 5E). However, two additional clusters, also characterized as fibroblasts by SingleR, did not show significant fractional representation differences following immunotherapy, thus suggesting the existence of functionally distinct CAF subpopulations within the human TME upon pressure that can reverse T cell-based immunosuppression.

TABLE 1
Differentially Expressed Genes for each cell type cluster shown in FIG. 1A

Cluster 0:
PERP, FXYD3, SFN, KRT6A, S100A14, FAM83A, PDZK1IP1, KRT5, CLDN7,
KRT6B, DMKN, KRT17, DSP, S100A2, SLC2A1, CALML3, PKP1, KLK10, GSTP1,
NDRG1, SAA1, FGFBP1, PTHLH, APOL1, TACSTD2, SPRR1B, KRT14, JUP,
FABP5, KRT15, CSTA, LYPD3, ELF3, KLK8, SDCBP2, SERPINB3, EIF4EBP1,
DSC3, SDC1, SERPINB5, PYGB, GJB2, PITX1, IRF6, PRSS8, MAL2, LY6D,
ATP1B1, SDR16C5, SPRR2D, KRT19, C19orf33, RHOV, LAMB3, KRT16, CTSV,
KRT8, DSC2, LDHA, ERO1A, RP11-54H7.4, GJB6, TRIM29, LGALS7B, RHOD,
PI3, GPX2, COL17A1, AQP3, LY6K, SDC4, PVRL4, HSPB1, PVRL1, RAB25,
DEFB1, DSG3, CLDN1, TUBA1C, KLF5, PKP3, WARS, CLCA2, LAMC2, RBP1,
SERPINB4, VSNL1, RAB38, SLC6A8, SERINC2, DDR1, DHCR24, AHNAK2,
CKMT1B, TNFSF10, SERPINB13, PLPP2, TP63, CXADR, SPINT1, P3H2, KLC3,
LGALS7, TMEM40, LAD1, WDR72, EIF5A, EPPK1, SLPI, ANXA8, CENPW,
CH17-360D5.2, CCNB1, CDH1, F3, S100A16, ANXA2, SOX15, KRT31, CDH3,
NEFL, MMP7, C12orf75, S100A11, TSPAN1, BARX2, HCAR2, TXNDC17,
SPINT2, CDC20, CRB3, CD82, UNC5B-AS1, WDR66, PROM2, ITGB4, ALDH3B2,
EHF, PTK6, DENND2C, ESRP1, CASC9, BBOX1-AS1, CTSC, SNHG25, ITGA3,
ANXA3, AHNAK, FAM213A, ANXA1, BNC1, ECM1, MPZL2, CTNND1, AHCY,
CXCL14, IGFBP6, DSG2, FERMT1, ST14, ITGB6, S100A8, FAM46B, HRASLS2,
OAS1, UBE2C, SOX9, TNS4, C6orf132, LRRC8A, S100A9, ENO1, LY6E,
IGF2BP2, FOSL1, PRNP, NXN, ITGB8, LAMA3, LTBR, HMGA1, KRT18, BIK,
RPL35, CA12, GYLTL1B, FLRT3, SOWAHC, PHLDA2, CKS1B, NRG1, PTTG1,
SCD, PRKDC, PBK, MT1X, NQO1, ADM, ADRB2, BAIAP2L1, MGST1,
SERPINB1, TK1, RAC3, CCDC51, CKB, CA2, TXN, TMSB10, TNNT1, DCBLD1,
GAPDH, MT1G, NEBL, PHLDB2, H2AFZ, RRAD, RND3, IFI27, MAD2L1, PKM,
INHBA, PCNA, EFNB1, MT2A, PHLDA3, ATP2A2, APOL2, ARL4D, CD109,
OCIAD2, PTGFRN, FSTL3, CITED4, NDFIP2, GPRC5A, IFITM1, RTKN,
KIAA0101, PDLIM4, MT1M, LTB4R, CD59, TNFRSF12A, CAV1, IFI6, ITGA6,
NET1, S100A10, SLC15A3, GM2A, MT1E, PRPF6, EFNA1, TPX2, SERPINB2,
HCAR3, CD24, LPCAT4, RARRES3, PPL, DAAM1, CXCL11, KRT75, TMEM238,
CKS2, ZWINT, H3F3B, BIRC5, MALAT1, CDT1, ALDH3A1, CDKN3, MT-CO2,
ENDOD1, EGLN3, CCNB2, MSMO1, SAMD9, MX1, GLTP, SLC3A2, NR1D1,
ISG15, CDK1, IL1RN, HBEGF, CENPK, SLC7A5, IL18, MALL, FST, FAM129B,
CXCL10, ASB2, IRX3, ATP1B3, PLAU, EPHA2, CCDC14, ADAM9, TRIB1,
HSP90B1, UPP1, KRT10, DST, MT-CO3, HMGB1, CDK6, RPS29, SNAPC1,
ANGPTL4, VAV3, NUSAP1, NCOA7, GADD45A, DYNLRB1, IFIT3, CALR, BNIP3,
HSPA5, OASL, C4orf3, ITGA5, CSTB, PDIA6, TMEM45A, ARL6IP1, KPNA2,
NUPR1, SLC38A2, C1orf56, UBE2S, UBC, UGCG, EMP1, and VMP1.
Cluster 1:
CMC1, TUBA4A, GZMH, GZMK, CST7, CD8B, CRTAM, CCL5, PARP8, NKG7,
CD8A, KLRD1, GZMM, RUNX3, SLA2, PRF1, APOBEC3G, CTSW, TRGC2,
CLEC2B, SYTL3, TRAT1, CNOT6L, PTPRC, PTPN22, PIK3R1, SH2D2A,
PPP2R5C, CXCR4, GNLY, PTPN7, CD96, GZMB, ELF1, AC092580.4, GZMA,
ACAP1, ZFP36L2, MCL1, RP11-138A9.1, YPEL5, SLA, FYN, BTG1, EVL, DUSP2,
SH2D1A, RBM38, GPR65, CD3G, IL2RG, CREM, LAG3, GPR171, LEPROTL1,
CD3E, ALOX5AP, SAMSN1, CYTIP, CD3D, DUSP4, CD52, TSC22D3, STK4,
CD69, RPS27, RGS1, CD7, HCST, TRBC2, TRBC1, ISG20, ARHGDIB, CD53,
CCL4, CD2, CORO1A, RPS291, TRAC, SARAF, RNF125, CLEC2D, LCK, LITAF,
CXCR6, PDE3B, VPS37B, JAML, RGCC, FAM129A, AC016831.7, FAM46C,
SMAP2, AKNA, RP11-138A9.2, S1PR4, CD6, RNF19A, PYHIN1, ADGRE5,
TBC1D10C, PDE4D, LDLRAD4, PDCD4, SAMD3, ETS1, CDC42SE2, ARAP2,
RAC2, RBPJ, LINC-PINT, CCNH, SPOCK2, CBLB, KLRC4, EMB, RP11-347P5.1,
PDCD1, CD37, KLRK1, ATP8A1, HOPX, GNG2, RALGAPA1, RP11-489E7.4,
SCML4, AOAH, CD247, ZNF331, SLC38A1, ITGA4, CARD11, EVI2B, FASLG,
RPLP1, IFNG, EOMES, CHST12, STK17B, DAPK2, LMNB1, TC2N, ID2, TANK,
RASAL3, THEMIS, PTGER4, CTLA4, CD48, STAT4, FNBP1, ICAM3, RAB27A,
TNFRSF1B, KIAA 1551, GFI1, PDCL3, PIK3IP1, HAVCR2, APOBEC3H, RCAN3,
EZR, TMEM2, AKAP5, SUPT3H, MIAT, WIPF1, LRMP, FAM159A, BIN2,
ARHGAP9, RP11-35612.4, AC002331.1, IDI1, ZBTB32, DUSP5, BCL11B, CAMK4,
SLC7A51, IRF4, DOK2, LAT, LIMD2, RP11-51J9.5, CASP8, SEP1, RP5-
1171110.5, VCAM1, RHOF, YPEL1, PBX4, PRKCH, IPCEF1, OXNAD1, PPP1R2,
ZAP70, SOCS1, P2RY10, BIN1, DUSP10, LYST, RHOH, IGHG3, TRAF3IP3,
AC006129.2, CXCR3, TOX, SLAMF6, PRKCB, FMNL1, LCP1, TOB1, IGKC, IDS,
SEMA4D, PABPC1, GALM, ZBTB1, LINC00239, ITGB7, TIGIT, CKLF, SMCHD1,
HPGD, IKZF1, DEF6, IL7R, INPP5D, GPR132, LPIN1, REL, ARID4B, RABGAP1L,
AC058791.1, MALT1, AHI1, IL10RA, TSPYL2, CELF2, EPB41, PDE4B, PRMT9,
GPSM3, ABI3, EVI2A, RSRP1, IGHG1, FYB, KMT2E, RNF166, AIM1, SC5D,
ARL4C, ANKRD12, IGHG4, SOX4, HBB, GCC2, FAM118A, PFKFB3, SYNE2,
UBE2S1, ZEB2, ARHGAP15, METRNL, AKAP9, PRDM1, IGLC2, SEP6,
FAM133B, AKAP13, HCLS1, FAM107B, PMAIP1, HSPH1, ANXA6, MYADM,
GBP2, DNAJA1, CDC42SE1, TGIF1, HSP90AA1, and PIM3.
Cluster 2: PLVAP, NPDC1, EMCN, SLC9A3R2, SRP14, VWA1, PECAM1,
CLEC14A, PODXL, RAMP3, ENG, ADGRL4, PRCP, HSPG2, PTPRB, ESAM,
SOX17, NOTCH4, SOX18, CYYR1, INSR, SLCO2A1, VWF, MMRN2, DUSP23,
LIMS2, FAM110D, CDH5, CALCRL, CD34, FAM167B, APLNR, TEK, CD93,
PALMD, AQP1, COL15A1, ADCY4, ICAM2, EGFL7, MCTP1, NOSTRIN, HEG1,
FLT1, HYAL2, EFNB2, NUAK1, ADAM15, FKBP1A, BMPR2, TCF4, SELE,
GRB10, SPRY1, CRIP2, SOX7, PTPRM, ADAMTS9, MEOX1, RAMP2, MECOM,
DLL4, PLPP1, JAM2, MCF2L, PCDH17, SHROOM4, S1PR1, EXOC3L2, LDB2,
CXorf36, TMEM88, KDR, PIK3R3, ARHGEF15, PREX2, ERG, RAPGEF4,
TMEM255B, RAPGEF5, MTUS1, DNASE1L3, SLC6A6, STC1, EPAS1, DOC2B,
IL6ST, ZNF366, NEDD9, NOVA2, TGFBR2, RASIP1, ICAM1, TSPAN7, FRY,
ROBO4, PCAT19, FGD5, ACVRL1, F2RL3, SPTBN1, CPLX1, PNP, HECW2,
ARAP3, CNTNAP3B, PLXND1, KCNN3, PTPRG, CLEC1A, APOLD1, GIMAP6,
SH3BGRL2, TSPAN18, SPRY4, C2CD4B, MMP15, ARHGAP29, SCARF1,
MYCT1, TMEM233, SPNS2, FNBP1L, SEMA3F, FAM171A1, ELK3, NES,
ADGRL2, BCAM, ID1, PDGFB, NFIB, CCDC3, HOXD8, PLEKHG1, RCAN1,
TM4SF1, ADGRF5, CNKSR3, IL3RA, DYSF, EPB41L4A, TIE1, CRIM1, TM4SF18,
LMO2, YPEL2, THBD, RPS6KA2, SMAD6, AFAP1L1, NEURL1B, ARL4A, ITIH5,
HHEX, CMTM8, KIAA1462, AIF1L, CXCL12, KLF2, C8orf4, GNAI2, TMEM204,
NRP1, PVRL2, KANK3, LRRC32, ETS2, TSPAN12, LMCD1, CLDN5, ITGA61,
LYL1, CDC42EP3, SLC2A3, GNG11, SEMA6B, DUSP6, PLAT, TNS2, A2M,
ADAMTS1, SH3BP5, RAB13, CADPS2, TRIOBP, TGM2, COL4A1, CD200,
PDLIM1, EDN1, LAMA4, EFNA11, MALL1, TSHZ2, GRASP, GIMAP7, COL4A2,
GIMAP4, CAV11, SOCS3, RHOJ, SPARCL1, SNCG, GATA2, PALD1, ITM2A,
DPYSL2, MCAM, ENTPD1, PTRF, ANGPT2, SDPR, GSN, C1orf54, DLC1,
ECSCR. 1, CD591, NR2F2, PDK4, APP, VIM, NID1, EMP11, PLPP3, CD9, HLA-E,
PTMA, TSPAN13, MEF2C, IGFBP7, ITGA51, RBPMS, CNN3, GJC1, KTN1,
JAG2, MATN2, TIMP3, IFI271, ZNF467, PTPN14, ABL2, RASA4, TMEM44,
PRKCH1, ALPK3, PPM1F, ZEB1, PRKCDBP, ACE, CDH13, SCN1B, LPAR6,
FRMD4B, VASH1, CFP, SMAD1, RHOB, TMCC3, RAI14, PTPRN2, EML1,
PTP4A3, MAST4, SOX13, RGS16, PKIG, EXOC3L1, AKR1C3, MSX1, SGK1,
JAM3, ID3, TSC22D1, SNAI1, EDNRB, TTC28, ADIRF, COL13A1, TSPAN15,
LIMCH1, ZFP36L1, RAI2, ITPKC, CDA, PEA15, ARHGEF12, BDKRB2,
ANKRD28, PIM31, ICAM4, FNIP2, SLC35E4, PTPRE, SERPINE1, RCAN2, MT-
ND1, SH2B3, YES1, MT-ND2, NEAT1, CSF2RB, LAMB1, OLFML2A, FXYD6,
TCF7L1, SVIP, STAB1, PROCR, PXDN, KBTBD2, CD79B, PHACTR4, KMT2E1,
TGFBR3, FNDC4, ADAMTS4, CHD7, PROS1, FAM101B, IGFBP3, PHACTR2,
CD81, TTC9, DAAM11, HES1, MGAT4A, CDKN1A, MAP4K4, AKAP131, UACA,
ATP11C, CTNNB1, LEF1, NFAT5, TMEM21, KIAA1147, UNC119B, PGF,
ZSCAN18, FAM43A, MIR222HG, LAMC1, KCTD12, FES, IFI44L, ABI31, SYNE21,
OSBPL10, APLP2, SEMA4C, GJA1, CPD, RP11-553L6.5, DDAH2, TXNIP,
IFITM3, LRRC8A1, EFNB11, DENND5A, UGCG1, MT-ND5, IGFBP5, CD1091,
ARHGAP27, RP11-108M9.4, GBP4, SRGAP2B, RSRP11, SLC26A2, ARRDC3,
SRGAP2C, ARID5A, ADAM10, NFATC1, NCOA71, NUMB, NFATC2, DGKH,
JUND, IRAK3, SLC38A21, DDIT4, TRIB11, VEZF1, CCL2, ZFP36, S100A101,
GPATCH2L, CCND1, NSRP1, JAG1, MDM4, KATNBL1, XAF1, IVNS1ABP,
HIF1A, RGCC1, NUCB2, OCIAD21, ESF1, IFITM11, BST2, and RP11-115D19.1.
Cluster 3:
PSAP, CTSB, LGMN, CTSS, MS4A4A, FCGR2A, PLD3, MS4A7, PLA2G7, CD68,
TREM2, CTSZ, CD163, FCGR3A, LIPA, C1QC, PLTP, LAIR1, FTL, CD14, CTSL,
GRN, FPR3, SLC11A1, C1QA, C5AR1, CTSD, C1QB, AQP9, FPR2, NPC2,
CREG1, TMEM176B, CAPG, LAPTM5, PILRA, APOC1, LILRB2, FCER1G, SDS,
ITGAX, SLC7A7, CCL3L3, CCL18, TYROBP, IGSF6, GPR34, CCL3, GLUL,
CLEC4E, FCGRT, MCEMP1, SLC16A10, CSF1R, SNX10, FBP1, SLCO2B1, IL1B,
MMP12, RNASE1, ASAH1, GPX1, LILRA6, CYBB, RNF130, ACP5, AIF1, HLA-
DMB, MS4A6A, MRC1, FAM26F, FOLR2, PLAUR, NCF2, MMP19, OLR1,
SIGLEC10, ITGAM, LILRB3, GPR84, C3AR1, SLC40A1, BCL2A1, CYBA, TNF,
ITGB2, LY96, MPP1, TMEM176A, LINC01272, VAMP8, HLA-DMA, ACSL1,
CSF3R, CD300A, MERTK, SDC3, EREG, SPP1, CLEC7A, CD300E, LYZ,
CLEC5A, TFRC, CECR1, SERPINA1, MPEG1, MIR3945HG, RBM47, CCL20, C2,
SPI1, TBXAS1, SLC43A2, ABCA1, FCN1, ALOX5, CCRL2, FGL2, FTH1,
RNASET2, SLC8A1, GPNMB, G0S2, LCP2, NLRP3, RAB20, MNDA, HCK, IL411,
KMO, TTYH3, VMO1, C15orf48, FPR1, APOE, FCGR2B, GCA, ADGRE2, PLEK,
CD86, MMP9, SAT1, IL10, RNASE6, HLA-DRB1, NAMPT, CCL4L2, CXCL3,
CXCL16, CXCL5, TREM1, CXCL8, TLR2, RASSF4, KCNMA1, NAIP, NFKBIA,
STAB11, DMXL2, ZFAND5, HLA-DRA, OSM, ALCAM, IFI30, C1orf162, C5AR2,
SOD2, PTAFR, CLEC4A, ANKRD22, NCF1, BASP1, PIK3AP1, P2RY13, HLA-
DRB5, LUCAT1, LST1, FGR, LYN, CTSH, GLIPR2, GK, HLA-DQA1, CD74,
ARRB2, TYMP, MAR1, MANBA, CXCL2, CXCL9, TCHH, FAM49A, KCNE3,
STAC3, STX11, TNFSF13B, GM2A1, HLA-DPA1, LY86, HLA-DPB1, CD4, ,
NIP21, KYNU, DNAAF1, ANPEP, HLA-DOA, HLA-DQB1, DAB2, LAT2,
RASGEF1B, NINJ1, RNF149, CCL41, EPB41L3, CSTB1, TLR4, IER3, IL181,
AOAH1, RNF144B, B2M, HMOX1, IL1RN1, FMN1, LRRK2, CD84, PLXNC1,
APOBEC3A, PHACTR1, RP11-76E17.3, AREG, GNA13, MAFB, IL1R2, MXD1,
SH3BGRL3, CD83, CPVL, HCLS11, LCP11, CD63, CSF2RA, GPR183, CLEC10A,
CPM, HCST1, PLIN2, ZEB21, TNFAIP2, CXCL101, FYB1, RGS2, SRGN, ALDH2,
OPN3, CD531, RGS10, CD481, TMSB4X, CSF2RB1, LITAF1, PKIB, S100A91,
GPSM31, EVI2B1, KCNJ2, GLRX, SLC16A3, ZG16B, VAMP5, FES1, EBI3,
TNFAIP3, IRAK31, SYNGR2, PNRC1, THAP2, VEGFA, TLR1, ADAM8,
PLEKHO1, HBEGF1, RASSF2, UCP2, SLC7A8, IFIT2, GNGT2, SDSL, CAMK1,
AKR1B1, ITGB71, TET2, GCHFR, NRIP3, EMP3, SORL1, DUSP21, MARCKS,
CXCL1, ANXA5, NFKBID, PTGS2, RP1-239B22.5, RHOU, CHI3L1, HVCN1,
CH25H, OTUD1, PPIF, LGALS3, INSIG1, DOK21, ISG151, CIITA, SERPINB9,
MIR155HG, SGK11, TNNI2, TLR5, CHMP1B, PRKCD, MAP3K8, CD44, REL1,
CD36, TNS3, HK2, QKI, KCNQ1, CXorf21, RP5-1171I10.51, PDE4B1, CFLAR,
LINC00936, NFKB1, ZNF3311, IRS2, DOCK4, MT-CO1, NEAT11, MYO1G, ARC,
SLC15A31, IL6, YWHAH, HLA-B, ABL21, TAGAP, HSD3B7, SH3BP51,
ADGRE51, TGFBI, GLA, HIF1A1, GAB2, CEBPB, CD811, CASS4, ARL4C1,
ST141, NABP1, ABI32, PFKFB31, CDC42EP2, S100A81, GBP5, FN1, HSPA6,
ARL5B, HLA-DQA2, NFIL3, TNFAIP6, GALM1, LPAR61, METRNL1, GPRIN3,
RP3-395M20.12, NFKBIZ, RP11-386I14.4, TNFAIP8, SLC2A31, PEA151, UBD,
RSAD2, CD40, TXNIP1, NBN, MT1G1, NUPR11, SCD1, IFI61, FNDC3B, THBS1,
FOSL2, APLP21, IFIT31, ADAM91, ZFYVE16, PPP1R15B, GADD45G, SATB1,
NRP2, MX2, TIMP1, PRMT91, IVNS1ABP1, GBP1, SIPA1L1, PDK41, SEPP1,
MT1F, NUMB1, GBP41, KLF6, WTAP, PPP1R15A, ADAM101, SDC2, IER5,
ZC3HAV1, HSPA1B, CRIP1, S100A102, DDAH21, HLA-A, PMAIP11, SLC25A37,
SELK, CD521, EIF4A3, SLC3A21, and OSBPL8.
Cluster 4:
HTRA1, GALNT1, NBL1, COPZ2, S100A13, CDH11, ASPN, MXRA8, COL16A1,
ANGPTL2, COL12A1, KDELR3, CLEC11A, P3H4, PLEKHA5, GAS1, MXRA5,
FKBP10, FMOD, ALPL, OLFML2B, CERCAM, PCOLCE, FBLN1, OLFML3, ANKH,
AEBP1, BGN, LAPTM4A, THBS2, RCN3, BICC1, ENAH, CTGF, COL1A2, CTSK,
SPARC, COL1A1, EFEMP2, SSPN, PRRX1, PFN2, PRSS23, LPAR1, MMP2,
COL3A1, COL5A1, MFAP2, SNAI2, BAMBI, HAPLN1, FAP, COL5A2, PODNL1,
COL6A3, ANTXR1, SFRP2, COL8A1, LUM, EDIL3, DLX5, SCRG1, VCAN,
RAB31, FGFR1, RGS3, SERPINF1, EMILIN1, ZFHX4, IGFBP4, SERPINH1, MDK,
PALLD, CTHRC1, SMOC2, CLMP, NNMT, GJA11, POSTN, DCN, CYR61, CPE,
LOXL1, SDC21, COL6A1, ERRFI1, GLT8D2, PDGFD, RARRES2, MMP14,
COL6A2, GOLM1, SEPP11, ADAMTS2, FBN1, SULF1, MARCKS1, LRP1, VASN,
GAS6, FN11, HTRA3, FOS, SERPINE2, TWIST1, CCDC80, ENPP2, PMP22,
LTBP2, RHOBTB3, CYBRD1, FBLN2, TIMP2, TNC, IGFBP51, CLU, EGFL6,
NREP, MFGE8, FXYD1, NUDT4, ISLR, CNN31, AXL, C1S, VIM1, COX7A1,
TAGLN, THBS11, THY1, MGP, LGALS1, FSTL1, SERPING1, DKK3, C1R,
HSPB2, MMP11, ID31, CDC42EP31, CST3, TGFB1I1, S100A4, GADD45B, NFIX,
ID4, PDGFC, CRABP2, IGF1, CTNNB11, FAM46A, IGF2, HES11, JUN, MT1E1,
TPST1, SEMA3B, FAM134B, CXCL121, ZFHX3, SRPX, ARL6IP5, STEAP1,
SERTAD4, FXYD61, SLC44A3, KCNQ1OT1, CFB, VCAM11, COL14A1, TIMP31,
APP1, EGR1, RERG, PTPRS, MT-ND51, LURAP1L, FLRT2, LIMCH11, CADM1,
MIR99AHG, TGFBI1, TSC22D11, GOLIM4, MAFB1, NEXN, FBXO32, UNC5B,
GPM6B, GSTM3, PTGES, , SH3PXD2A, EFNA5, CYGB, GGT5, FNDC41, EBF1,
DIO2, C10orf10, LOXL2, MT-ATP6, RASD1, MT-ND4, CD276, IRX31, RAI141,
CKAP4, ZEB11, FARP1, PTK7, C1QTNF1, CDKN1C, TMEM158, SEMA3C,
NRP21, PRRX2, MAP1LC3A, MT-CYB, KCTD121, FBLN7, FAM20C, PMEPA1,
CSF1, LAMB11, NFATC21, MT1M1, CDKN2A, GEM, , SOCS31, MAP4K41,
PDPN, GADD45G1, RIN2, ZMAT3, MYADM1, NFKBIZ1, APOL21, JUNB,
ZFP36L11, ESF11, CKB1, INTS6, IRF1, B4GALT1, PDIA3, NSRP11, CXCL141,
MT2A1, MT1X1, DDIT41, VMP11, CFD, and ZFP361.
Cluster 5:
BATF, ICOS, ICA1, RTKN2, CCR6, AC145110.1, TNFRSF4, SARAF1, IL32,
LAIR2, FOXP3, TNFRSF18, CARD16, LINC01281, AC017002.1, IL2RA,
AC133644.2, CD27, LTB, SPOCK21, ADTRP, METTL8, CD177, PBXIP1, TIGIT1,
RP11-1399P15.1, CD21, F5, TRAC1, SELL, ZC3H12D, KLRB1, HS3ST3B1,
DUSP16, SKAP1, IPCEF11, MAL, TBC1D4, CD28, TNFRSF9, CTLA41, RHOH1,
SLAMF1, MFHAS1, PIK3IP11, LINC002391, RP11-553L6.2, IL7R1, TRBC21,
GMFG, RP11-347P5.11, ACP6, LEPROTL11, RP11-51J9.51, STK41, PIM2,
LAX1, CD2471, TRBC11, PPP1R21, PASK, LAT1, PBX41, SIT1, CCR7, ICAM31,
CYTIP1, TESPA1, NINJ2, SATB11, FCMR, FAM107B1, IL18R1, SMAP21,
ZNF831, CREM1, RPS271, ITK, SEP11, CD71, ARHGDIB1, CD3E1, TSC22D31,
TXK, CLEC2D1, SRGN1, IL2RG1, BTG11, SAMSN11, P2RX5, MYB, FYN1,
CXCR31, SLA1, STAT41, LCK1, RP11-712B9.2, CTD-2020K17.4, CD3D1,
GPRIN31, DUSP41, RGS11, OXNAD11, RBM381, RPS292, CD3G1, PTPRC1,
RASGRP2, FRAT1, CXCR41, FAM174B, SEMA4D1, CD532, SYTL31, CORO1A1,
PCNX, CD961, LTA, BCL2, TTN, IL10RA1, STK17B1, CELF21, GATA3, RIC3,
TAGAP1, CNOT6L1, PYHIN11, PKIA, CASS41, LEF11, BIRC3, SUSD3,
AC002306.1, P2RY101, RP11-138A9.21, SLAMF61, CD226, ATP8A11, RCAN31,
SLC27A2, INPP5D1, UCP21, RORA, SYTL1, SGPP2, SC5D1, IKZF11, IKZF2,
LINC01550, FAM129A1, CAMK41, RP11-35612.41, AP003774.1, SPNS3, RCSD1,
IGFL2, RP11-25K19.1, GK-AS1, GPR1711, EVI2A1, FAS, ZNF165, GPR1321,
CEP19, LINC00426, RPLP11, SP140, DENND1C, SH2D1A1, DEF61, MIAT1,
ZNF101, UGP2, WNT10A, ACAP11, S1PR41, TRAF3IP31, MALT11, PLIN21,
TRAF1, ZAP701, HBA1, ISG201, ETS11, SEP61, LINC00891, CDC42SE21, NMB,
CXCR61, ITM2A1, TNFRSF25, SH2D2A1, AC058791.11, CCR4, CASP81, IL21R,
CTD-3222D19.8, RP11-796E2.4, APOBEC3H1, GK1, SLC1A4, IKZF3, OSBPL7,
CD61, AIM11, RHOF1, FAM184A, FAAH2, CCR5, ARHGAP11B, PMAIP12,
RAB33A, MAGEH1, AGAP2, ZNF92, PABPC11, EMB1, RAB39B, BTLA, LPIN11,
FBLN71, LIMD21, RP11-138A9.11, LINC00649, IL18RAP, FANK1, GPCPD1,
ELF11, CD841, RGCC2, HBA2, AKNA1, JAKMIP1, NR3C1, TSPYL21, RAC21,
EVI2B2, RNF19A1, COL9A2, ARHGAP91, RP11-166P13.4, CHST7, TMEM2381,
CORO1B, HLA-A1, ZBED2, LY9, AC016831.71, CDC14A, POLM, BEX2, DOK22,
HPGD1, ARHGAP151, TRIM4, ANKS1B, ATP6V0E2, RPL34, UBR1, RP5-
827C21.4, TRIM59, CD70, LINC00861, TPT1, FYB2, MTF1, CDKN1B, PDCD41,
GZMM1, MAP4K1, MORN3, PDE4B2, TNFRSF1B1, TTC39C, GALM2, ZFP36L21,
MFSD8, ARAP21, IL12RB1, TNFSF14, PIGA, MIR3142HG, GIMAP71,
AC006129.21, CTD-2020K17.1, EZR1, GTSF1, ANKRD44, IGHV3-23, EPB42,
CD5, TBX21, PTPN71, GFI11, PDE3B1, GRAP, GNG21, SNIP1, P2RY8, TOX1,
PPP2R5C1, APBB1IP, MCL11, SIRPG, EPB411, RP5-1028K7.2, IL2RB,
RAB11FIP4, ARSK, CD41, FAM134B1, CYSLTR1, PGAP1, LMOD3, RASSF5,
TCAF2, TSPAN32, ZNF3312, BIN21, SOCS11, KIAA0319L, EVL1, TANK1,
ZNF17, TMEM156, S100A41, ZDBF2, FMNL11, RASGRP1, FAM46C1, ZBTB321,
FXYD2, SMCHD11, EBI31, GLCCI1, PPM1K, AKAP51, TMEM154, IDI11, ANO9,
PARP15, FAM159A1, HMHA1, RAB11FIP1, RASAL31, RNF1251, PPP1R16B,
RNGTT, RP11-489E7.41, NUCB21, CNNM4, PCBD2, GCC21, TSTD1, FGD3,
RBM34, TBC1D10C1, RP5-908M14.9, AC104820.2, TNFAIP81, TACC3, LBH,
ANKRD121, NDC80, MYO1G1, CBLB1, RBPJ1, C1orf228, CCNG2, GCNT1,
TSGA10, C12orf60, AC002331.11, CCNH1, GYPC, RP11-660L16.2, CEP85L,
ASB21, CENPM, UBASH3A, RABGAP1L1, BCL11B1, GBP51, ZBTB11, TMPO,
SPN, ZNF292, ARID4A, PDCD11, FAM118A1, CD691, SELK1, IL27RA,
PTPN221, GBP21, NUFIP1, GPR155, CCDC159, NXPE3, ARID5A1, ADAM19,
LINC01588, IL16, SCML41, AP5Z1, YPEL11, DDX20, SLC5A3, CHAMP1, RGL4,
FKBP5, ZNF37A, SLC38A11, SNAI3, H2AFZ1, ARID4B1, PRKCH2, PTGER41,
CCDC88C, GPSM32, TRIM11, MAF, THEMIS1, PDCL31, BIN11, ARL6IP51,
HERC5, ARID5B, TGIF11, TRAT11, DDHD1, SYNE22, SESN3, PDE4D1,
RPS6KA5, PFKFB32, IDS1, PRDM11, MTMR6, NABP11, IRS21, CFLAR1, PFN1,
DUSP101, LAG31, GLRX1, WIPF11, CCND2, TOB11, MIR4435-2HG, LYST1,
VPS37B1, CITED2, KDM5A, FRMD4B1, REL2, COX20, APOBEC3C, TSHZ21,
PIK3R11, PKM1, and HSP90AA11.
Cluster 6:
PLAC9, NR4A1, CFH, LTBP4, CRLF1, GPC3, LEPR, CES1, FOSB, C3, C1R1,
MEG3, CFD1, TWIST2, AKAP12, DIO21, FHL1, C1S1, CYBRD11, SQSTM1,
SERPING11, CCDC801, FSTL11, KLF4, MGP1, CLU1, C11orf96, SERPINE21,
CEBPD, PDLIM3, ATF3, EGR11, TNFAIP61, FBLN21, SOD3, LMNA,
SERPINF11, DCN1, CEBPB1, TIMP11, S100A6, RARRES21, NNMT1, MMP21,
LUM1, SELM, FBLN11, IGFBP41, CD631, CREB5, CST31, FBLN5, SFRP21,
GSN1, BTG2, TIMP21, MEDAG, FXYD11, CYR611, ISLR1, FILIP1L, EFEMP1,
LRP11, JUND1, APCDD1, IGFBP52, FGFR11, MAP1B, SRPX1, OSR2, NTM,
GPRC5A1, CPE1, FST1, STEAP11, NR4A2, JUNB1, GADD45B1, VGLL3,
LTBP21, WNT5A, CTHRC11, FBN11, CTGF1, TPPP3, DUSP1, NTRK2, IGFBP61,
GEM1, A2M1, STEAP2, HSPB8, IL61, COL8A11, C8orf41, SOD21, FABP3,
GREM1, CD812, PBX1, MFAP5, PAPPA, CRABP21, TGFBR31, RRAD1, LAMA2,
PLPP31, RASD11, EGR3, PHLDB1, MT1X2, AKR1C1, ID32, OLFML31, IGF11,
CRYAB, GPX3, CXCL13, TNFRSF12A1, DNAJB1, KDM6B, PDGFRB, PRRX21,
PTGS21, COX7A11, CCL21, EGR2, CRISPLD2, RHOB1, HAPLN11, SERTAD1,
ADM1, COL14A11, DKK31, HTRA31, NTN4, MME, PHLDA1, SPON2, DPYSL21,
IER31, IER2, FOS1, PIM1, MT2A2, NFIX1, RHOBTB31, RAI21, MGST11,
SEMA3B1, NFATC11, GLT8D21, CDKN1A1, NR4A3, VASN1, ARID5B1, RND31,
ANTXR11, CXCL21, WTAP1, MAFF, SFRP1, EIF4A31, AXL1, ECM11, H3F3B1,
PROS11, PNRC11, TSC22D12, RERG1, PMP221, TNFAIP21, EDIL31, MEST,
PTX3, BICC11, MEIS2, MYC, MYADM2, GLIS3, NFIL31, PMEPA11, IGFBP2,
GSTM31, DNAJA11, TUBA1A, APLP22, JUN1, PPP1R15A1, TWIST11, SSPN1,
TRIB12, DDAH22, CCDC71L, NFKBIZ2, FAM46A1, ZFP36L12, PTGES1,
NFATC22, SEMA3C1, TIMP32, BHLHE40, RDH10, CTD-3252C9.4, CSRP2, F2R,
NCOA72, LAMA41, CDKN1C1, PTPRS1, PXDN1, GADD45A1, FAM20C1,
LIMCH12, PHLDA21, HMOX11, DST1, ANXA51, HSPA1A, ARL6IP52, THBS12,
PTK71, PDPN1, GLA1, HSPA1B1, RAB311, CHMP1B1, FAP1, DAB21, NFIB1,
GAS61, TNFSF13B1, TUBB2A, WIPI1, KLF21, ZFP362, EMP31, MT1M2,
CELF22, NAF1, FAM133B1, TGIF12, TNC1, CD55, PHACTR21, CCND21,
SLC25A371, ABL22, LMCD11, IRF11, KCNQ1OT11, RGS161, OSBPL81,
PEA152, FAM215B, MAP1LC3A1, INTS61, ZNF3313, GYPC1, NUCB22, UGP21,
EIF4E, CRIP11, and TNFAIP31.
Cluster 7:
COX4I2, CDH6, DSTN, CCDC102B, RGS5, SEP4, PPP1R14A, COL18A1,
KCNJ8, EPS8, NDUFA4L2, EFHD1, WFDC1, OAZ2, ISYNA1, NOTCH3, LYPD1,
PLXDC1, LHFP, MAP3K7CL, GUCY1B3, MYLK, ITGA1, MYL9, EGFLAM, ABCC9,
STEAP4, GJC11, PARM1, ACTA2, TINAGL1, FAM13C, SYNPO2, THY11, TBX2,
CALD1, ID41, TGFB1I11, PTP4A31, KCNE4, PDGFRB1, MFGE81, C1QTNF11,
CRISPLD21, RERG2, ENPEP, EGFL61, MAP1B1, IGFBP71, TPM2, SOD31,
TAGLN1, CYGB1, GJA4, GUCY1A3, MCAM1, TPM1, EBF11, SPARCL11,
FILIP1L1, ADIRF1, CPM1, ITGB1, COL4A21, NR2F21, PHLDA11, PGF1,
COL4A11, TPPP31, ID33, C11orf961, ADGRF51, COL6A21, MGP2, ADAMTS41,
CPE2, SMOC21, PTMA1, IGFBP21, CHN1, RCAN21, RBPMS1, SERPINI1,
COL14A12, SPARC1, CNN32, GUCY1A2, OLFML2A1, EPAS11, CEBPD1, AXL2,
NDRG2, ARHGAP152, TFPI, HEY2, GEM2, CSRP21, PDLIM31, PRRX11,
COX7A12, PPP1R12B, HES4, SERPING12, PEAR1, ANGPT21, HSPB21,
GPX31, LGALS3BP, COL5A3, MOCS1, MAP2, SEMA5A, RASD12, NFASC,
PDGFA, LURAP1L1, SELM1, LINC00152, EDNRB1, NEXN1, GGT51, CSPG4,
VASN2, LINC00839, SOX131, PTGIR, PRKCDBP1, SCIN, CD248, EDNRA,
MEF2C1, ADAMTS11, OLFML2B1, LPP, GPM6B1, LAMC11, RAMP1, GPR4,
UACA1, PALLD1, TIMP12, CYP26B1, RASGRP21, FXYD12, LAMA42, TEX41,
NUDT41, NREP1, SYDE1, HES5, RGS4, TNS21, SYTL2, JAG11, NID11, HOPX1,
SFTA1P, BATF3, FBXO321, DLC11, IFITM2, C2CD2, SDC22, LOXL21, PHYHD1,
ADAM12, APOLD11, NES1, TRNP1, PMEPA12, BCAM1, CD361, SOCS32,
DLX51, TNFRSF21, SEPHS2, NEURL1B1, MIR4435-2HG1, GSTM32, MEST1,
PKIG1, CFH1, ADGRL21, IFITM31, CHCHD10, SNAI21, FOS2, FARP11,
TMEM2041, ARMCX2, ARID5B2, CKM, ANXA61, DKK32, FJX1, ARHGAP24,
TSPAN121, PARD3B, FHL11, CREB3L2, LRRC321, CDKL5, PTRF1, TUBB2B,
LBH1, JUNB2, TLE1, PROCR1, JUN2, ASAP2, RP3-325F22.5, TNS31, PBLD,
PPP1R15A2, C10orf101, RP3-395M20.121, SDSL1, SMIM10, CBFA2T3, ZFHX31,
USP13, CC2D2A, SHF, PDK42, SPON21, KRT181, MYC1, LGALS11,
MAP1LC3A2, PROS12, ARHGEF121, PHLDB11, HLX, LINC00926, GBP22,
CDKN1A2, RP11-553L6.51, RHOBTB32, CACNB1, FAM127C, DNAJC27,
RCSD11, BST21, ZFP36L13, PROSER3, GADD45B2, FAM46A2, ITGAV,
CRIP12, MT1M3, SEMA4C1, HES12, CEBPB2, LAMB12, ZEB22, SRGAP1,
SENP3, NRP11, PAG1, EGR12, ATF31, HIF1A2, MDK1, TUBA1A1, H1F0,
CXXC5, IFIT32, THOC2, CDH131, ANTXR2, SEC23A, CHST71, SERTAD11,
RPS6KA51, MAFF1, INTS62, CTA-29F11.1, IRF12, ZMAT31, CCND11, GLA2,
CCL22, MX21, FAM213A1, RHOB2, FSTL31, NSRP12, ZFP363, KCNQ1OT12,
DDIT3, CDC42SE11, ATP2A21, TUBA1B, ISG152, COX201, CTD-3252C9.41,
IFI62, FAM133B2, and HLA-F.
Cluster 8:
SSR4, XBP1, FKBP11, MZB1, IGHA2, DERL3, JCHAIN, IGHA1, IGHG41,
IGHG11, IGHG31, IGKC1, IGHG2, CD79A, IGLC3, IGLL5, FCRL5, IGLC21,
HERPUD1, ITM2C, PIM21, EIF4E1, KLF61, NUCB23, CDKN1A3, ANKRD281,
MALAT11, HSPA51, CITED21, DUSP51, FAM107B2, BTG21, and HSP90B11.
Cluster 9:
LGALS12, SPON22, SELM2, LOXL11, COL6A11, CTHRC12, TPM21, COL6A22,
FN12, COL6A31, COL5A11, COL5A21, COL1A11, COL3A11, CALD11,
COL1A21, MYL91, AEBP11, LUM2, SPARC2, THBS21, BGN1, DCN2, PCOLCE1,
MEG31, EMILIN11, RCN31, RARRES22, VCAN1, ADAMTS21, SULF11, HTRA32,
FSTL12, ISLR2, FBLN22, MMP22, SFRP22, NTM1, CTSK1, COL8A12,
CERCAM1, EFEMP21, POSTN1, TAGLN2, C1R2, TWIST21, SERPINH11,
TPM11, CDH111, C1S2, PRRX12, TIMP13, CRISPLD22, COL7A1, FKBP101,
STEAP12, PDGFRB2, GREM11, THY12, FBN12, CTGF2, WNT5A1, MFAP21,
C11orf962, TIMP22, SCG5, TNFAIP62, MXRA51, RGS31, CLMP1, LTBP22,
ANGPTL21, BICC12, FAP2, KDELR31, IL11, NREP2, OLFML32, COL5A31,
TNC2, TMEM45A1, COL16A11, GGT52, TWIST12, CRABP22, TGFBI2, MMP141,
COL12A11, SERPING13, GAS11, FGFR2, SUGCT, PAPPA1, ENAH1, FGFR12,
NNMT2, LOXL22, TNFRSF12A2, PRRX22, ASPN1, CYR612, GLIS31, CHN11,
STEAP21, MMP111, FBLN51, PODNL11, PTK72, MFAP51, MME1, ANTXR12,
RHOBTB33, PI15, IGFBP42, IL24, CD2481, S100A61, PALLD2, CLEC11A1,
HSPB22, INHBA1, GPM6B2, ADAM121, LGALS3BP1, EGFL62, ID34, THBS13,
WNT5B, TGFB1I12, TMEM1581, GUCY1A31, NEXN2, AXL3, CCL23, ACTA21,
LAPTM4A1, DIO22, LPAR11, LAMA43, SFTA1P1, PMP222, PTGES2, CRLF11,
MMP1, EVA1A, IGFBP53, CRYAB1, RAB312, COX7A13, CYGB2, PXDN2,
PDGFC1, PRKCDBP2, SMIM101, CST32, MDK2, SSPN2, PDLIM32, PMEPA13,
CKAP41, TUBA1A2, SEMA3C2, GOLM11, DKK33, PARD3B1, VGLL31, CD2761,
FGF1, LAMC12, IFITM32, GAS62, IL62, NOX4, MEST2, CCBE1, PLEKHA51,
FAM19A5, GLIS2, MAPK8IP1, PHLDB12, MEDAG1, LINC001521, WDR86,
SNAI22, TPST11, CFB1, S100A42, PRSS231, FILIP1L2, PBX11, ANXA52, F2R1,
SRRM3, FBLN72, SCX, CREB51, DIXDC1, EML11, ERRFI11, SH3PXD2A1,
HES41, SEMA5A1, IGF21, LY6E1, TSPAN11, FAM20C2, CYP26B11, PDZRN3,
ARHGEF40, FAM20A, FAM167A, SPATA17, WIPI11, NDRG4, RND32, FARP12,
LAMA21, BPGM, DCBLD2, ITGAV1, FBXO322, PDLIM41, MRAS, BST22,
TIMP33, PHLDA12, ZFHX32, IFI63, BATF31, APCDD11, ITGB11, BACE1,
BAMBI1, NOV, ARMCX21, GPSM1, MAP4K42, TSC22D13, STC2, SERPINE11,
ANXA62, C2CD21, ZCCHC24, RASD13, FOXD1, PLAU1, ANPEP1, BMP2,
FAM63B, PSMG3-AS1, ITGA11, H1F01, PHLDA31, CEBPB3, RP11-553L6.52,
ITGA52, IGFBP31, MEIS21, HES13, SEC23A1, ISG153, RBPMS2, RGPD5,
HILPDA, EGR31, KCNQ1OT13, PLXNC11, DUSP11, GBP11, MT1E2,
CCDC71L1, ADAM191, RP3-325F22.51, WDFY1, ANGPTL41, CHPF2, PIM11,
ZBTB41, ATF32, PBLD1, CXCL15, HIF1A3, ARID5B3, DCBLD11, CEP57L1,
IFIT33, XAF11, B4GALT11, IFITM12, APOL22, DHX32, IFI44L1, ANTXR21,
IGFBP22, TNFRSF211, IFIT1, CREB3L21, FSTL32, RIN21, HSPA1A1, BNIP31,
MX22, SLC25A372, MAFB2, CTD-3252C9.42, ZFAND2A, AKR1B11, RRAD2,
HSPA1B2, ZMPSTE24, ADM2, CHMP1B2, BHLHE401, METRNL2, IRF13, and
HSPH11.
Cluster 10:
HMGB2, STMN1, HMGN2, HIST1H4C, CORO1A2, PFN11, HMGB11, GZMA1,
GNLY1, GZMB1, TUBB, NKG71, CD3D2, TYMS, CD72, TRAC2, CCL51,
ALOX5AP1, ACTB, TMSB4X1, RAC22, CTSW1, KLRD11, KIAA01011, TUBA1B1,
COTL1, CD8A1, AC092580.41, NUSAP11, CD522, TOP2A, LSP1, PRF11,
LAG32, DUSP42, CD8B1, CD3E2, CKLF1, LCK2, TRBC12, TRBC22, GAPDH1,
LDLRAD41, SH3BGRL31, SIRPG1, RAB27A1, TRGC21, CLEC2D2, CD3G2,
ARHGDIB2, AC002331.12, HAVCR21, CD22, APOBEC3G1, IL321, CENPF,
RRM2, ASPM, TIGIT2, HCST2, ISG202, TRAF3IP32, MKI67, IL2RG2, H2AFZ2,
GZMH1, CD962, UBE2C1, EVL2, RUNX31, PTTG11, BIRC51, CTLA42, GNG22,
GMFG1, IL2RB1, PDCD12, SAMSN12, AC133644.21, PTPN72, CDK11, CENPA,
CKS1B1, CDT11, ZWINT1, CENPE, IFNG1, MAD2L11, CENPM1, TPX21,
AURKB, CCNA2, PLK1, HIST1H1B, BATF1, SPC25, SEP12, RGS12, MAP4K11,
SLA21, GIPR, OXNAD12, NUF2, ARL6IP11, LCP12, HPGD2, CD2472, CYTIP2,
TBC1D10C2, SH2D2A2, FASLG1, KIF20B, HIST1H1D, ARHGAP30, CDKN31,
ATP6V0E21, SIT11, CASC5, RBPJ2, CD701, RBM382, SLC27A21, ACAP12,
SPOCK22, LRMP1, PIF1, KIF18A, TNFRSF91, ITGA41, TNFRSF181, MYO1G2,
TK11, SYTL32, RASAL32, PTPN222, CCR51, SKAP11, KIF22, EMB2, IL18RAP1,
TMPO1, LAT3, S1PR42, FMNL12, RPLP12, SH2D1A2, AURKA, ZBTB322, IRF41,
SPNS31, TNIP3, TOX2, SAMD31, CYFIP2, MYBL2, CHST121, SUPT3H1,
APOBEC3H2, HIST1H2BH, SPN1, CXCR62, P2RX51, SP1401, SLA3, KLRK11,
BIN12, CAMK42, APOBEC3C1, HMMR, CENPK1, NLRC3, CCL42, ATP8B4,
DLGAP5, THEMIS2, DOK23, GATA31, TANK2, IL21R1, CKS21, ENTPD11,
SPDL1, GPSM33, GSG2, CLSPN, LMNB11, ARHGAP11A, HSH2D, JAKMIP11,
CCNB21, PDE4D2, PCNA1, IL18R11, GRAP2, PBK1, GALM3, KLRC41,
UNC13D, TACC31, DUSP52, HMHA11, LIMD22, ID21, GTSF11, IKZF31,
NDC801, TESPA11, CDCA5, CD533, TNFSF4, DAPK21, NELL2, SUSD31,
CARD111, ZAP702, FGD31, PDCL32, TSTD11, RHOF2, ENO11, PDE3B2,
GFI12, FYN2, ITM2A2, ATAD5, SERPINB91, UCP22, TRAT12, HCLS12, PRR11,
NPW, AKAP52, FAM159A2, CD62, PPP2R2B, CLEC2B1, KIAA15511, ANXA63,
HLA-C, HOPX2, CD51, ITGB72, RP5-1028K7.21, BIN22, CBLB2, CD2261,
LYST2, RAB39B1, NUP210, GLCCI11, ADAM192, RP11-245D16.4, SEP62,
APBB1IP1, CDC201, TMPO-AS1, PYHIN12, RALY-AS1, ITGAL, SLC38A12,
MFSD81, TRG-AS1, ARHGAP11B1, RAB33A1, CD271, CASS42, AP003774.11,
SAMD10, SNAI31, RGL41, LIG1, ZNF383, DUSP102, FNBP11, LAYN, ELF12,
AGAP21, STK17B2, FAM83D, ADGRE52, SYTL21, RHOH2, DEF62, PMAIP13,
HIST1H1C, RASSF51, ZFP36L22, NINJ21, CDKN2A1, BTLA1, LINC004261,
PIK3IP12, HERC51, SLFN12L, ANKRD441, AHI11, HMGA11, JAK3, TNFRSF251,
GZMK1, CCNB11, IL12RB11, RP5-1171I10.52, PRDM12, ODC1, TBX211,
NDFIP21, C16orf54, B9D2, LINC015881, LPIN12, SLC1A41, MT1F1, GPR1551,
MX11, RCSD12, TSC22D32, IKZF12, SOX41, NFYB, HSP90AA12, TMEM2382,
VPS37B2, ANKRD282, AREG1, RNF1661, FAM118A2, COX202, ITM2C1,
PDLIM42, SLC5A31, RAB11FIP11, CORO1B1, MDM41, CFLAR2, CD821,
CEBPB4, DNAJB11, YWHAH1, and OAS11.
Cluster 11:
PKIB1, ACTB1, CLEC10A1, CPVL1, CSF2RA1, COTL11, S100B, HLA-DQB11,
TMSB4X2, HLA-DPB11, HLA-DPA11, LST11, HLA-DQA11, HLA-DRB51,
C1orf1621, CD741, HLA-DRA1, HLA-DRB11, AIF11, GPR1831, TYROBP1,
FGR1, C15orf481, INSIG11, RNASE61, SERPINA11, SPI11, LY861, LYZ1,
FCN11, PLEK1, IFI301, GOS21, CLEC7A1, HLA-DMB1, NLRP31, FAM26F1,
TSNARE1, CST33, HLA-DQA21, GPR157, PLD4, ANKRD221, MNDA1, BCL2A11,
HCK1, FCER1G1, BASP11, CD831, MAR11, FGL21, FPR11, CLEC4A1, GPX11,
LSP11, EBI32, CXCL161, CYBB1, HLA-DMA1, HCLS13, TMSB101, MMP25,
IGSF61, PLAC8, ITGB21, LRRK21, IRF8, TNNI21, CXCL91, FUT7, CD861,
LCP13, MS4A6A1, STX111, SLC7A11, ACSL11, PLAUR1, SAT11, C1QB1,
CCR71, CTSH1, CSF3R1, PPA1, CLEC5A1, PFN12, FCGR2B1, CFP1, GPAT3,
RASSF52, SNX101, SIGLEC101, ALDH21, GNA131, AREG2, SLC16A101,
SLC8A11, NR4A31, CCL221, SDS1, LYN1, REL3, RNASET21, RNF144B1,
CFAP45, OLR11, SRGN2, RASSF41, C1orf541, CYBA1, FBP11, NDRG21,
FAM49A1, MXD11, SERPINB92, VMO11, POU2F2, HDAC9, ITGB73, CLEC4E1,
IFNLR1, RGS101, PPIF1, PHACTR11, LAT21, SEP63, MARCKSL1, SUSD32,
PTPRE1, ALCAM1, P2RY131, KYNU1, HAVCR22, APOBEC3A1, ALOX51, GK2,
CLNK, LINC012721, EVI2B3, GPR841, IL4I11, UCP23, RP11-299J3.8, SDSL2,
RAB201, GRASP1, EREG1, ID22, CKLF2, PTGIR1, CBFA2T31, SYNGR21,
GPSM34, AOAH2, TNFSF13B2, HLA-DOA1, TREML1, JAML1, IL1R21,
DNAAF11, OSM1, PLEKHO11, GCA1, BATF32, TET21, CD534, TRAF11, EME2,
YWHAH2, MIR3945HG1, FRAT11, HLA-DQB2, PADI2, PLB1, GLIPR21, RP11-
76E17.31, SERPINB11, AC074289.1, CSF1R1, C17orf96, CD300A1, GK-AS11,
HMGA12, ASB22, HLX1, PLXNC12, SLC7A71, LAMP3, ZNF3661, RP5-
1171I10.53, NCF21, ADAM81, RAMP11, ANPEP2, METRNL3, C3AR11, DAPP1,
ADGRE21, CXorf211, SLC4A3, SLC43A21, LILRB31, AXL4, IL182, TYMP1,
SH3BGRL32, RGS21, EPB41L31, NFKB11, STK42, PNRC12, ARID3A,
MAP3K81, LITAF2, CST71, CIITA1, SLC35E41, BIRC31, RAB7B, CD300E1,
UBD1, PRR111, ST20, QPCT, NAMPT1, MYO1G3, IRF42, LUCAT11, DUSP53,
CFLAR3, IL1B1, TXN1, NUBPL, TLR21, PEA153, CH25H1, PALD11, XYLB,
NABP12, TNNT2, DNMBP, CHMP1B3, RP11-115D19.11, THAP21, PARM11,
ARHGAP22, CXCL81, VEGFA1, TCHH1, OPN31, NFKBIA1, DUOX1, TNFAIP82,
RASGEF1B1, HLA-B1, BCL11A, MIR155HG1, ZFAND51, ENO3, TNFAIP22,
ABI33, OTUD11, SAMSN13, C5orf34, SLC16A31, THEM4, SRGAP2C1,
ZFYVE161, ATP1B31, SUMO4, MALT12, LINC015882, LINC009361, CD401,
CAMK11, ADAM193, ICAM32, CEP191, GLA3, ARL5B1, ANXA64, CXCL102,
CCL201, EMP32, RP11-386114.41, CDC42SE12, TK12, VAMP51, IER32,
S100A43, APBB1IP2, PRKCD1, HBEGF2, CRIP13, GLUL1, TUBA1B2, NET11,
TAGAP2, C1orf561, ZC3HAV11, TXNIP2, FKBP51, HMGN21, TOB12, S100A103,
RNF213, GBP12, and MARCKS2.
Cluster 12:
CD79A1, BANK1, CD371, RP11-575L7.8, RPS272, COCH, HLA-DRA2,
ARHGAP241, RPS293, CCR72, RASSF6, RPLP13, TMEM1561, CD742,
RALGPS2, RP1-313I6.12, TMPO-AS11, CXorf212, STAG3, GORAB, CBFA2T32,
CD551, COL9A21, CD523, ANKRD442, TLR11, IGLC22, HIST1H1D1, CD832,
HOPX3, TSGA101, IL23A, EVI2B4, LY91, LTB1, ARHGAP301, IRF81, IGHG12,
GNG7, RAB11FIP41, TBC1D10C3, RP11-796E2.41, BCL21, TLE11, ANO91,
PARP151, IGKC2, RBM341, EZR2, AMN, MYO1G4, CCDC189, IGLC31,
CXCR42, BTG12, PRKCD2, CD692, CD79B1, LAT22, LYL11, ULK3, FCMR1,
ZNF274, SIPA1L3, LY862, AC006129.22, CENPL, HHEX1, CIITA2, SC5D2,
HVCN11, LINC009261, FAM159A3, CSRP2BP, BTLA2, INPP5D2, HERPUD11,
FCRL51, POU2AF1, PTK2B, MTMR61, FAM212B, MEF2C2, NAF11, ZNF1011,
NEK8, CCR61, TTN1, FAM43A1, PLD41, RNGTT1, RPL341, IL161, CAMKK1,
CD241, LIPT1, POU2F21, C12orf601, XXbac-BPG299F13.17, MT-ND3, LTB4R1,
SELL1, LCORL, GRAP1, SMAP22, ZNF921, GAPDH2, CD535, DAPP11, MAR12,
RASSF53, YPEL51, TMSB102, PCNXL4, C16orf541, LYN2, RIC31, LIMD23,
STRIP2, RASGRP22, CD482, AC137932.4, MACC1, FAM65B, TMEM1541,
NR4A21, TACC32, CPNE5, TOX3, BIRC32, JSRP1, NNMT3, NFKBID1,
CCDC141, PAIP2B, ZNF171, SRP14-AS1, PRKCB1, DENND1C1, HSH2D1,
LRMP2, ARID3A1, LRRK22, ATF7IP2, IL27RA1, IGHA11, RASGEF1B2, ZDBF21,
FSCN1, SVIP1, MALAT12, RHOH3, RP11-138A9.12, TAGAP3, RAB11FIP12,
SLC25A4, STK43, UTP20, TXN2, CD862, LINC004262, FMNL13, REL4, RGS102,
CYSLTR11, IDI12, LMOD31, MALT13, INTS63, NCF11, H3F3B2, SIPA1L11,
GPSM35, AKAP91, HMHA12, IGLL51, MARCKSL11, PPA11, PIM22, CKLF3,
TSC22D33, DUSP54, HSP90AA13, UCP24, KMT2E2, ODC11, CHCHD101,
RBM383, FAM107B3, DUSP22, ANXA65, LSP12, IER21, RSRP12, SOCS12,
VPS37B3, IDS2, TNFAIP83, KLF62, JUN3, SELK2, HSPH12, ARID5A2, and
DNAJA12.
Cluster 13:
CCL211, TFPI1, SCN3B, EFEMP11, ECSCR.11, SDPR1, RGS162, GNG111,
CLDN51, GYPC2, MYZAP, SNCG1, SLC24A1, C2CD4B1, HSD17B2, FABP4,
PCAT191, ANGPT22, KANK31, RHOJ1, RAMP21, OVOS2, IGFBP72, NR2F22,
TIE11, PDPN2, ADAMTS15, CCND12, HYAL21, ITIH51, FAM174B1, PTPRE2,
TBX1, AKAP121, HEY1, MIR99AHG1, TUBA1A3, ROBO41, PCSK6, MYCT11,
TSPAN111, WFS1, CD91, VIM2, B3GNT7, KLHL4, DNAH5, F2RL31, NOVA21,
CRIP21, SLCO2B11, MRC11, PDLIM11, S100A104, LINC009362, CD2001,
SLC22A23, TM4SF11, RCAN11, HSD3B71, ADIRF2, EME21, PIM32, EGFL71,
LINC00176, CTD-2553L13.10, CXorf361, APP2, SPTBN11, PINLYP, CEACAM1,
CDHR3, RAPGEF51, FLT4, CTC-425F1.4, EPB421, LAYN1, CHMP4C,
SLC25A23, B3GNT8, MTUS11, ELK31, LINC008391, FAM212B1, TSHZ22,
KDR1, EFNA51, TPPP, KRT182, PBLD2, TPCN1, GS1-124K5.3, MIR3142HG1,
RP3-395M20.122, CTSH2, TM4SF181, FXYD21, PKIG2, EPB41L4A1, LYPD5,
ZMAT32, CNIH3, CDC42EP21, MEDAG2, PLPP11, CD592, LRG1, TCTN3,
AKR1C31, AC074289.11, NOV1, DDIT31, ADCY41, CAV12, ARL4A1, C17orf80,
TC2N1, AFAP1L11, SH3BGRL21, KLF63, LMCD12, PTPRN21, EXOC3L11,
PALD12, IFITM21, KBTBD21, IGF12, GRAP3, TMEM255B1, LMO21, CYP39A1,
ARHGAP291, ACKR3, FXYD62, PHACTR22, ANKRD23, ZDHHC1, PEAR11,
ITGA8, TRIOBP1, SMAD11, ARHGAP271, LRRC322, ZSCAN181, TRIB13,
GADD45B3, PADI3, EDN11, KIF3C, ZNF4671, NRP22, LAMA44, NFKBIA2,
TSPAN71, TMEM117, HOXD81, ZNF8311, CDH51, CCDC1411, FSTL33,
TGFBR21, RP11-115D19.12, CDKN2A2, ADAMTS5, SRRM31, EDNRB2,
AKR1C11, IQCA1, SIN3B, MAFF2, PROCR2, NFATC12, GABRE, SPIN4, OLFM1,
MAP1LC3A3, LEF12, RAI142, GIMAP72, HSPA61, CDH132, NFAT51, YES11,
SORBS1, NFKBIZ3, CHST15, TSPAN122, CNKSR31, ZNF772, ZFP30, SPIN2B,
RP11-108M9.41, HYKK, MRAS1, GUCY1A32, KCNN31, CXCL22, DUSP61,
TSPAN5, HHEX2, RP11-53O19.3, PPARGC1B, PTPN3, PCDH171, ARL6IP12,
ZNF33B, MPP7, HSPA1A2, GAS63, NFIB2, NOX41, FAM43A2, GIMAP41,
CRIM11, KLF22, CCL24, LRP5L, MAF1, CEP192, COL4A5, ATF33, FAM171A11,
EFNA12, SLC35E42, TLR41, NR2F1, OLFML2A2, RP11-242D8.1, SAMD101,
CRIP14, EHD1, TTYH31, CITED41, TPPP32, HES14, FAM184A1, DAAM12,
KATNBL11, PPP1R15A3, SEC14L2, TIMP34, SYNJ1, CCDC191, ZFP364,
BMP21, RPS6KA21, PGM2L1, CYR613, IER51, DOCK5, SLC44A31, SDC31,
CNTNAP3B1, SEMA4C2, WDR35, FOS3, SCARF11, SHISA2, LDB21, MYC2,
CYB5D1, NUDT42, TSPAN151, TIMP14, PPM1F1, DYSF1, SRGAP11, NR1D11,
NFIX2, ZXDA, APLP23, ZFYVE162, ID11, PSMG3-AS11, VAMP52, ADAM102,
TAF5L, GATA21, KANSL1L, UNC5B1, TSPAN181, CCDC71L2, PTPN141,
DKK34, MDK3, LINC001522, KLF41, NXN1, PVRL21, ALDH22, UGP22,
MIR222HG1, HSPA1B3, ZNF500, ITGB41, CDK5RAP2, TGM21, JUN4, FMOD1,
SOX42, AKR1C2, ARID5B4, IL6ST1, GJA12, NEAT12, TUBA1B3, SERPINE12,
UACA2, CYB5D2, CALR1, RHOB3, ARHGEF122, YPEL21, MGP3, HMGB12,
FBXO25, CTD-3252C9.43, ITGAV2, HSP90B12, SERPINB12, PRKCDBP3,
ARRDC31, MIR4435-2HG2, HSPA52, and WARS1.
Cluster 14:
TK13, RCSD13, THBD1, CKLF4, GYPC3, TXN3, RRAD3, NUCB24, CD632,
TMPO2, TNFAIP84, TUBA1A4, CD441, LGALS31, ACOT7, HSP90AA14, SEP64,
EHD11, PLIN22, EMP33, RGS103, TMSB4X3, MARCKSL12, HSPA53, DDAH23,
TGIF13, and MYADM3.

Fibroblast Clustering Identifies Unique Sub-Populations Associated with Response and Resistance to Immunotherapy

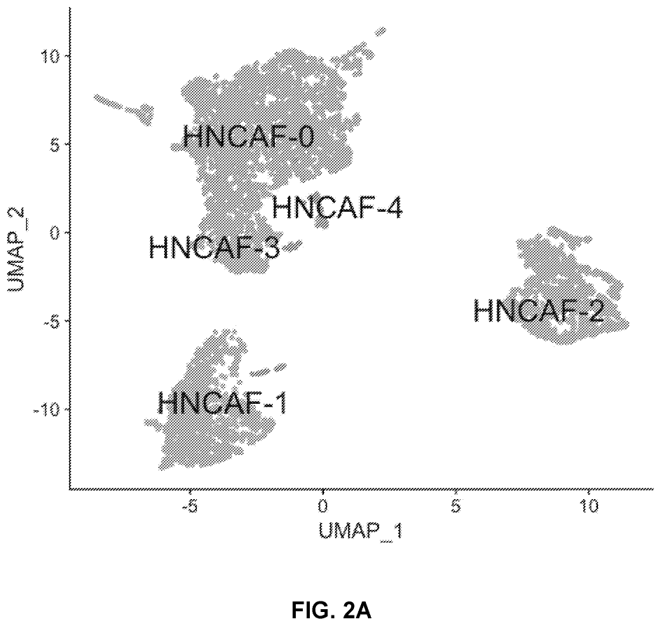

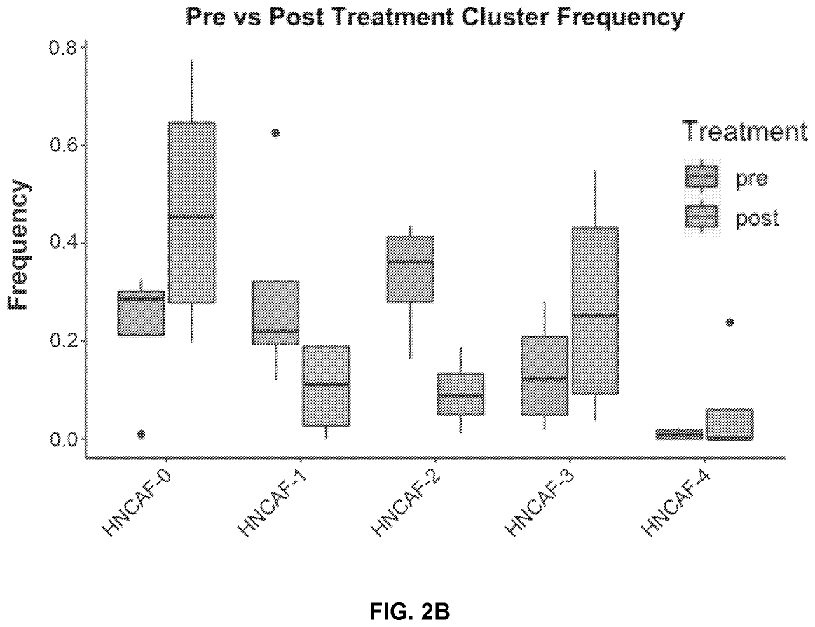

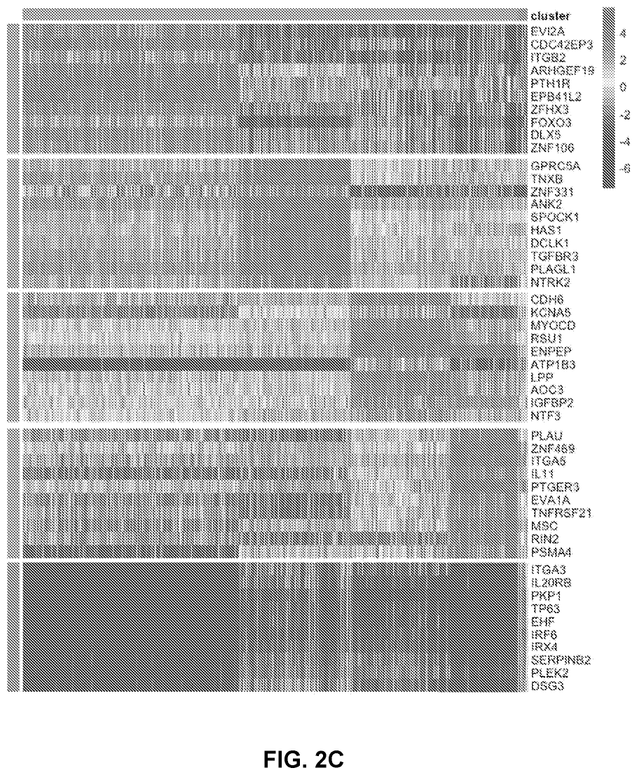

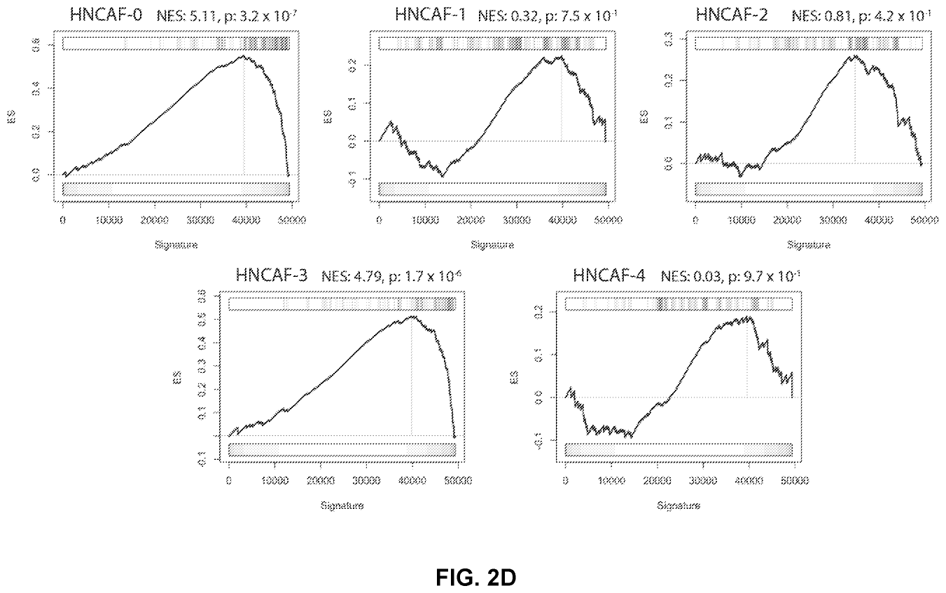

To further evaluate functional differences between the distinct CAF sub-populations in the HNSCC TME, protein activity-based sub-clustering focusing only on fibroblast cells was performed. The analysis identified five molecularly-distinct CAF clusters termed HNCAF-0-HNCAF-4 (FIG. 2A), which exhibited equally distinct fractional cell representation changes following treatment. Specifically, cell fractional representation increased in HNCAF-0 and HNCAF-3, decreased in HNCAF-1 and HNCAF-2, and was unaffected in HNCAF-4 (FIG. 2B). The top ten most differentially active proteins in each of the five clusters helped further highlight their functional properties (FIG. 2C), by presenting the unique signature of each subpopulation and a ranked list of differentially active transcription factors and signaling molecules, which help define the biology of each HNCAF phenotype (Tables 2A and 2B). To assess the associations of each CAF subpopulation with response to αPD-1 immunotherapy, bulk RNA sequencing profiles from the 40-patient parental cohort annotated with clinical response were analyzed. For this purpose, VIPER was first used to generate protein activity profiles from each bulk profile, using fibroblast specific regulatory networks generated at the single-cell level, and then the enrichment of the most differentially active proteins in each HNCAF subpopulation (marker protein sets) in proteins differentially active in responders vs non-responders was evaluated. The analysis revealed statistically significant association of HNCAF-0 and HNCAF-3 in pre-treatment samples of patients who subsequently responded to immunotherapy (FIG. 2D). This data suggests that the HNCAF-0 and HNCAF-3 populations, which also expand following nivolumab treatment, are highly predictive of favorable response in human HNSCC patients. In contrast, HNCAF-1, HNCAF-2, and HNCAF-4 cells did not expand following therapy and their markers were not significantly enriched in responders vs non-responders. Critically, the same analysis using clusters inferred by gene expression analysis could not identify any differentially represented OAF population.

TABLE 2A
Differentially Activated Proteins for each HNCAF cluster shown in FIG. 2A

Cluster 0: EVI2A, CDC42EP3, ITGB2, ARHGEF19, PTH1R, EPB41L2, ZFHX3,
FOXO3, DLX5, ZNF106, GPC1, PIK3R1, FGFR2, TNFAIP8L3, PTPRD, ZFHX4,
LIFR, TCEA3, ITGA10, NUDT4, EPHB2, AK1, PTGIS, ALPL, FNDC4, NR3C1,
MKX, MARVELD1, HERPUD1, ITGB5, FZD1, GJA1, ANXA4, ENPP2, PTN,
CNKSR3, COLEC12, PRMT2, ANKH, SMOC1, ENAH, PDGFC, ATRAID, CADM1,
VASN, FGF7, LSP1, SLC29A1, ARL4C, SH3KBP1, HOXB2, SVIP, CHD9,
TCEAL2, RERG, PSIP1, NRP2, BTG1, CDC42BPA, STXBP6, CAPN2, HLA-
DRB1, DDIT4, TOB1, EVL, RUNX2, CREM, ARL2BP, NOTCH2, RGS1, TERF2IP,
ZBTB16, DAP, FXYD6, DUSP2, TSC22D3, ITGA11, HLA-DPB1, SYTL3, HLA-
DQA1, TRAT1, RBM38, CCL5, SMAP2, HDLBP, STK17B, SH3BP5, ARRDC2,
STEAP4, SLA, GSPT1, IKZF3, ELF1, HLA-DRB5, SH3BGRL, APBB1IP, TXNIP,
CTNNB1, MAGED1, NTRK3, CDH11, CD2, ITGB1BP1, ID2, EEF1D, CAMLG,
SLC44A1, RASSF5, PTPRC, VPS37B, PASK, DPYSL3, CXCR6, GPR65, HLA-
DQB1, HYAL2, CD8A, CD8B, DTHD1, RUNX3, FGFR1, PIK3IP1, DDAH2, CLMP,
CXCR4, CPE, PDCD4, CNIH1, ARHGAP30, CD3E, FYN, LCK, HCST, SDC2,
GZMB, LPAR2, CD3D, SH2D1A, TLE4, S1PR4, KLRC2, IL6ST, CFB, AKNA,
ICOS, CD3G, INPP5D, TBC1D10C, ZEB1, GPR171, JAML, HLA-DPA1, CD7,
SKAP1, CD96, ETS1, UNC5C, CXCR3, CD53, JADE1, IL10RA, LRP1, SP100,
KLRD1, RHOD, CD247, PPP2R5C, GATA2, FNBP1, IL2RB, CBLB, EZR, ID3,
SH2D2A, CD27, ARF5, LAX1, BCL11B, ZFP36L2, IKZF1, GSN, TNFRSF18,
EPB41, SIT1, CCL4L2, GZMA, MTUS1, TSPAN4, AGT, FOXC2, CDKN2A,
HSPG2, PRF1, SLA2, CORO1A, WSB1, ZFAND5, CD74, CTLA4, MMP2, CPNE7,
PDCD1, S1PR3, HNRNPDL, GAS1, CCNH, SPOCK2, PLSCR4, IL2RA, CD48,
S100A10, RORA, KLF9, ZBTB20, RASGRP1, ITGA4, CCR7, ITM2C, SFPQ,
SKAP2, RGS3, DUSP4, CTSZ, PPP1R2, RHOH, SOCS1, RRBP1, SMOC2,
LGALS3, KIR2DL4, SRSF2, SLC41A2, PTP4A2, CDKN2B, HLA-DRA, STK4,
KLRC1, ANTXR1, APP, RGL4, CREB3L1, MGST3, GRAP2, CD5, C2orf88,
CERCAM, MORF4L1, PSCA, NDFIP1, TCEAL4, LBH, CRYAB, GPR132, CAPS,
SLC38A5, CYBRD1, ARID4B, TMEM204, MEF2C, CPM, ADGRL4, ACAP1, DST,
ITGBL1, EID1, SLC7A5, DSG1, BASP1, TANK, CRABP2, ZNF428, C18orf32,
UTRN, PITX1, ASH1L, CLEC10A, RBPJ, IFITM2, NFIX, CSF2RA, ESD, PINK1,
CD99, FCER1A, GRIN2A, FHL1, PPP1R10, APOLD1, ADRA2B, GLIPR1,
ARHGAP15, HDAC7, PDGFRA, HPGD, GPR183, IFI16, WWTR1, CREB3L2,
IL2RG, CSDE1, FBXW7, PRRX1, ATP6AP2, ANXA1, SEMA7A, MAFB, MYC,
KLF2, FOXC1, AXL, LTB, ABCC9, CD24, RAP1B, JMJD1C, TNFSF12, MAPK13,
RPS27L, NDN, HLA-DOB, GNG7, SDC1, CLU, NET1, VCAM1, PBX1, PPIC,
RABAC1, CALML5, CD9, RGS10, GPR157, RHOJ, SELP, RAMP3, ACKR1,
SLCO2A1, TMEM59, HLA-E, CAPNS2, RHOF, PALLD, NEO1, ZNF296, GDI2,
WASF2, CLEC4A, DUSP1, SPIB, TGFB1I1, HLA-DOA, CD1C, EMP1,
RASGEF1B, EMP2, PNRC1, PEBP1, CEACAM6, NSG1, ARF4, HEXIM1, STX11,
FOSL2, INTS6, NDRG2, PRDX4, MEOX2, RAB32, EMP3, EIF5, VOPP1, JUN,
ARL4A, SOX18, PDE4B, ATP2B1, FAP, IFNG, TWIST1, S100A6, DAPP1,
FCER1G, AKAP13, UBE2B, PRSS27, DDR2, EBF1, RAB30, KLRB1, PLEKHO1,
VAMP2, RIPK2, RAMP2, HLA-B, CCR6, RAB33A, GNAS, ITM2B, CTSH, ETV3,
TSPO, NOTCH4, DERL3, YWHAQ, SKIL, REL, FPR1, BATF, SDCBP, TGFBI,
CEBPB, GPBP1, THY1, SAP18, NR4A3, TRAF1, VASP, ATF6B, JUNB, TSHZ2,
F2RL3, BTG2, RAB31, KCNMA1, LPL, UBC, SOD1, DDX5, TRIM22, RPSA,
PFDN5, RASSF4, BCL2L11, FCGR2B, MARCO, DNAJB6, SPRED1, TYROBP,
RASSF7, MYADM, LY96, LY86, C3AR1, LCP1, MS4A6A, ADAP2, ARID5B,
SERBP1, and STAB1.
Cluster 1: GPRC5A, TNXB, ZNF331, ANK2, SPOCK1, HAS1, DCLK1, TGFBR3,
PLAGL1, NTRK2, RBM39, DPYSL2, RHOB, LRPAP1, GLDN, GAP43, PROS1,
EPHB6, STC1, CXCL14, MTDH, C2CD4B, F10, CD81, FCGRT, FEZ1, KLF4,
NFIA, CEBPD, CDKN1A, ATF3, APLP2, LEPR, NR4A2, NR4A1, EGR2, FXYD1,
JUND, RALGAPA2, FOSB, GADD45B, CD55, ATP1A1, SERTAD1, FABP4, RTN4,
NFKBIA, PLIN2, HLA-DQA2, RND1, AHNAK, DLC1, AQP3, C5AR1, ABCA8,
IGFBP6, TLR2, TACSTD2, WLS, SLC38A2, DZIP3, STAT3, SLC11A1, NLRP1,
CYSLTR1, SLC16A10, MAFF, TREM1, PIM1, LILRB2, IGF1, TSC22D1, DLL1,
GPC3, SPTBN1, SIGLEC1, TBC1D4, LYPD2, KLF10, PLPP1, PRNP, LY6D,
TGFBR2, TNF, CD200, EGR1, CSF2RB, BEST1, CYBB, AKAP12, OLR1, LCP2,
TRIB1, CCRL2, CD151, EREG, AQP9, GABRP, THBS1, HBEGF, CD163, AOC1,
DNAJA1, SPARCL1, AQP1, CLU1, CCNL1, SLC40A1, VEGFA, ANXA11, CLTA,
SELE, GRB2, IL1B, S100A8, HES1, IGF2R, LITAF, NFKBIZ, TXN, PMP22, ARL2,
ZNF267, CCL18, YBX1, PLEK, CD14, LILRA1, ICAM1, SPRY1, IL1RN, CSF1R,
ITGB6, MSR1, S100A9, SDC3, GNB4, KRT19, TTYH3, TREML1, CLTB, GSTP1,
PECAM1, CXCL2, AHNAK2, CD300E, LAMP1, THBD, RNF20, ADORA3,
FCGR3A, SFN, HSP90AA1, XBP1, LIMS1, MS4A4A, KRT5, IL6, S100A12,
KRT17, CMKLR1, SOCS3, ZNF385D, HRH2, PPP1R15A, LAIR1, CREG1, PBX11,
CD68, MPP1, MS4A7, GPR34, CSRNP1, NDRG1, LILRB5, CLEC7A, ZFP36L1,
GADD45G, NEU1, C3, SCN3B, TENM2, HCAR3, BHLHE40, DNAAF1, FOS,
ETS2, LAT2, DYNLL1, MCF2L, TMEM591, ZNF706, PLPP3, HSPA5, SDC4,
CD79A, SULF2, KCNMA11, FURIN, KLF6, CITED2, PRDX1, PRSS12, JAM2,
TGFB3, SDCBP1, LHX8, TCF4, HIST1H4C, ADRA2A, HES4, ADM, SLC7A1,
VAPA, PLP2, ADAMTS1, IGFBP3, PLSCR1, ZEB2, ZNHIT2, MCM7, BRCA1,
F2RL2, CYBRD11, PTTG1, RAMP1, ASCL2, SGIP1, YWHAZ, AGPAT2,
TNFSF13B, IFITM1, GJB2, EIF4EBP1, ACKR4, DSC2, PROCR, PDIA3, NCOA7,
WWTR11, AP2A2, CD91, FSTL1, GNAI2, SLC7A7, CADM3, CD83, EHMT1,
M6PR, OSM, GRHL3, ARHGAP18, SOD2, IL1R2, BLVRB, ARHGAP29, PILRA,
RHEB, ITGBL11, ARID5A, HMOX1, FPR3, ACKR11, FOSL1, SLC9A6, FCHO2,
VAMP8, TNS2, RHOV, ARID5B1, CD4, PLEC, GPRC5B, SLCO2B1, DDIT3,
SNX6, CD40, TNFSF10, RDX, ZNF605, PCDH17, CPNE1, RAB5C, C3AR11,
JUP, ABCA1, TGOLN2, FGF10, NOSTRIN, MATK, SIAH2, KLF21, SIGLEC10,
SFRP1, HSPA8, SP5, GPR162, FLT3, FHL11, TNFSF121, TREM2, FCGR1A,
MAP1B, ZMAT1, NR1H3, EHD1, TSPAN8, LPAR6, HDAC71, EMP21, CXCL16,
ERN1, LILRB4, ABCC91, APLNR, ANKRD1, NOS3, CDH5, ADRB2, IL1A, GLMP,
AGTRAP, MRC1, FCGR2A, TNFRSF1B, SECTM1, CLEC4E, ATF4, DAB2, KRT1,
RB1, MERTK, RALBP1, ATP6V1G1, ARC, SOD11, PLXND1, CD44, MYADM1,
MEOX21, TSHZ21, CNIH4, TSPO1, RRAD, IGSF6, GRN, BSG, RHOG, SLC7A11,
STMN1, CD84, DDX51, BTG21, CD209, ZNF4281, CCL13, DIRAS3, EIF51,
NFE2L2, F3, HSPD1, PDK4, NFIC, IRF1, CHD3, DIO2, MXD1, CD69, GEM,
PCBD1, PKIG, SLC1A5, TGFBI1, RAMP21, NFIB, WASF21, CAPNS21, ACHE,
LY961, STEAP2, GNAS1, SPRED11, JUNB1, SLIT2, SERPINE1, CD300A,
TAF10, ADAP21, JUN1, ITM2B1, MAFB1, SHOX2, MARCO1, ANXA2, ESD1,
ERO1A, GPX1, TFPT, TNFAIP3, CD274, FCGR1B, ITSN2, PARK7, CD79B,
GYPC, STAB11, NRK, PPFIBP1, KCNK2, JMJD1C1, VIM, RAC2, HCAR2,
CSNK2B, CAMK1, CD991, FCGR2B1, NDN1, LSR, NR4A31, ADGRE5, CBX6,
CD164, CALML51, SLC9A3R2, MS4A6A1, BAG3, NFKB1, MNDA, TMEM219,
NLRP3, PAX3, SGK1, PITX11, IFNG1, LIMA1, ITGB4, SPI1, PEBP11, CLEC2B,
CCL2, PTGER4, NGFR, LPL1, NAMPT, LAG3, MAP3K8, ANKRD11, NFIX1,
NCOA4, RND3, IL15RA, ANK1, SLC24A4, RHOA, KMT2E, and DST1.
Cluster 2: CDH6, KCNA5, MYOCD, RSU1, ENPEP, ATP1B3, LPP, AOC3,
PPP1CA, RAC1, YWHAB, CCL26, KCNMB1, TRAF4, PTPRG, EMX2, CAP2,
HMGN1, HSPE1, PPP1R12B, YWHAE, ITGA6, BNC1, HSP90AB1, KIAA1324L,
KDR, MYO1B, ID1, JAG1, RGS5, CYCS, CD36, PTK2, STRAP, ACTN1, RAN,
SLIRP, FLT1, ESAM, EPAS1, FKBP1A, CAP1, ANGPT2, ZNHIT1, CAV1, SUB1,
C1QBP, ARHGDIA, AFAP1L1, EDNRA, SEP7, SLC2A3, SOX15, ADAMTS11,
RAB10, PDCD6, NOTCH3, RAB38, TRPC6, HMGN2, YBX3, ADRA2A1,
RASGRP3, BZW1, PLCB1, CLIC1, ILK, UBE2D3, HMGA1, PA2G4, GAPDH,
CD34, PHB, TFAP2A, GGT5, PRMT1, EFNB2, VAPA1, CBX3, PDLIM1, BMP2,
ARF1, ZNF7061, ID4, POLR2L, MSN, RDX1, HSP90AA11, MYBL2, PRSS121,
HES41, TLR10, MYO10, SQSTM1, VDAC1, BAX, NRP1, PCDH171, NOTCH41,
PKM, FURIN1, GPR1621, CAV2, FLNA, ACTB, DYNLL11, HLA-F, FOXM1,
NEDD8, BDKRB1, SNAI2, PRDX11, PDGFRB, HMGB1, ANKRD111, FRMD6,
GNG5, RANBP1, PTPRE, JAM21, CHMP5, SCAND1, PLPP31, NDUFA13,
TXNDC17, HSPB1, ETS21, GNB1, ENO1, ADGRL41, UBE2L3, YWHAZ1, FSTL3,
PRKAR1A, CCND1, CALM1, NME2, ZNF6051, HCAR21, ZFP36L11, SPRY11,
EPS8, GPM6B, SYTL2, CDH51, EHD2, SLC7A12, UBB, IL1R21, TNFRSF12A,
CEACAM1, ARPC2, CDC42, NEDD4, SLC3A2, PDIA6, ANK11, NTN4, IGFBP2,
PLEKHO11, MRGPRF, FOXS1, PRDX2, NFE2L21, HOPX, ARID5A1, RALBP11,
KRT191, PINK11, HDAC72, PPT1, CALD1, NOS31, PYCARD, ASCL21,
TAX1BP3, TNS21, PFN1, ARHGAP291, ZMAT11, ZEB21, FOSL11, EDNRB,
PRC1, NTM, ARNTL2, PLXDC1, PIM2, DIRAS31, VOPP11, WNT6, TNFSF13B1,
IFI6, CDCA5, NTF3, CD59, YWHAH, CFL1, RGS16, TFPI, ANGPTL4, ASF1B,
ARHGDIB, HLA-A, APOE, CALR, APLNR1, ARF6, DUSP6, RAB13, ITGB41,
SLC9A61, FCHO21, ZWINT, PECAM11, CENPU, MIF, ENG, ATF41, LYPD21,
BHLHE41, PLAT, HINT1, CD177, MCM71, P2RY14, IFNGR1, MNDA1, GTSE1,
DAPP11, NOSTRIN1, SECTM11, SLC9A3R21, SPIB1, FANCI, BCAM, HSPA81,
ENTPD1, ORC6, CXCL10, NDC80, RASSF41, CD1E, CD300A1, SOX181, MBP,
CENPF, RHOBTB1, LSR1, CD47, FHL2, HSBP1, SSTR2, LRRFIP1, NRG1,
CCL131, CBFA2T3, CCR61, LY6E, IFITM3, NDFIP11, TNFAIP6, LYPD1, SIAH21,
YBX11, SAP181, LMO4, PLEC1, CSNK2B1, HMGB2, CD401, BRCA11, RHEB1,
PRCP, TAF101, FST, NEU11, SEMA7A1, AURKA, RAMP11, PLP21, PROCR1,
CEACAM61, IGFLR1, STMN11, KCNK6, IRF4, IFI27, GDI21, APOLD11, HLA-C,
DRAP1, CASP1, INSIG1, PARK71, S100A11, ITSN21, AVPR1A, LAG31, HLA-
DOB1, SERPINE11, CD1511, STAT1, CD1C1, KRT11, ADRA2B1, FPR11,
HCAR31, LPAR61, TREML11, IGSF61, NPM1, PTPN1, GRIN2A1, NET11,
EBF11, RHOG1, BST2, B2M, LAT21, CTNNAL1, LGALS3BP, IL1RAP, AES,
CD2091, CD79B1, HRH21, YWHAQ1, FCER1A1, MARCKS, UACA, BCL2L111,
ADORA31, CD79A1, RERGL, VAMP5, TMEM2191, CHN1, MATK1, NCOA41,
LCP11, OSM1, REL1, SIGLEC101, MGST2, RB11, VAMP81, POU2F2, SHOX21,
CD41, SPI11, AP2B1, ARHGAP151, TNFSF14, ITGA8, KCNJ8, TSPAN15,
MERTK1, FCGR1B1, CD841, MYO1G, CXCL161, ZNF2961, GNAI21, DERL31,
KMT2E1, RHOA1, SHISA5, BIRC3, VASP1, and SLCO2B11.
Cluster 3: PLAU, ZNF469, ITGA5, IL11, PTGER3, EVA1A, TNFRSF21, MSC,
RIN2, RGS4, TSPAN9, PRDM1, PTK7, ITGAV, PON2, PLAUR, SOX11, ITGA1,
TWIST2, SULF1, WNT2, HIF1A, PLPP4, CDC42SE1, PTGES, APBA2, STEAP1,
B4GALT1, SLC39A14, STAT2, LAMP5, ECM1, MSX2, MYH9, ITGB1, LMCD1,
ACKR41, CLIC4, RBPMS, CHST11, PMEPA1, PARP14, SGIP11, SCG5, AP2S1,
TNFRSF1A, CHIC2, GRK5, RAB1A, DPP4, INHBA, F2RL21, F2R, WNT5A,
GREM1, IL15RA1, CKS2, SOX4, LOXL2, MMP14, NTM1, TNFAIP61, ADM1,
LY6E1, B2M1, LHX81, SNAI21, CEMIP, EID3, FOXP1, CHN11, CCL8, ANXA5,
ADAM12, AGTRAP1, IGF2, DIO21, CNIH41, IFI271, CHP1, CYBA, PDGFRB1,
IGFBP31, PDPN, STEAP21, IRF7, ACKR3, CCL21, TGFB31, RHOC, HIVEP3,
CHMP51, HM13, GEM1, SLC2A6, FSTL11, ABL2, GLIPR11, RABAC11,
CHMP4A, CD63, HLA-C1, RAP1B1, LMO41, GABARAP, CALD11, CALR1,
HSPA51, PKIG1, SULF21, HLA-F1, TSPAN151, SCAND11, C31, PLXDC11,
PDIA31, LGALS1, FAP1, CD591, ZNF503, DDR21, ANGPTL41, PLAT1, PRDX41,
IL1RAP1, GPM6B1, ARF41, AP3S1, THY11, TRIM221, PALLD1, NDUFA131,
BMP21, LYPD11, MARCKSL1, TGFB1I11, PDIA61, PLK2, HLA-B1, IFITM31,
GNAI1, PTGIR, BATF3, NFE2L3, RAB321, ATP6V1G11, MX1, RND31, CXCL3,
BST21, CD1641, PTPRE1, SHISA51, PFN11, BSG1, EDNRB1, RAP1A, EDF1,
GGT51, PSMA4, PRRX2, SGK11, PAG1, LGALS3BP1, NOTCH31, CALM2, F31,
LPXN, TNFRSF12A1, HLA-A1, PPIC1, RAB131, FRMD61, KCNK61, GNG11,
CLIC11, RRAD1, TAX1BP31, CERCAM1, CLEC5A, AVPR1A1, TNFAIP31,
RAB33A1, IL7R, TNFSF101, CXCL101, CBX31, MORF4L2, DRAP11, CDC421,
DUSP61, LGALS9, MARCKS1, GNG2, PHLDA1, BHLHE401, IL10, AXL1,
PDLIM11, ZNHIT11, C18orf321, IFI61, PMAIP1, EFNA1, LY6K, ARNTL21,
FKBP8, ARHGAP152, CREB3L11, UBB1, IL1R1, ANKRD12, SERPINE12,
CTNNAL11, RHOBTB11, AP2M1, NEDD81, SLC3A21, ATP1B1, CD402, DLL11,
BASP11, ARC1, MIF1, CD471, IGFLR11, SLC7A51, VAMP51, BIRC31, ENY2,
CD441, VCAM11, NEDD41, RGS31, ARF11, CXCL21, SDC41, PARK72, RAB101,
IGFBP61, PRKAR1A1, SDC11, NAMPT1, PRMT11, CD1771, PLSCR11, PTHLH,
CAV11, ANGPT21, RAB311, STAT11, AP2B11, CFL11, IFITM11, ENTPD11,
PTEN, THBS11, NME21, SUB11, FOXS11, S100A111, UBE2L31, NRP11,
TREM11, F2RL31, IL61, GNG51, TANK1, ACTB1, NCOA71, ADD3, MYO1G1,
CRABP21, CPNE11, CALM3, FST1, PROCR2, CD82, NEO11, VAMP21,
CD300E1, PKM1, PFDN51, IL1R22, ANTXR11, BAX1, IL1A1, GYPC1, RASD1,
SOD21, KLRB11, HPGD1, FLNA1, TCF41, SIGLEC102, TRAF11, TNFRSF1B1,
HCAR22, PRCP1, CHD31, RRBP11, GPX11, ARPC21, SLC1A51, POLR2L1,
EDNRA1, HSBP11, KCNJ81, MGST21, EMP11, S100A121, SERPINB9,
AGPAT21, CAV21, YWHAH1, TWIST11, RBPJ1, NLRP31, GNB11, LTB1, KLF61,
S100A61, IL2RG1, AES1, TXNDC171, TNF1, LSR2, RGL41, UBE2D31, ILK1,
SYTL21, ENG1, ACAP11, THBD1, MAP1B1, EHD11, SQSTM11, IFNGR11,
CAP11, SLC2A31, LILRA11, CCL4L21, GAPDH1, BDKRB11, VDAC11, NGFR1,
S100A16, SLC41A21, GPR1831, ACTN11, BZW11, IFITM21, ADRB21, ACTG1,
CCRL21, RAB5C1, RHOH1, NACC1, PRRX11, NRG11, PLPP32, ETS22, SLA21,
HCAR32, RANBP11, ITGB61, TRIB11, IL2RA1, PLP22, IL1B1, SELE1, CD831,
CD51, XBP11, KLRC11, FCER1G1, FOSL21, AQP91, TGIF1, ANKRD112, PIM21,
PPP1R21, ITGB42, PTPN11, MSN1, CFLAR, RHOF1, ATP2B11, LIMA11,
SLC7A111, PLEK1, ZNF2671, IRF41, ETV31, BATF1, SKIL1, HRAS, RHEB2,
CSF2RB1, ASH1L1, LCP21, AHNAK21, INSIG11, STX111, EREG1, HES42,
CD1E1, ENO11, MYADM2, ICAM11, GPBP11, OLR11, APOE1, ZNF2962,
RAB301, CASP11, RASGEF1B1, SLC24A41, HBEGF1, TFPI1, FPR12,
TNFSF13B2, ACHE1, KIR2DL41, RHOG2, UBE2B1, MYO101, RGS2, CCR62,
HINT11, PLEC2, NFKB11, CSNK2B2, TLR21, CSF2RA1, CBFA2T31, SECTM12,
DNAJB61, FCER1A2, LRRFIP11, VEGFA1, UACA1, VASP2, RIPK21, CD79A2,
MXD11, REL2, CD2741, CLEC2B1, ITSN22, ERO1A1, AKAP131, NET12,
PTTG11, VAMP82, FCGR1B2, TNFSF141, LIMS11, RGS161, MAP3K81, FLT31,
PILRA1, C5AR11, SLC9A3R22, LILRB21, GPR1571, CLEC7A1, TSC22D11,
ATP6AP21, TMEM2041, GRN1, and RPS27L1.
Cluster 4: ITGA3, HRH22, IL20RB, GJB21, TFCP2L1, FGFBP1, TXN1, PKP3,
PKP1, S100A81, DSP, IL1RN1, CLTB1, TPD52L1, TP63, EHF, CA2, NACC11,
CLCA2, TCF19, ARNTL22, SFN1, IRF6, IRX4, KRT51, S100A91, DSC21,
TACSTD21, CD79B2, TRIM29, ATP1A11, DSC3, PRSS122, SERPINB2, HRAS1,
PLEK2, S100A14, GSTP11, RALBP12, ANXA3, PKM2, LY6D1, MYBL21, ENO12,
GAPDH2, CD821, JUP1, UBE2C, DSG3, IMPA2, HMGA11, HLA-DQA21,
CD2001, KRT171, IFITM12, KRT192, YWHAZ2, MIF2, TOP2A, GRHL31, ETV4,
PTTG12, ZWINT1, LAT22, EIF4EBP11, NDRG11, FANCI1, ADM2, C1QBP1,
RHOV1, TENM21, AGPAT22, BRCA12, TFAP2A1, FOXM11, SLC24A42,
SOX151, NPM11, CENPU1, MCM72, FSTL31, SLC1A52, CDCA51, RANBP12,
PA2G41, HIST1H4C1, UBE2L32, BNC11, PHB1, YBX12, RRAD2, PLEC3, KIF14,
SERPINE13, FABP41, ORC61, HMGB11, TXNDC172, CD403, ADRB22,
HCAR23, ATF42, HMMR, ASCL22, ASF1B1, PLP23, SLC7A112, ARHGDIA1,
SLC7A13, FOSL12, DYNLL12, PLSCR12, HMGB21, HINT12, RAN1, PRC11,
CXCL141, AHNAK22, ERO1A2, PLK21, RAB102, LPL2, S100A112, HSPB11,
HSP90AB11, YWHAE1, CIB1, LRRFIP12, PDLIM12, ITGA61, ZNHIT21, LY6K1,
SELE2, GTSE11, HSBP12, PRDX12, RAB381, S100A161, STMN12, LSR3,
NCOA72, CPNE12, MYO102, YBX31, HSPE11, NEDD42, BEST11, ZNF7062,
CD1512, FPR13, RHEB3, AP2M11, MORF4L21, HES43, POLR2L2, CAP12,
RND11, GNG52, GPR1572, ACTG11, RIN21, PSMA41, HMGN11, SDC42,
ATP1B11, SGK12, PPP1CA1, CXCL31, AGTRAP2, PRNP1, RND32, SERBP11,
TLR101, RALGAPA21, ETS23, NME22, CENPF1, NEDD82, KRT12, ANKRD13,
FURIN2, VDAC12, ITGB62, MSX21, PFN12, IFI62, THBD2, DRAP12, SLIRP1,
TWIST21, CFL12, EVA1A1, CBFA2T32, TGFB32, CAV12, AURKA1, PTK71,
GNB12, FKBP1A1, MBP1, CYCS1, LY6E2, IL1R23, ITGB43, CKS21, GNAI11,
BHLHE402, SFRP11, TRIB12, EMP22, ATP1B31, PDCD61, RASSF71, UBE2D32,
IFITM32, HIF1A1, NFE2L22, SGIP12, BZW12, TRAF41, CD1C2, BASP12,
CXCL102, RBPMS1, SHISA52, ENY21, HSPA52, INHBA1, TNFSF142, NDC801,
MX11, CBX32, PRMT12, NFKBIA1, PRDM11, CDC42SE11, RNF201, VEGFA2,
SAP182, HNRNPK, SCN3B1, FLNA2, NGFR2, RHOD1, PROCR3, SIGLEC103,
IGFBP62, ACTN12, AP2S11, STRAP1, HMGN21, RAP1A1, DAPP12,
TNFRSF12A2, TGFBR21, RAC21, BST22, TSPAN91, RHOF2, CHP11, SHOX22,
MATK2, CLIC12, VAMP83, C2CD4B1, IL111, THBS12, EDF11, YWHAB1,
HSP90AA12, IL62, INSIG12, CHD32, ANGPTL42, CCL22, IRF71, LCP12,
SQSTM12, AOC11, SPIB2, PDK41, LILRA12, TSPO2, ATP6V1G12, ITGA51,
CALR2, CD79A3, CEMIP1, ACKR42, IGFBP32, SOD22, S100A122, LYPD22,
STEAP11, GABRP1, BATF31, F2RL22, PKIG2, SCG51, CCRL22, PRDX21,
CD472, ZNF4691, IL15RA2, CD300E2, HCAR33, CSF2RB2, PTPRE2, MGST22,
F32, ACTB2, PCBD11, LMCD11, CD442, IL1B2, DNAAF11, ZNF2963, AQP92,
FST2, BDKRB12, PDIA32, GAS11, TNFAIP32, EHD12, PARK73, ANXA21, TNF2,
MSC1, IL101, OLR12, PDIA62, TCEAL41, PTHLH1, DUSP62, HBEGF2, WNT21,
TRAF12, AQP31, ADGRE51, HLA-F2, IL1A2, STAT21, VAMP22, DIO22, and
VASP3.

TABLE 2B
Differentially Expressed Genes for each HNCAF cluster shown in FIG. 2A

Cluster 0: HTRA1, ASPN, COL1A2, COL1A1, COL3A1, LUM, MXRA8, GALNT1,
CDC42EP3, SPARC, CTGF, PLEKHA5, LSP1, BGN, MMP2, TSC22D3, EVI2A,
CTSK, COPZ2, PCOLCE, CDH11, COL12A1, NBL1, FBLN1, OLFML3, GAS1,
GJA1, ALPL, CLEC11A, CTHRC1, DCN, RCN3, COL6A3, ENAH, THBS2,
ZFP36L2, VIM, AEBP1, KDELR3, COL16A1, DDIT4, COL5A2, MDK, S100A13,
MXRA5, FMOD, COL5A1, OLFML2B, COL8A1, SDC2, CTNNB1, MARCKS,
LAPTM4A, PFN2, PRSS23, SDC1, ENPP2, SEPP1, PIK3R1, NRP2, DLX5,
CNKSR3, SERPINF1, FKBP10, CERCAM, GPNMB, MAFB, GOLM1, ANKH, MT-
ND5, KCTD12, FGFR1, FAP, IGHG3, ERRFI1, PRRX1, PDGFD, ANGPTL2,
P3H4, ZFHX4, MMP14, MFAP2, IGKC, BAMBI, LOXL1, SERPINH1, SH3BP5,
CLMP, FAM134B, MT-ND4, SFRP2, SSPN, LMO7, BICC1, PODNL1, VCAN,
VCAM1, SERTAD4, SCRG1, HSPG2, IGLC2, EDIL3, EFEMP2, ANTXR1, FSCN1,
GLT8D2, PDGFC, STK17B, IGF2, SNAI2, CYR61, MT-ND3, FLRT2, RGS3,
FXYD6, IGHG4, CADM1, RAI14, GADD45G, SMOC2, MT1E, CXCL12, IGHG1,
UNC5B, EMILIN1, TPST1, CDKN2A, APP, HAPLN1, GOLIM4, RAB31, NR3C1,
NNMT, EFNA5, LIMCH1, LPAR1, S100A4, JCHAIN, KCNQ1OT1, SESN3, PLD3,
IRX3, LAYN, ZEB1, WIPF1, CCDC3, HES1, HERPUD1, PSAP, IGFBP4, CCNG2,
CD9, POSTN, MMP11, APOL2, FCGRT, MT1X, CITED2, CKAP4, FNDC3B,
GGT5, C1orf54, FBLN7, ID2, CXCL14, CKB, ANKRD28, B4GALT1, MZB1,
YPEL2, MIR99AHG, PALLD, CTSZ, MAP4K4, PTPRS, C10orf10, SSR4,
S100A10, PDPN, CSF1, PPA1, BNIP3, LAMB1, CPE, RCAN1, NPC2, TIMP3,
CD276, QKI, and SRP14.
Cluster 1: PLAC9, NR4A1, CFH, MGP, CLU, CFD, LTBP4, MEG3, KLF4, CRLF1,
CES1, FOSB, C1R, AKAP12, GSN, S100A6, NR4A2, SQSTM1, C1S, MGST1,
SOD2, BTG2, JUND, LEPR, ADM, EFEMP1, GPRC5A, C3, LMNA, TWIST2, A2M,
KDM6B, TGFBR3, ATF3, STC1, APLP2, CEBPB, CREB5, PTGS2, HSPB8,
C11orf96, IGFBP6, KRT14, NR4A3, CXCL1, C8orf4, EGR1, RRAD, GSTP1,
SERPING1, FOSL1, DNAJB1, CXCL2, CEBPD, NEAT1, CELF2, PDLIM3, FABP3,
TXN, NAMPT, FST, GPC3, SERPINE2, HMOX1, DCN1, FBLN5, KRT6A, TRIB1,
DIO2, PNRC1, TNFAIP2, EIF4A3, TNFAIP6, H3F3B, KRT17, OSR2, UBC, IL6,
CD81, FTH1, APCDD1, MEDAG, FBLN2, FHL1, S100A2, PLPP3, FTL, RDH10,
NTRK2, AKR1C1, HSPB1, PERP, RHOB, DPYSL2, KRT5, CXCL8, EGR3, SRPX,
ZNF331, BHLHE40, WTAP, SFRP1, SOD3, ICAM1, TMEM176B, CHMP1B, IER3,
NFATC1, CDKN1A, TNFAIP3, NEFL, MAFF, CCDC80, DNAJA1, PIM1,
SERTAD1, NFKBIA, TGIF1, CD55, PBX1, VEGFA, BDKRB2, KRT75, IER2,
TIMP1, MT-CO3, MFAP5, WNT5A, LAMA2, HSP90AA1, NFIL3, DUSP1,
GADD45A, TNFSF13B, KRT6B, RAI2, NTM, CYBRD1, MAP3K8, STEAP2, EGR2,
IGF1, TPPP3, SELK, LITAF, VGLL3, MCL1, TNFRSF12A, STEAP1, REL,
PPP1R15A, PLTP, PIM3, KLF2, FXYD1, NCOA7, FILIP1L, GPX3, NAF1, ECM1,
GLA, FSTL1, CCL2, NFIB, GREM1, SLC3A2, LRP1, CRYAB, PRNP, TSHZ2,
PHLDA2, CCND2, PHLDB1, SOX9, NFIX, KPNA2, RND3, NFKB1, SERPINF11,
NFKBIZ, GYPC, LTBP2, GEM, MALAT1, GADD45B, CCDC71L, SLC2A3, PLPP1,
NTN4, THBS1, ABL2, CD63, CTD-3252C9.4, TMEM176A, RP11-553L6.5, RGS2,
DAB2, HSPA1A, DST, CRIP1, PRRX2, RSRP1, BASP1, NINJ1, IGFBP5,
HIST1H4C, FAM20C, HSPA1B, RGCC, MYADM, PTGES, NUPR1, SPTBN1,
PAPPA, F2R, TANK, UGP2, TP53INP2, WIPI1, TUBB2A, ARID5A, UBE2S,
ARID5B, NUCB2, ZFP36, PROS1, MME, EIF4E, DDAH2, SLC25A37, TWIST1,
AKR1B1, SEMA3B, FOSL2, XBP1, RHOBTB3, FOS, RORA, NFATC2, ATP2A2,
PHLDA1, GLRX, IDS, FBN1, PHACTR2, CREG1, FAM46A, ID1, LIMCH11,
FAM215B, PTMA, SEMA3C, TCF4, GLT8D21, LGALS3, FNDC4, OSBPL8,
PEA15, CAV1, UGCG, KRT10, TOB1, GLIS3, EMP1, and EMP3.
Cluster 2: GJA4, TINAGL1, COX4I2, ARHGAP15, PTP4A3, PPP1R14A, ADGRF5,
RGS5, MCAM, CPM, CCDC102B, NDUFA4L2, COL18A1, IGFBP7, COL4A1,
SPARCL1, MYL9, ACTA2, CALD1, ADIRF, NOTCH3, EPS8, CDH6, COL4A2,
NR2F2, PTMA1, SEP4, KCNJ8, PGF, LHFP, ESAM, EPAS1, ARHGDIB, TAGLN,
PARM1, MYLK, STEAP4, ANGPT2, APOLD1, MAP3K7CL, PLXDC1, EFHD1,
BCAM, PHLDA11, GJC1, PEAR1, MAP1B, TPM2, HOPX, FAM13C, MFGE8,
ITGA1, SOX13, ID4, ABCC9, RCAN2, ARHGAP29, LYPD1, GPR4, OAZ2,
EDNRB, RASGRP2, NFASC, C1QTNF1, ADAMTS4, GUCY1B3, HES5,
NEURL1B, CAV11, KRT18, SERPINI1, TPPP31, WFDC1, NDRG2, SYNPO2,
THY1, OLFML2A, ISYNA1, RCSD1, RBPMS, SOD31, TBX2, MAP2, CD59,
SEPHS2, PPP1R12B, IGFBP2, PDGFRB, JAG1, SCIN, CRIP2, RP3-395M20.12,
EGFLAM, ITGB1, CBFA2T3, CSRP2, MEF2C, CD36, CHCHD10, ADAMTS9,
ENPEP, ADAMTS1, LINC00152, HES4, GUCY1A3, TPM1, GUCY1A2, PDLIM1,
PDGFA, TFPI, CYGB, DSTN, EBF1, TSPAN12, MOCS1, KCNE4, FILIP1L1,
CRIP11, EGFL6, GPX31, FJX1, TNFRSF21, CHN1, HEY2, RPS6KA5, RERG,
PTGIR, SYTL2, IFITM2, LPP, B2M, TGFB1I1, RGS4, CRISPLD2, ADGRL2,
TRNP1, ASAH1, SENP3, PRKCDBP, RAMP1, CSPG4, C2CD2, LINC00839,
PROSER3, LURAP1L, FAM213A, BATF3, SMOC21, UACA, LBH, LRRC32, NES,
CEBPD1, LRRC8A, PDK4, HLA-A, MIR4435-2HG, PAG1, COL14A1, IFITM3,
ENTPD1, MALAT11, ACVRL1, ARHGAP24, CKM, COL5A3, CDKL5, DNAJC27,
HLA-E, TLE1, GEM1, PROCR, TNS2, PBLD, SRGAP1, SDSL, SEMA5A, NID1,
DLC1, CREB3L2, ID3, GMFG, LAMC1, HLA-F, BST2, PVRL2, EDNRA, PDLIM31,
NEXN, LINC00926, USP13, PTPRG, LGALS3BP, ZEB2, CACNB1, SOCS3,
COX7A1, TNS3, CNN3, CAMK1, CYP26B1, HSPB2, MX2, RAB13, SPRY1,
ANXA6, ADAM12, CDH13, RASD1, AXL, SYDE1, GBP2, NRP1, HMGN2, TOB11,
LAMA4, ARHGEF12, CD248, SLC2A31, IFI27, ISG15, ARL6IP1, YWHAH,
PHLDA3, TSPAN5, THOC2, TUBA1B, MARCKSL1, SFTA1P, PKIG, HLX, CTA-
29F11.1, ASAP2, CDKN1A1, MYC, FKBP5, IFIT3, PHYHD1, ARID5A1, NUDT4,
SMIM10, DLX51, SEMA4C, ZFHX3, TMEM204, ANKRD281, CCND1, DKK3,
HIF1A, PTRF, PLEKHO1, H2AFZ, VAMP5, MAP1LC3A, FAM127C, GNG11,
NSRP1, FARP1, IFI44L, C4orf3, KMT2E, COX20, ANKRD12, FAM133B, ITGAV,
YPEL5, PLPP11, LGMN, GOLGB1, CTSL, CC2D2A, JUNB, ITM2C, H1F0, VASN,
DYNLRB1, SPTBN11, MAFF1, ANTXR2, C2, DDIT3, ESF1, IFI6, INTS6, LTBR,
RHOB1, and ZC3H13.
Cluster 3: LGALS1, COL7A1, CTHRC11, SPON2, COL6A1, SCG5, LY6E,
COL1A11, COL6A2, FN1, NTM1, COL6A31, IL11, GREM11, ISG151, IFI61,
TPM21, COL3A11, SELM, HTRA3, WNT5A1, EMILIN11, COL5A11, INHBA,
TNFRSF12A1, PFN1, TMEM45A, SULF1, VCAN1, ITGA5, LOXL11, TMSB10,
MMP1, ADAMTS2, TGFBI, GLIPR2, S100A11, POSTN1, TNFAIP61, TWIST21,
COL5A21, ADAM19, SUGCT, LINC001521, COL5A31, SERPINH11, EVA1A,
MFAP51, STEAP11, MFAP21, IL24, ACTB, ISLR, CRABP2, GLIS31, IL61, BST21,
MMP141, BPGM, ADAM121, TNC, MT-CO2, F2R1, NREP, IFI271, LOXL2,
PTGES1, CCL21, NOX4, PTK7, HLA-B, TGM2, TPM11, COL15A1, HLA-C, TYMP,
IL32, MME1, IFITM1, PDLIM4, GBP1, SAT1, STC2, FAM19A5, BASP11, CALD11,
MMP111, RARRES2, PLAT, SERPINE1, HILPDA, C12orf75, WARS, PAPPA1,
S100A61, TAGLN1, CD82, WDFY1, SERINC2, KLF6, TMEM158, PRRX21, PLAU,
DIXDC1, AQP1, GCNT1, CXCL81, CHPF2, NES1, PXDN, S100A16, CD2481,
IFI44L1, CXCL3, MX21, XAF1, LAMA41, FSTL11, TRIOBP, CXCL11, GPM6B,
TIMP2, FAM129B, GUCY1A31, MAGEH1, PKM, PLXNC1, FTH11, HLA-A1,
GAPDH, HES41, WIPI11, IGFBP3, TUBA1A, PHLDA31, ANXA5, SCX, RP11-
115D19.1, GOLM11, IFIT1, OAS1, IFIT31, FKBP11, SGK1, ANPEP, MX1,
HIF1A1, BMP2, ENO1, TCTN3, CEP57L1, ITGAV1, MEST, SEC23A, METRNL,
ARL4C, F3, PMEPA1, H1F01, ANXA61, RORA1, ZFAND2A, APOL1, ANXA2,
AKR1B11, ALDH2, HSPA5, EIF5A, FARP11, LGMN1, IRF7, CHMP1B1, TUBA1C,
GNAI2, CREB3L21, ZMPSTE24, RP3-325F22.5, RIN2, ARID5A2, HSPH1,
HSPA1B1, and DNAJA11.
Cluster 4: S100A21, KRT141, BIK, KRT171, KRT6A1, ADRB2, LY6K, RPL35,
KRT19, KRT51, KRT31, PLPP2, PPP1R14B, KLK8, RAB38, SFN, BARX2, CTD-
2357A8.3, C19orf33, SDR16C5, SLCO4A1, ITGB6, KLC3, TNS4, TNNT1, P3H2,
C11orf45, SLC2A1, FXYD3, ATP1B1, DDR1, NDRG1, E2F7, SPINT1, IRF6,
LAD1, NRG1, APOC1, TBX1, PROM2, NELL2, FERMT1, POPDC3, GYLTL1B,
CXADR, ITGB4, FLRT3, TP73, TSGA10, CKMT1B, TXNDC17, GJB2, CLCA2,
RP11-54H7.4, PITX1, SOX15, PKP3, ST14, COL17A1, SERINC21, GBP6,
JSRP1, LAMC2, KRT15, CDH1, GSTP11, ITGA6, SERPINB5, ENO11, LYPD3,
GPR143, LAMB3, PERP1, KRT6B1, TRIM29, GAPDH1, CPA4, OCIAD2,
FAM84B, SPINT2, DSG3, TP63, NPW, ACOT7, DHCR24, FABP5, GJB6,
CALML3, JUP, PYGB, B3GNT3, NSG1, ANXA3, KRT8, LAMA3, CENPM, PVRL1,
EPCAM, AHNAK2, CDH3, CDT1, RBP1, ZWINT, TSTD1, SOX91, HCAR2, EHF,
EFNB1, KLF5, RPS29, HMGA1, PTHLH, S100A111, NRCAM, MYBL2, ZBED2,
MAD2L1, CKAP5, DSC3, APOBEC3D, ZNF490, EIF4EBP1, CENPW, S100A161,
DES, TUBA4A, SEPSECS, BEX2, ELF3, SCD, KIF21A, CXCL13, FGFBP1,
S100A14, EGLN3, ARL4D, CA9, ITGA2, EFNA1, DSG2, CDCA5, KANK1, KRT16,
FAM83A, MFSD8, C16orf59, IFI272, AC006262.5, TIMP11, GPC31, FSCN11,
CLDN7, RPLP1, NEFL1, CD109, LDHA, IFI62, CDK6, FABP4, CHI3L1, ODC1,
PCDH7, ADM1, SLC38A1, CXCL31, PKM1, ALDH3A1, PRSS8, KRT6C, CKS1B,
RP11-115D19.11, LGALS7B, CDC20, CA2, FAM216A, SNHG25, EZR, ZCWPW1,
SLC7A8, PRPF6, ERO1A, APOE, BMP21, LY6D, KIAA0753, CSTA, CYB5D1,
S100B, NDFIP2, XXbac-B135H6.18, KIF20B, SOX6, PRNP1, ASUN, UPP1,
PRKDC, HIST1H1C, MEST1, PNP, OVOL1, ATP1B3, IRX31, DAAM1, TUBB2A1,
RRAD1, SYNGR2, ZNF436, CTNND1, PCNA, PABPC1, TMSB101, TUBA1A1,
PTPN14, RP11-398K22.12, FECH, CD44, TFRC, ANKRD50, GLTP, MAGEH11,
CYB561D1, SFTA1P1, C1orf56, CDKN1C, TUBB2B, RHOBTB31, HMGB1,
PBXIP1, and SAT11.

### Fibroblast Phenotyping Reveals Novel Classification Paradigm in HNSCC

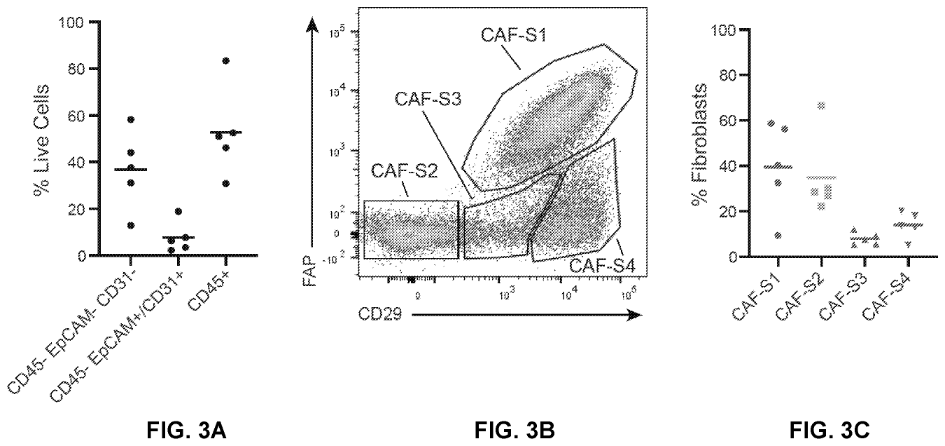

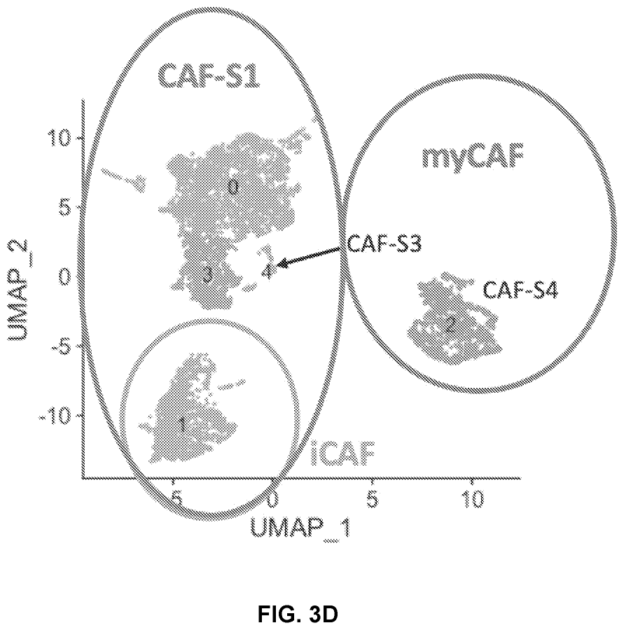

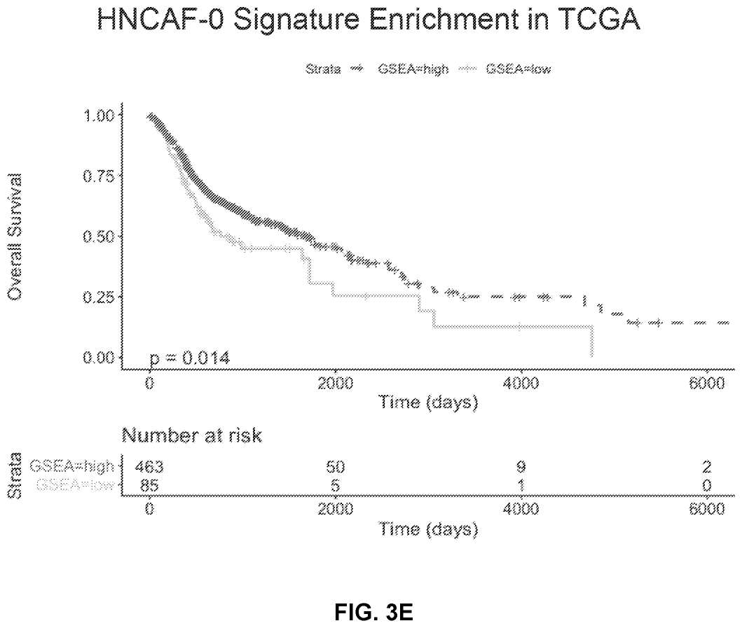

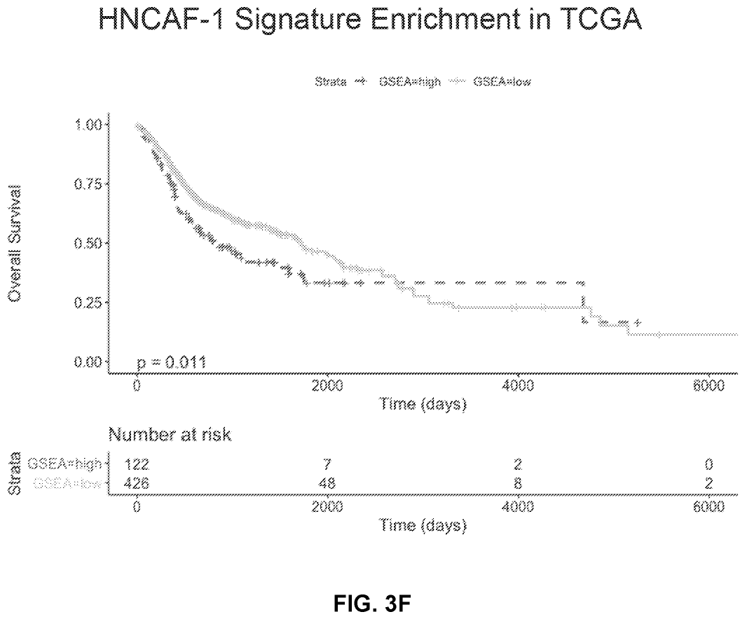

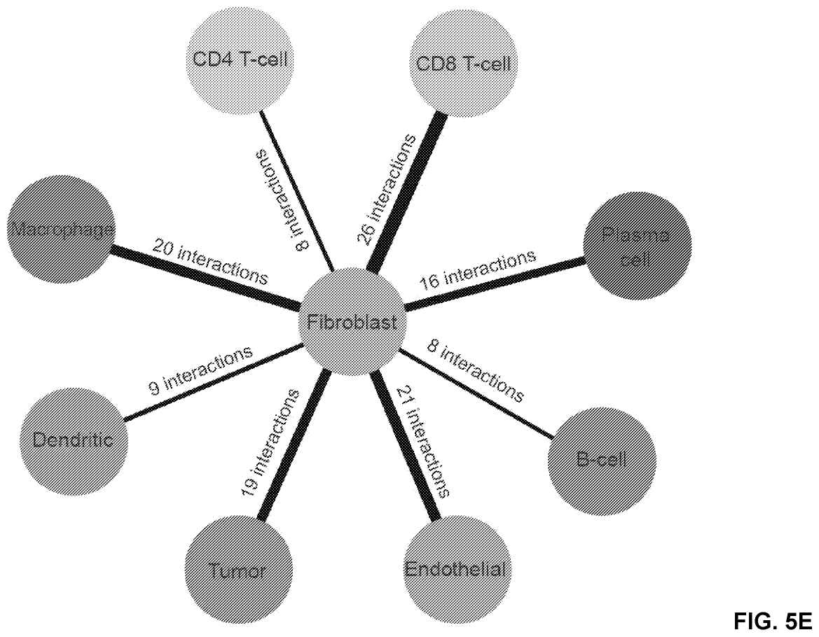

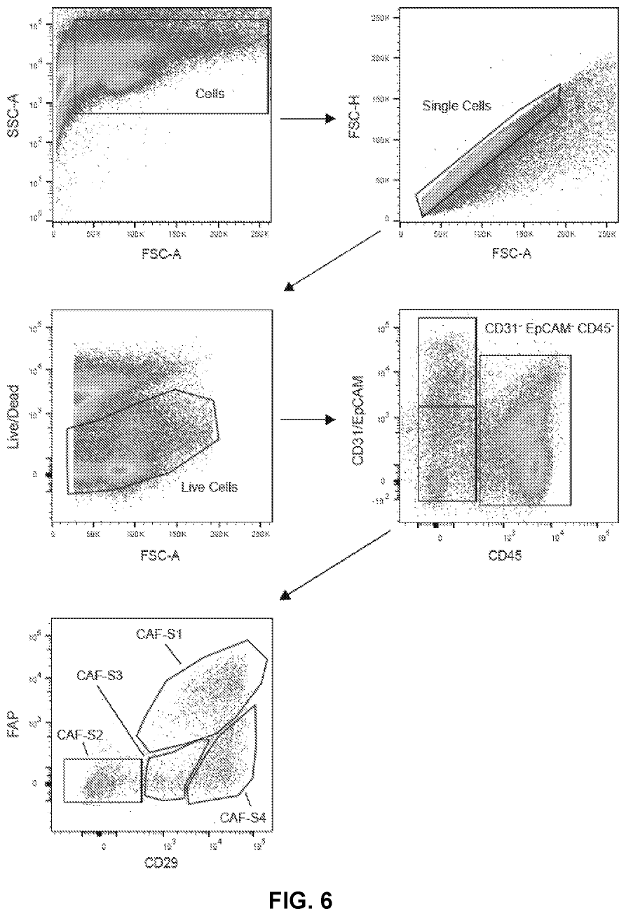

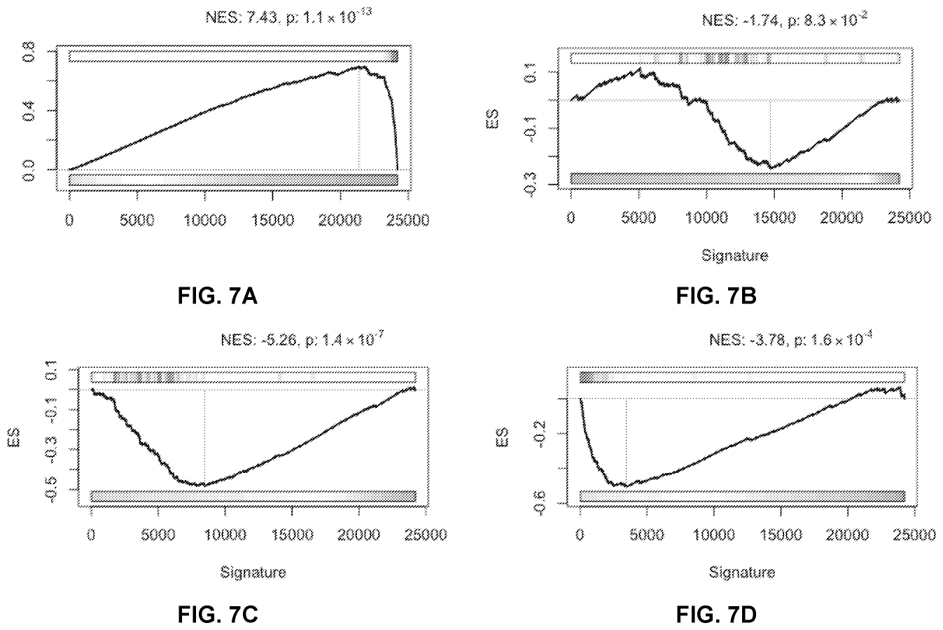

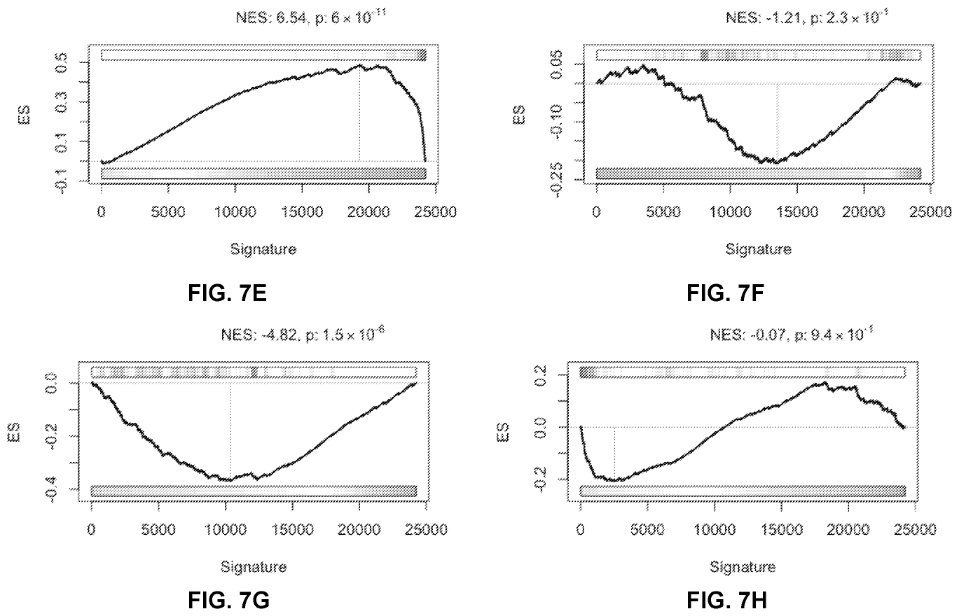

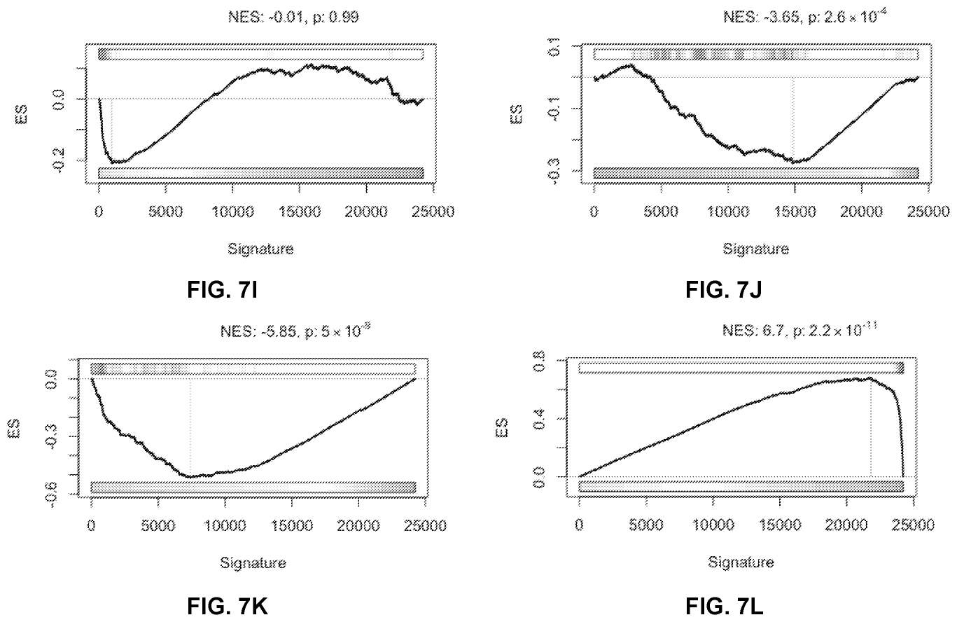

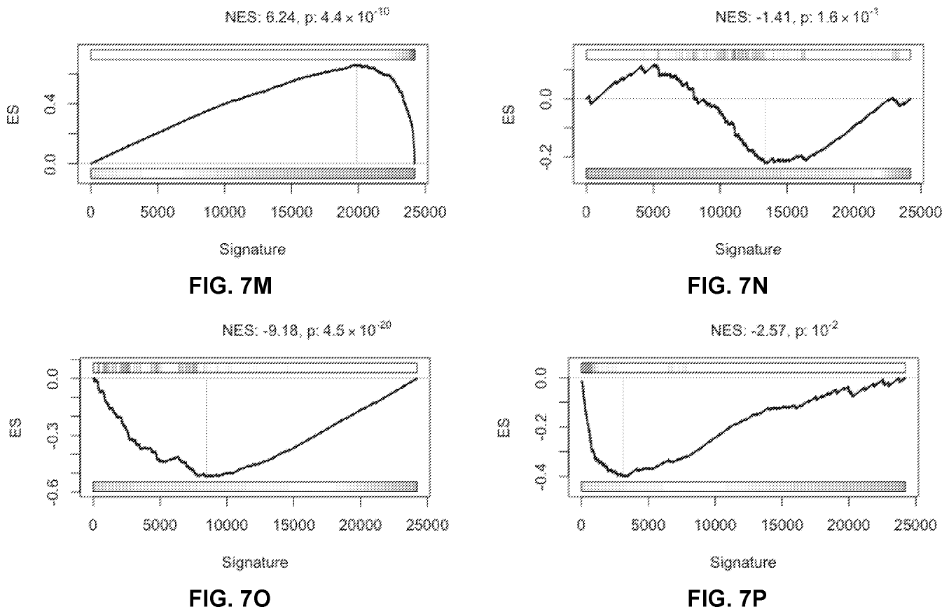

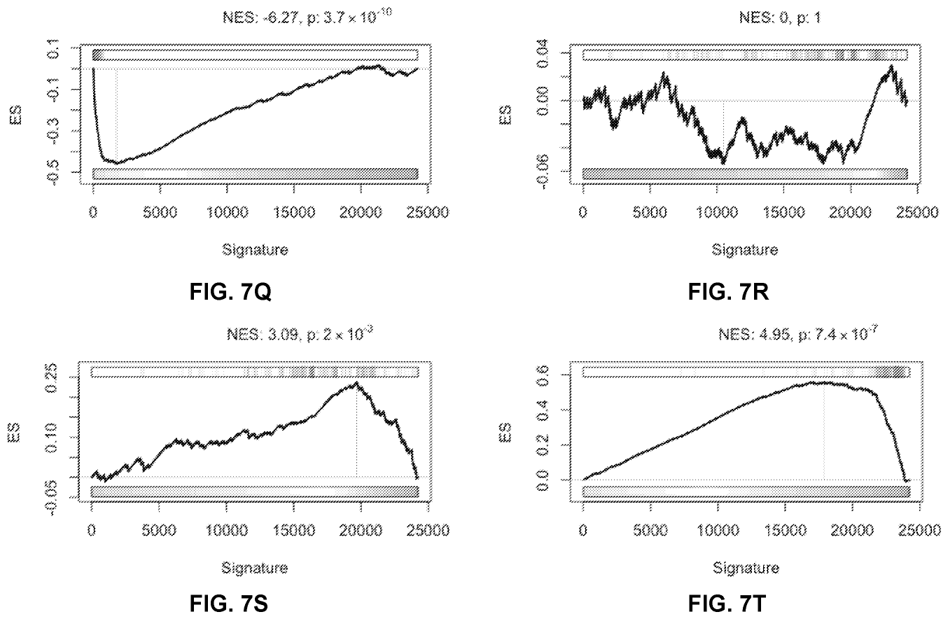

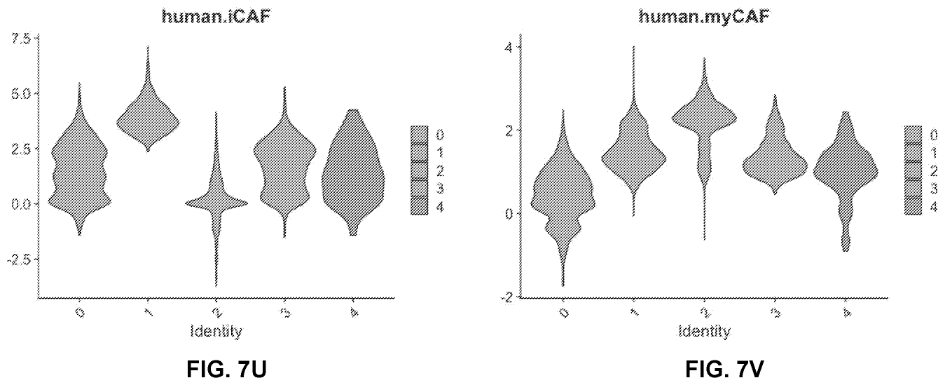

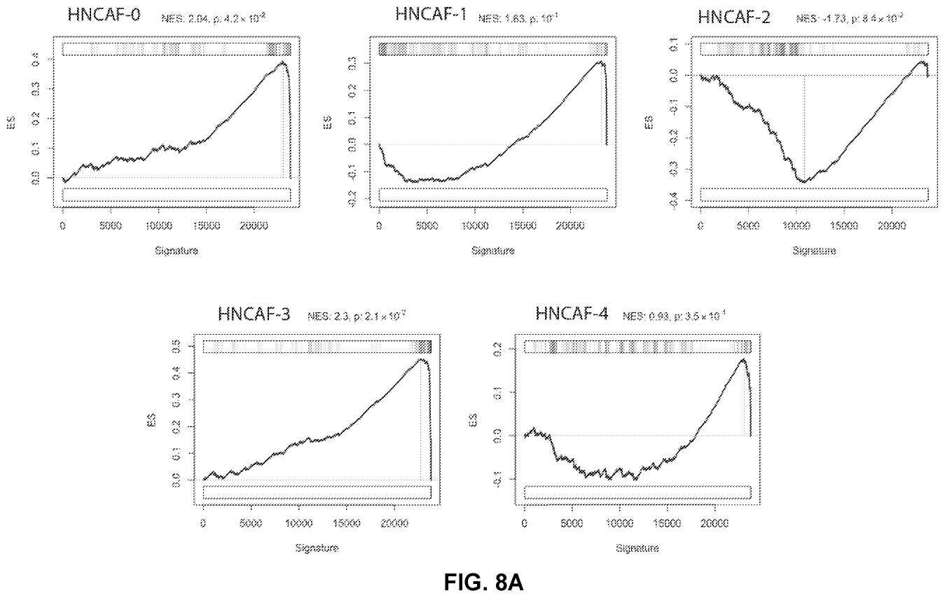

Next evaluated was the extent of CAF infiltration in HNSCC tumors by flow cytometry. CAF abundance—as defined by CD45⁻ EpCAM⁻ CD31⁻—ranged between 12% and 58% of the total live cells (FIG. 3A). Having confirmed the presence of CAF in human head and neck tumors, the presence of novel HNCAF subpopulations predicted by the analysis was assessed. Distinct CAF subpopulations termed CAF-S1, CAF-S2, CAF-S3, and CAF-S4 have been previously identified in breast cancer based on the expression of CD29 and fibroblast activation protein (FAP) by flow cytometry. Thus, before correlating these CAFs to the five HNCAFs identified by our analysis, first determined was whether these CAFs, as defined by specific FACS markers, were represented in HNSCC tumors. Following the same gating strategy employed by Costa et al. (FIG. 6), the presence of all four breast cancer CAF populations in HNSCC (FIG. 3B) was confirmed. Interestingly, CAF-S1 and CAF-S2 were most abundant, while CAF-S3 and CAF-S4 abundance was quite minimal (FIG. 3C). Given the presence of all four populations, whether the HNCAFs identified from single-cell RNA-Seq aligned with the breast cancer CAF groups was assessed. Specifically, CAF-S1-S4 cells from HNSCC tumors were first sorted according to the gating strategy in FIG. 6, and then bulk RNA sequencing of each subpopulation was performed. Pairwise gene set enrichment analysis of the HNCAF protein activity signatures in the bulk transcriptome indicated that the gene sets representative of HNCAF-0, HNCAF-1, and HNCAF-3 were all enriched in the same breast subtype (CAF-S1), while HNCAF-2 and HNCAF-4 were also enriched in CAF-S4 and CAF-S3, respectively (FIGS. 7A-7T) and CAF-S2—primarily defined as double-negative for FAP and CD29—did not significantly align with any HNCAF. Crucially, the clustering solution generated by protein activity analysis of single cells provided much greater resolution and functional characterization compared to the CAF-S1/S2/S3/S4 phenotyping paradigm. Specifically, the CAF-S1 subtype matched three distinct HNFAC subtypes, which have opposite association with clinical responses to immunotherapy.

Concordance of the classification schema with previously defined gene set markers of inflammatory CAFs (iCAFs) and myofibroblastic CAFs (myCAFs), as first described in pancreatic cancer, was tested. Cell-by-cell enrichment of iCAF and myCAF gene signatures revealed an enrichment for the iCAF signature in HNCAF-1 cells and for myCAFs in HNCAF-2 cells (FIG. 7U-7V). Correlations between the HNCAF populations and the breast or pancreatic CAF phenotypes is summarized in the classification scheme shown in FIG. 3D. in contrast, while presenting some similarity to CAF-S1 cells, it was conclude that HNCAF-0 and HNCAF-3 represent novel, molecularly distinct fibroblast subpopulations, unique to head and neck cancer and predictive of patient outcome, which do not match either the iCAF or myCAF phenotype. Bulk RNA-Seq analysis of multiple normal fibroblasts from upper digestive tracts cultured in vitro, failed to identify a match with HNCAF cells. Cumulatively, these data demonstrate the complex heterogeneity, potential plasticity, and outcome predictive value of CAF cells in HNSCC and between tumor types, and highlight the distinctions between HNCAFs and previously identified CAF subpopulations.

### HNCAF-0 Predicts Favorable Disease Course in TCGA, in Contrast to HNCAF-1.

To evaluate the prognostic relevance of the CAF populations identified by the analysis also in a setting free of external immunotherapeutic pressures, enrichment of HNCAF-0 and HNCAF-1 protein activity signatures in The Cancer Genome Atlas (TCGA) HNSCC cohort was measured. Gene set enrichment (GSEA) analysis, on a patient-by-patient basis, revealed significant enrichment of the HNCAF-0 signature in patients with better overall survival in TCGA (FIG. 3E), suggests that these cells may not only be important regulators of immunotherapy response but may also plays a key role in mounting endogenous immune responses against HNSCC cells. In contrast, the HNCAF-1 protein activity signature was associated with worse overall survival suggesting a potentially immunosuppressive role (FIG. 3F). These data highlight the importance of differentiating the functional role of distinct CAF cells. Indeed, despite the fact that they both show similarity to CAF-S1 cells, as identified by prior studies, they predict opposing disease courses and have likely opposing functions in the TME.

### HNCAF-0 Cells Inhibit TGFβ Dependent T-Cell Exhaustion in Functional Co-Culture Experiments.

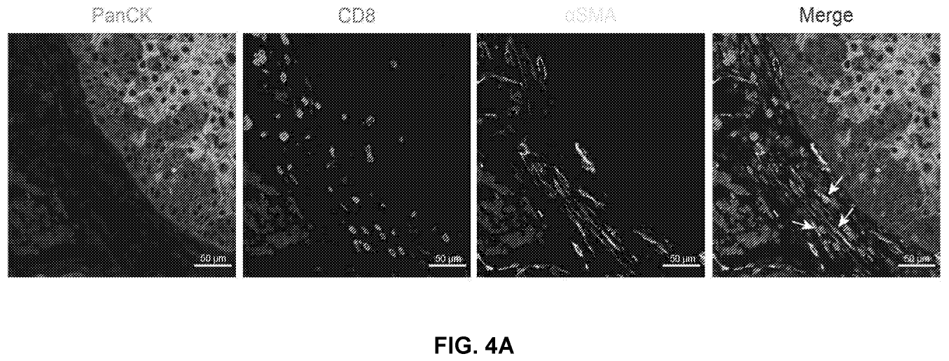

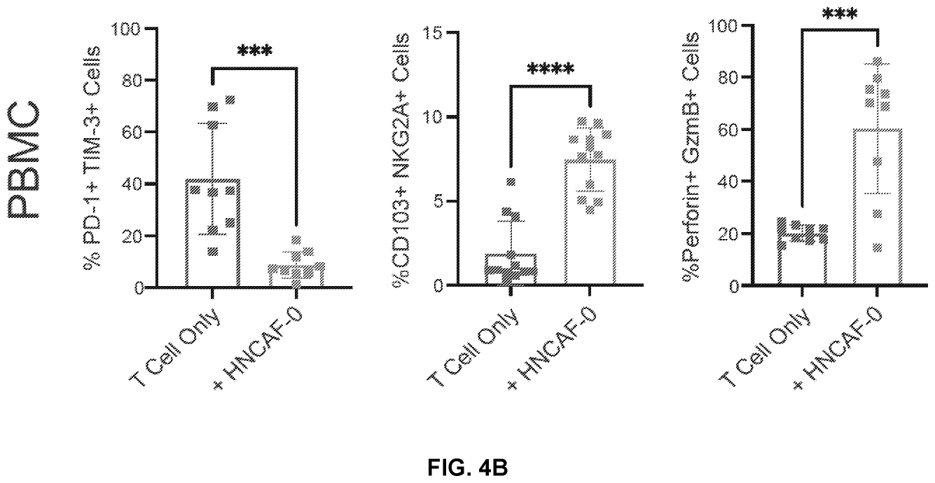

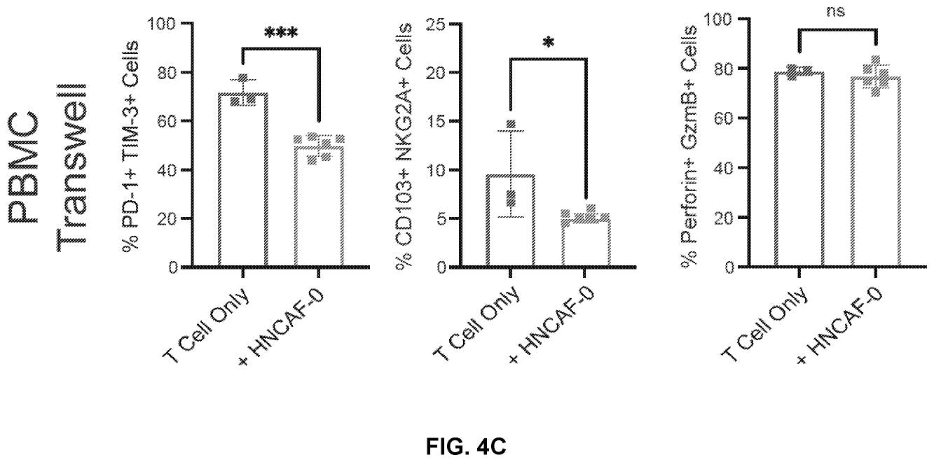

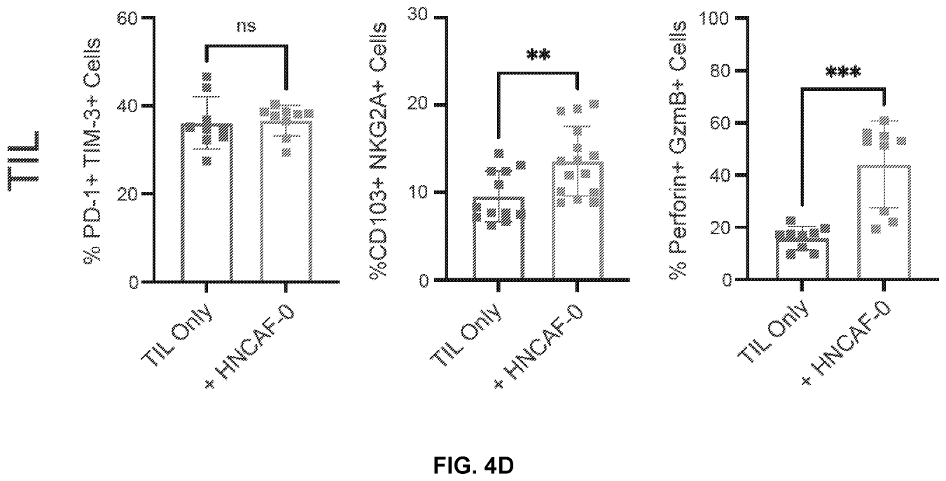

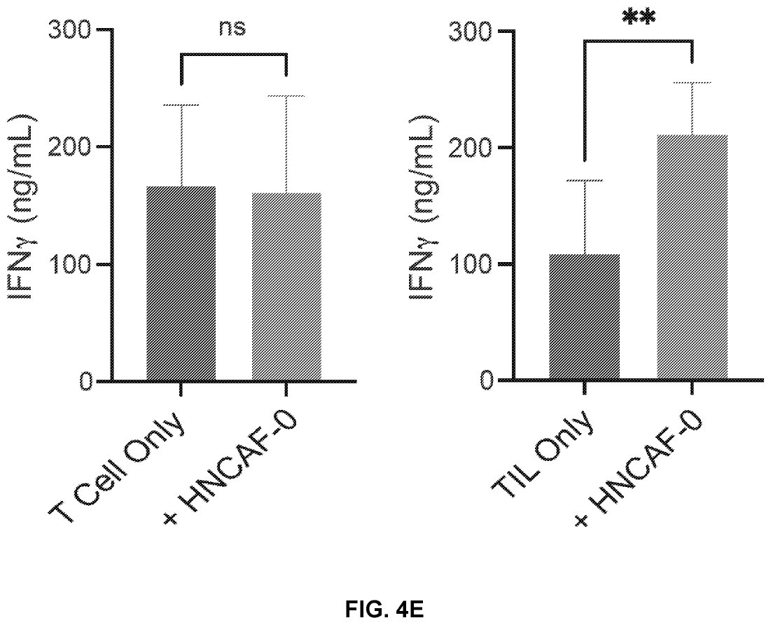

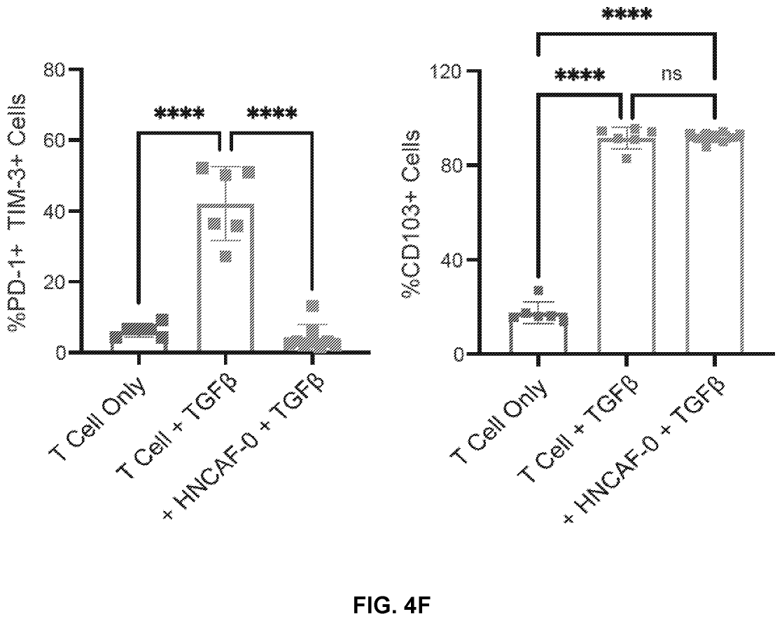

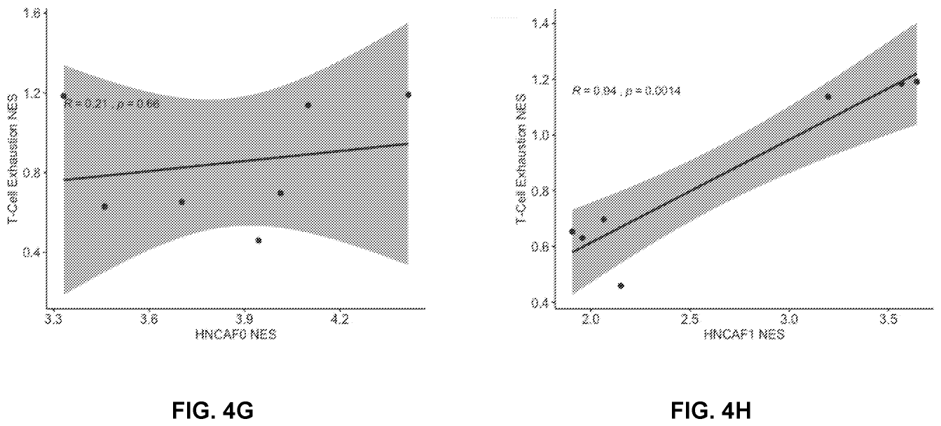

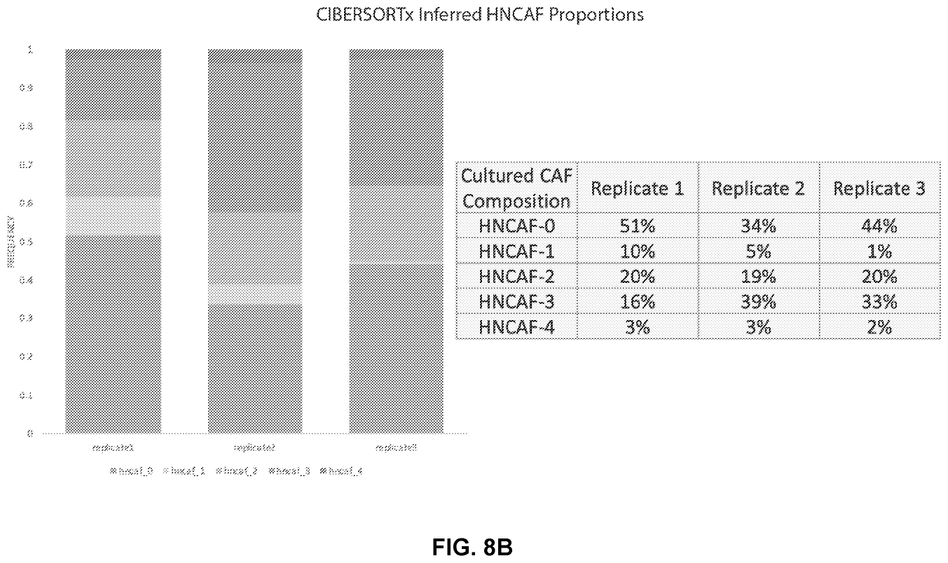

Prompted by these intriguing clinical findings (FIG. 2D), the potential interactions of HNCAF-0 cells with other TME subpopulations was studied. Interactome analysis showed that HNCAFs have more receptor-ligand interactions with CD8 T-cells than with any other cell subtype in the TME (FIG. 5E). Therefore, it was decided to further investigate the relationship between HNCAF-0 and human CD8 T cells in situ and in vitro. Digital spatial profiling (DSP, NanoString) of immune-related transcripts and protein markers was performed on HNSCC tissue from patients prior to nivolumab treatment. Multiplexed immunofluorescent images exhibited colocalization of CAF with CD8+ T cells in the stromal compartment (FIG. 4A). To test whether HNCAF-0 cells may directly affect the biology of the T cell repertoire, in vitro co-culture assays were performed with HNCAF harvested from surgical resection and either naïve T cells or tumor-infiltrating T cells. Primary HNCAF-0 fibroblasts were isolated from human HNSCC samples and their transcriptional identity was verified by RNA-Sequencing and protein activity analysis (FIG. 8). When co-cultured with CD3+ T cells isolated from peripheral blood mononuclear cells of healthy donors, HNCAF-0 cells reduced the PD-1+TIM-3+ exhaustion phenotype among exogenously activated T cells and increased the CD103+NKG2A+ tissue resident memory phenotype, as well as cytotoxicity, based on Perforin and Granzyme B assays (FIG. 4B). Transwell co-culture assays revealed that by HNCAF-0-mediated T-cell activation increase and induction of tissue resident memory phenotypes—but not T-cell exhaustion phenotype mitigation-depends on cell-to-cell contact (FIG. 4C). Additionally, coculture of HNCAF-0 cells with CD3+ tumor-infiltrating T cells isolated directly from human HNSCC resulted in an equivalent increase in tissue resident memory cells and cytotoxicity; in contrast, however, HNCAF-0 cells could not rescue the exhaustion phenotype of terminally exhausted tumor-infiltrating T cells (FIG. 4D). Interestingly, HNCAF-0 cells strongly promoted production of the activation markers, perforin, granzyme B, and IFNγ, in tumor-infiltrating T cells (FIG. 4D-4E), suggesting that while these cells may prevent exhaustion in early activated T cells, they may not be able to reverse the phenotype in already exhausted TME cells. In addition, HNCAF-0 cells also increased cytolytic activity and function of non-exhausted T cells in the TME. Notably, it was found that HNCAF-0 completely rescued by TGFβ-mediated PD-1/TIM-3 induction in culture, without inhibiting total TGFβ signaling (as defined by CD103 induction), suggesting HNCAF-0 cells may prevent exhaustion through a mechanism specific to these checkpoint receptors (FIG. 4G).

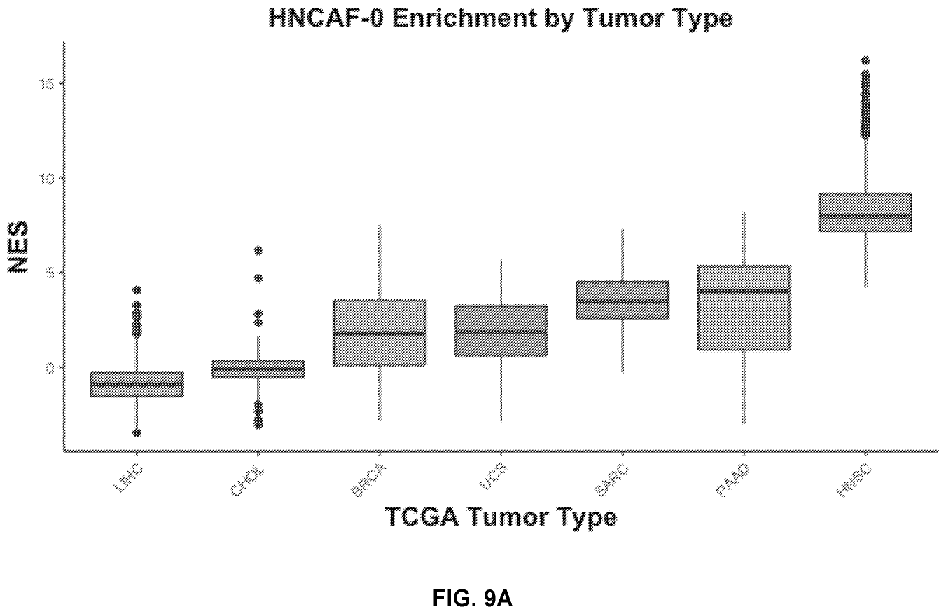

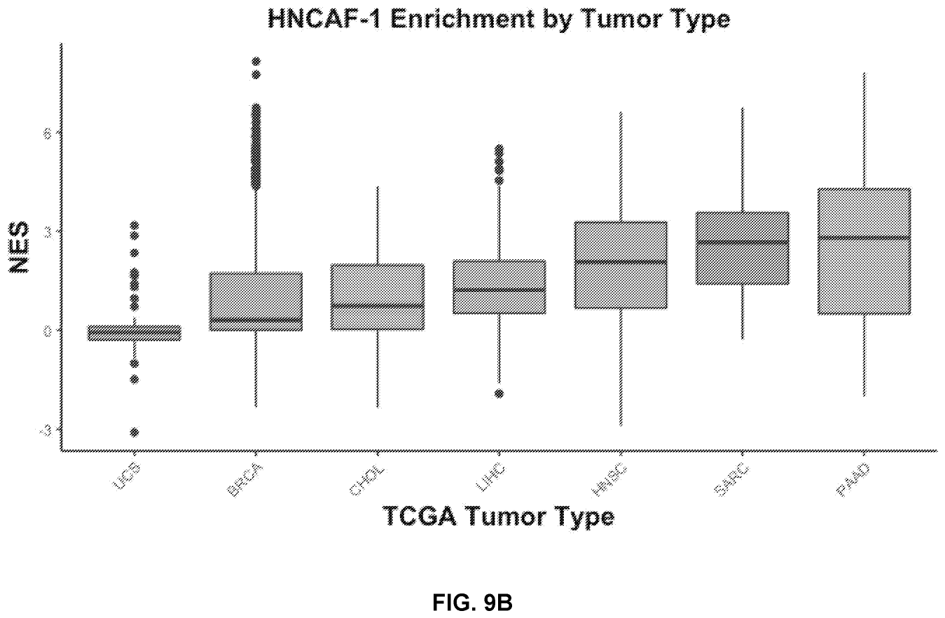

To evaluate CAF influences on T cell exhaustion in situ, the digital spatial profiling data was further leveraged to evaluate colocalization of HNCAF-0 and HNCAF-1 protein activity signatures in regions enriched for the T-cell functional exhaustion signature. Indeed, the HNCAF-1 signature enrichment significantly correlated with increased T cell exhaustion signature enrichment (r=0.94, p=0.0014). In sharp contrast, the HNCAF-0 signature was not significantly associated with a T-cell exhaustion signature in the TME region of interest (FIG. 4G-4H). Given the association of HNCAF-1 cells with an immunosuppressive environment, it was aimed to evaluate the direct impacts of sorted HNCAF-1 cells on T cell phenotypes in co-culture, as performed for HNCAF-0 cells. However, despite repeated experiments, T cells co-cultured with HNCAF-1 rapidly died, leaving an insufficient number of viable cells for flow analyses (data not shown). T cell death induction was not observed when T cells were cultured in isolation or with HNCAF-0 cells, suggesting HNCAF-1-mediated acceleration T cell apoptosis. Additionally, enrichment of HNCAF protein activity signatures across TCGA, by tumor type, focusing on tumors with high stromal cell content, revealed that HNCAF-0 enrichment is relatively specific to HNSCC while HNCAF-1 enrichment is more broadly observed (FIG. 9A-9B). Intriguingly, HNCAF-1 enrichment is highest in pancreatic adenocarcinoma, which is known to be unresponsive to PD-1 based immunotherapy (FIG. 9B), where it phenotypically matches the previously defined iCAF population (FIG. 3D).

### Methods

### Clinical Design and Tissue Collection

Biospecimens were harvested from a window of opportunity trial of locally advanced HNSCC patients (oral cavity, oropharynx, larynx, hypopharynx) who were candidates for primary surgical intervention with curative intent (NCT03238365). All enrolled patients were treated with 1 month of 240 mg nivolumab every 2 weeks for 2 doses prior to definitive surgery (N=50). Half of the patients received tadalafil, and no differences were noted in response rates between the two cohorts. Consented patients were required to have fresh pre-nivolumab biopsy as well pre and post imaging. Meta-clinical annotation using pathological criteria was used to delineate paired subject specimens as responders vs. non-responders. For both pre and post treatment timepoints, fresh specimens were collected for frozen fixation, paraffin embedded fixation, and processed for both bulk and single cell transcriptomic sequencing.

### Tissue Dissociation

Fresh head and neck squamous cell carcinoma tumor specimens were collected in DMEM supplemented with streptomycin (200 mg/ml), penicillin (200 U/ml), and amphotericin B (250 mg/mL). Tumor specimens were dipped in 70% ethanol, minced to 2-4 mm sized pieces in separate 6-cm dishes, and digested to single cell suspension using the Miltenyi Biotec human tumor dissociation kit (Miltenyi Biotec #130-095-929) on the Miltenyi gentleMACS Octo dissociator (Miltenyi Biotec #130-096-427) following manufacturer's instructions. Dissociated cells were aliquoted for single-cell sequencing, flow cytometry analysis, or CAF culture.

### Single-Cell RNA-Sequencing

Samples were processed to generate single-cell gene expression profiles (scRNA-Seq) using the 10× Chromium 3′ Library and Gel Bead Kit (10× Genomics), following the manufacturer's user guide. After GelBead in-Emulsion reverse transcription (GEM-RT) reaction, 12-15 cycles of polymerase chain reaction (PCR) amplification were performed to obtain cDNAs used for RNAseq library generation. Libraries were prepared following the manufacturer's user guide and sequenced on the Illumina NovaSeq 6000 Sequencing System. Gene expression data were processed with “cellranger count” on the pre-built human reference set of 30,727 genes to generate counts matrices. Cell Ranger performed default filtering for quality control, and produced for each sample a barcodes.tsv, genes.tsv, and matrix.mts file containing counts of transcripts for each sample, such that the expression of each gene is in terms of the number of unique molecular identifiers (UMIs) tagged to cDNA molecules corresponding to that gene. These data were loaded into the R version 3.6.1 programming environment, where the publicly available Seurat package v3.0 was used to further quality-control filter cells to those with fewer than 10% mitochondrial RNA content, more than 1,500 unique UMI counts, and fewer than 15,000 unique UMI counts.

### Single-Cell Data Processing

Gene Expression UMI count matrices for each sample were normalized and scaled in R using the Seurat SCTransform command to perform a regularized negative binomial regression based on the 3000 most variable genes. Scaled data from each patient were batch-corrected by Seurat using the functions FindIntegrationAnchors and IntegrateData, with default parameters. The resulting dataset included 22906 high-quality cells (mean UMI count 4802) across four patients, including both pre-treatment and post-treatment time points for each patient (Patient1: 5857 pre-treatment, 7360 post-treatment, Patient2: 4938 pre-treatment, 1550 post-treatment, Patient3: 487 pre-treatment, 1741 post-treatment, Patient4: 401 pre-treatment, 572 post-treatment). The batch-corrected dataset was projected into its first 50 principal components using the RunPCA function in Seurat, and further reduced into a 2-dimensional visualization space using the RunUMAP function with method umap-learn and Pearson correlation as the distance metric between cells. The data were clustered by the Louvain algorithm with silhouette score resolution-optimization selecting the resolution with maximum bootstrapped silhouette score in the range of resolution from 0.01 to 1.0 incremented by 0.01. This resulted in an initial coarse clustering on gene expression (FIG. 5C). Within each cluster metaCells were computed for downstream regulatory network inference by summing SCTransform-corrected template counts for the 10 nearest neighbors of each cell by Pearson correlation distance.

For each single cell, inference of cell type was performed using the SingleR package and the preloaded Blueprint-ENCODE reference, which includes normalized gene expression values for 259 bulk RNASeq samples generated by Blueprint and ENCODE from 43 distinct cell types representing pure populations of stroma and immune cells. The SingleR algorithm computes correlation between each individual cell and each of the 259 reference samples, and then assigns both a label of the cell type with highest average correlation to the individual cell and a p-value computed by wilcox test of correlation to that cell type compared to all other cell types. Highest-Frequency SingleR labels within each cluster among labels with p<0.05 are projected into the Gene Expression UMAP space in FIG. 5D, such that localization of SingleR labels is highly concordant with the unsupervised clustering. Unsupervised Clusters determined by the resolution-optimized Louvain algorithm are therefore labelled as a particular cell type based on the most-represented SingleR cell type label within that cluster.

Differential gene expression analysis between single cell clusters throughout the manuscript is computed by the MAST hurdle model, as implemented in the Seurat FindAllMarkers command, with a log-fold change threshold of 0.5 and minimum fractional expression threshold of 0.25, indicating that the resulting gene markers for each cluster are restricted to those with log fold change greater than 0 and non-zero expression in at least 25% of the cells in the cluster.

### Regulatory Network Inference

From the integrated dataset, metaCells were computed within each gene expression-inferred cluster by summing SCTransform-corrected template counts for the 10 nearest neighbors of each cell by Pearson correlation distance. 200 metaCells per cluster were sampled to compute a regulatory network from each cluster. All regulatory networks were reverse engineered by the ARACNe algorithm. ARACNe was run with 100 bootstrap iterations using 1785 transcription factors (genes annotated in gene ontology molecular function database as GO:0003700, “transcription factor activity”, or as GO:0003677, “DNA binding” and GO:0030528, “transcription regulator activity”, or as GO:0003677 and GO:0045449, “regulation of transcription”), 668 transcriptional cofactors (a manually curated list, not overlapping with the transcription factor list, built upon genes annotated as GO:0003712, “transcription cofactor activity”, or GO:0030528 or GO:0045449), 3455 signaling pathway related genes (annotated in GO biological process database as GO:0007165, “signal transduction” and in GO cellular component database as GO:0005622, “intracellular” or GO:0005886, “plasma membrane”), and 3620 surface markers (annotated as GO:0005886 or as GO:0009986, “cell surface”). ARACNe is only run on these gene sets so as to limit protein activity inference to proteins with biologically meaningful downstream regulatory targets, and we do not apply ARACNe to infer regulatory networks for proteins with no known signaling or transcriptional activity for which protein activity may be difficult to biologically interpret. Parameters were set to zero DPI (Data Processing Inequality) tolerance and MI (Mutual Information) p-value threshold of 10⁻⁸, computed by permuting the original dataset as a null model. Each gene list used to run ARACNe is available on github.

### Protein Activity Inference

Protein activity was inferred for all cells by running the metaVIPER algorithm, using all cluster-specific ARACNe networks, on the SCTransform-scaled and Anchor-Integrated gene expression signature of single cells from each patient. Because the SCTransform Anchor-Integrated scaled gene expression signature is already normalized as an internal signature comparing all cells to the mean expression in the dataset, VIPER normalization parameter was set to “none.” The resulting VIPER matrix included 1239 proteins with activity successfully inferred from ARACNe gene regulatory networks. VIPER-Inferred Protein Activity matrix was loaded into a Seurat Object with CreateSeuratObject, then projected into its first 50 principal components using the RunPCA function in Seurat, and further reduced into a 2-dimensional visualization space using the RunUMAP function with method umap-learn and Pearson correlation as the distance metric between cells. Clustering was performed by resolution-optimized Louvain algorithm, as for gene expression (FIG. 1A), and SingleR-inferred cell type labels were carried over to identify cluster-by-cluster cell type labels (FIG. 1D). Differential Protein Activity between clusters identified by resolution-optimized Louvain was computed using bootstrapped t-test, run with 100 bootstraps, and top proteins for each cluster were ranked by p-value (FIG. 1C). The entire pipeline is implemented as in Obradovic et. al., 2021. Cluster cell counts were normalized to a fraction of the total sample separately for each patient and separately for pre-treatment and post-treatment samples, with differences in pre-treatment vs post-treatment frequency distribution plotted in FIG. 1B.

Association of HNCAF Signatures with Response to Immunotherapy

Fibroblast clusters including 5,414 cells from overall VIPER clustering of all cells were further isolated and sub-clustered (FIG. 2A), with differential protein activity and frequency pre-treatment vs post-treatment compared as in the analysis of initial clustering for all cells. A proteomic gene set for each head and neck cancer-associated fibroblast (HNCAF) cluster was defined based on proteins differentially upregulated in each cluster (see Table 2 for marker gene lists). In the dataset of clinical trial patients profiled by bulkRNASeq that had been annotated with subsequent response (n=9) or non-response (n=19) to αPD1 immunotherap, VIPER transformation was applied using the single-cell ARACNe networks on z-score scaled log 10(TPM) counts from pretreatment bulk-RNA-Seq data, and computed a differential protein activity signature ranking proteins by most upregulated in responders to most downregulated in responders. Enrichment of each HNCAF cluster marker set in the VIPER-transformed signature of responders vs nonresponders from bulkRNASeq was computed by Gene Set Enrichment Analysis (GSEA), with normalized enrichment score and p-value determined by 1000 random permutations of gene labels (FIG. 2D).

Clinical association of HNCAF cluster 0 and cluster 1 signatures with outcome was further tested in TCGA head and neck cancer cohort processed by ARACNe and VIPER as above. Sample-by-Sample Normalized Enrichment Scores were computed ranking VIPER-inferred protein activity in each patient sample from highest to lowest activity and then applying GSEA. Normalized Enrichment scores for HNCAF cluster signatures were binarized to less than zero (low) or greater than zero (high), and Kaplan-Meier curve showing association with Overall Survival time was plotted along with the log-rank p-value (FIG. 4A, 4B), such that HNCAF-0 enrichment is associated with improved overall survival (p=0.014, median survival time=602 days vs 1671 days) and HNCAF-1 enrichment is associated with worse overall survival (p=0.011, median survival time=1718 days vs 773 days). the sample-by-sample enrichment of these HNCAF populations among different TCGA tumor types with high stromal involvement (HNSC, PAAD, SARC, UCS, BRCA, CHOL, LIHC) was plotted and the distribution of these enrichment scores by tumor type was plotted to assess relative tumor-type specificity of the identified HNCAF signatures (FIG. 8).

### Digital Spatial Profiling

Nanostring GeoMX Digital Spatial profiling was further applied, profiling 10360 immune gene panel expression among three regions of interest (ROIs) from one patient and four ROIs from another. Anti-CD8, anti-αSMA, anti-PanCK, and DAPI stains were used for morphology identification and ROIs were selected based on high abundance of tumor (PanCK), cytolytic T cells (CD8), and fibroblasts (αSMA). ROIs were split into PanCK-positive and PanCK-negative components, with gene expression evaluated separately in each. In order to further assess spatial co-localization of HNCAF subtypes with functionally exhausted T-cells, segment-by-segment gene set enrichment of HNCAF cluster 0 and HNCAF cluster 1 markers as well as enrichment of a published T-cell exhaustion signature were applied, and normalized enrichment scores for these populations between spatial segments were correlated (FIG. 4G, 4H).

### CAF Phenotyping

In order to assess phenotypic concordance between prior fibroblast categorizations, including CAF-S1/S2/S3/S4 subtypes described by in the setting of breast cancer and iCAF/myCAF subtypes described by in the setting of pancreatic cancer, pairwise gene set enrichment of fibroblast phenotype marker gene sets among our HNCAF clusters identified by scRNA-Seq was performed. Published iCAF/myCAF gene sets were directly tested by GSEA for enrichment in each single-cell, with resulting enrichment scores plotted by HNCAF cell cluster in FIG. 6B, such that cells in HNCAF-1 are enriched for iCAF gene signature and cells in HNCAF-2 are enriched for myCAF signature. For CAF-S1/S2/S3/S4 phenotype-matching, S1/S2/S3/S4 cells were sorted by FACS using the gating strategy described by Costa et. al. (FIG. 6), and bulk-RNA Sequencing of each sorted population and differential gene expression of each population was subsequently performed against the mean to define population-specific gene expression signatures, with genes ranked from most differentially-upregulated to most differentially-downregulated in CAF-S1/S2/S3/S4. Pairwise Gene Set Enrichment Analysis of HNCAF cluster marker gene sets among CAF-S1/S2/S3/S4 gene signatures was then performed (FIG. 6A). Findings were highlighted that CAF-S1 gene signature was significantly enriched for the gene sets of HNCAF-0 (NES=7.43, p=1.1*10⁻¹³), HNCAF-1 (NES=6.54, p=6*10⁻¹¹), and HNCAF-3 (NES=6.24, p=4.4*10⁻¹⁰) CAF-S2 gene signature was not significantly enriched for any HNCAF population, CAF-S3 signature was significantly enriched for HNCAF-4 gene set (NES=3.09, p=2*10⁻³), and CAF-S4 signature was significantly enriched for HNCAF-2 gene set (NES=6.7, p=2.2*10⁻¹¹). This phenotypic classification scheme is shown on FIG. 3D and highlights the distinction between the HNCAF categorization observed from scRNA-Seq and prior CAF classification paradigms.

### Receptor-Ligand Interactions

Receptor-Ligand Interactions were inferred between coarse-grained cell types using 2,557 high-quality receptor-ligand interactions reported the RIKEN FANTOM5 database. This list of receptor-ligand pairs was filtered to identify pairs where the ligand was significantly upregulated, at the gene expression level, in at least one subpopulation, across patients, and the corresponding receptor was significantly activated in another subtype, based on VIPER protein activity analysis. These were further filtered to interactions to those detected in cancer-associated fibroblasts and plotted the number of unique receptor-ligand interaction pairs inferred between fibroblasts and each other detected subpopulation (FIG. 5E).

### CAF Isolation and Culture

Fresh head and neck squamous cell carcinoma tumor specimens were processed to single cell suspension as described above. For HNCAF-0/3, tumor single cell suspension was cultured in DMEM supplemented with 10% FBS, streptomycin (100 mg/ml), and penicillin (100 U/ml) for 2-3 weeks at 37° C. until fibroblasts grew out. For HNCAF-1, tumor single cell suspension was cultured in pericyte medium (ScienCell #1201) supplemented with 2% FBS, streptomycin (100 mg/ml), and penicillin (100 U/ml) for 2-3 weeks at 37° C. until fibroblasts grew out. To verify CAF identity, RNA was isolated from CAF lysates using TRIzol (Invitrogen #10296010) and sent for bulk RNA sequencing. Gene set enrichment analyses for the HNCAF subtype protein activity signatures were then performed on the bulk sequencing data, along with inference of cell type proportions by CIBERSORTx. Fibroblasts were passaged when cultures reached ˜80% confluence and all experiments were performed with CAFs under 10 passages.

### T Cell Isolation

CD3+T lymphocytes were isolated from the peripheral blood of healthy human donors. Peripheral blood mononuclear cells (PBMCs) were isolated using Ficoll-Paque Plus, following manufacturer's instructions. CD3+ T cells were isolated from PBMCs using magnetic bead sort with the MojoSort™ Human CD3 T Cell Isolation Kit (Biolegend #480022) according to manufacturer's instructions. For isolation of CD3+ tumor-infiltrating lymphocytes (TILs), fresh head and neck squamous cell carcinoma tumor specimens were processed to single cell suspension as described above. CD3+ tumor-infiltrating lymphocytes were isolated from the tumor single cell suspension using magnetic bead sort with the MojoSort™ Human CD3 T Cell Isolation Kit.

### T Cell and CAF Coculture Assays

25,000 primary CAFs were plated in DMEM supplemented with 10% FBS in 96 well plates. After CAFs had attached to the plate, 50,000 CD3+ T cells were added to the CAFs and cocultured at 37° C. for 5-7 days with or without 20 ng/mL TGFβ. Media was renewed on days 3 and 5. Cocultures with tumor-infiltrating lymphocytes were only cultured for 3 days to preserve TIL viability. Following incubation, T cells were harvested and stained with Live/Dead Aqua (1:1600, Biolegend #423102) for 15 minutes in PBS. Cells were then washed and stained for 15 minutes with an antibody cocktail containing anti-CD4-APC/Fire 810 (1:1000, Biolegend #344661), anti-CD8-BB515 (1:200, BD Biosciences #564526), anti-PD-1-BV421 (1:100, Biolegend #329920), anti-TIM-3-BV786 (1:100, Biolegend #345032), anti-NKG2A-PE (1:200, Biolegend #375104), anti-CD103 (1:400, Biolegend #350212), and anti-CXCR5 (1:100, Biolegend #356928). Cells were then washed, fixed, and permeabilized and stained with an intracellular antibody cocktail containing anti-Perforin-PerCP/Cy5.5 (1:100, Biolegend #353314) and anti-Granzyme B-Alex Fluor 700 (1:100, Biolegend #372222). Cells were subsequently analyzed by flow cytometry using the Cytek Aurora.

### Transwell T Cell and CAF Coculture Assays

100,000 primary CAFs were plated in DMEM supplemented with 10% FBS in the lower chamber of the transwell (0.4 μm pore size, Corning Polycarbonate Membrane Transwells #3401). 200,000 CD3+ T cells were plated in DMEM supplemented with 10% FBS in the upper chamber of the transwell. Cells were incubated at 37° C. for 7 days. Media was renewed on days 3 and 5. Following incubation, T cells were stained and analyzed by flow cytometry using the Cytek Aurora as described above.

### ELISA

The level of IFNγ in cell culture supernatants was measured using an ELISA MAX Deluxe kit (Biolegend #430104) following manufacturer's instructions. Supernatants were collected from CAF-T cell cocultures as described above.

### Statistical Analysis

All quantitative and statistical analyses were performed using the R computational environment and packages described above with the exception of co-culture experiments. Statistical analyses of co-culture assays were performed using Prism 9 software (GraphPad). Differential gene expression was assessed at the single-cell level by the MAST single-cell statistical framework as implemented in Seurat v3, and differential VIPER activity was assessed by t-test, each with Benjamini-Hochberg multiple-testing correction. Comparisons of cell frequencies were performed by non-parametric Wilcox rank-sum test, and survival analyses were performed by log-rank test. In all cases, statistical significance was defined as an adjusted p-value less than 0.05. Details of all statistical tests used can be found in the corresponding figure legends.

### Discussion

In this study, protein activity profiles, as measured by the VIPER algorithm analysis of a longitudinal single-cell transcriptomics HNSCC dataset, was used to identify five molecularly distinct CAF subtypes. The longitudinal approach was used to show that two subtypes, HNCAF-0 and HNCAF-3, are predictive of favorable clinical responses to PD-1 checkpoint blockade therapy. Moreover, it was discovered HNCAF-0 cells have an immunostimulatory effect on CD8 T cells while HNCAF-1 cells are associated with immunosuppression. From a functional perspective, it was shown that HNCAF-0 fibroblasts prevent induction of an exhaustive T Cell phenotype and increase CD-8 T Cell cytotoxicity and tissue localization. Interestingly, in contrast, the presence of HNCAF-1 fibroblasts correlates with increased T cell exhaustion, suggesting contrasting roles for these CAF subtypes.

Immune checkpoint inhibitors (ICI) have revolutionized the field of cancer immunotherapy with monoclonal antibodies targeted against CTLA-4, PD-1, and PD-L1 being recently approved for use as frontline therapies, however, response rates can be as low as 20%. The factors guiding resistance mechanisms to ICI remain largely unknown, making it difficult to predict who will respond and who will not. Accordingly, there remains an unmet need for reliable biomarkers predictive of response to guide patient selection and optimization of ICI treatment. In recent studies, CAFs have been suspected to influence response to checkpoint immunotherapy. A preclinical model of pancreatic ductal adenocarcinoma showed that depletion of CAFs expressing high levels of fibroblast activation protein improves response to αPD-L1 blockade. Similarly, single cell RNA sequencing revealed a CAF population associated with worse response to αPD-L1 immunotherapy in a clinical trial for bladder cancer. Furthermore, distinct CAF populations identified in breast cancer were also shown to be associated with poor αPD-1 immunotherapy response in melanoma and lung cancer. These studies have implicated CAFs as contributors to resistance; however, the repertoire of molecularly distinct CAF subtypes and their role in mediating the effect of immunotherapy remains poorly investigated. This Example shows that the presence of two HNSCC-specific CAF subtypes are predictive of clinical response to immunotherapy. In particular, these findings suggest that HNCAF-0 fibroblasts are active participants in the immune response elicited by PD-1 directed immunotherapy through T cell modulation.

The idea that CAFs may alter T cell behavior is not a new concept, however, previous studies have typically shown CAFs as promoters of immunosuppression. CAFs have been shown to prevent T cell infiltration and to kill T cells in an antigen-dependent manner, via PD-L2 and FasL. CAFs have also been shown to suppress T cells through upregulation of PD-L1 and PD-L2 and through recruitment of regulatory T cells. in contrast, while confirming the immunosuppressive role of some CAFs, this work has established a new pro-inflammatory role for a specific CAFs subtype, which acts as promoters of T cell activation and cytotoxicity. As disclosed herein, HNCAF-0 cells may repress SMAD3 to transcriptionally inhibit PD-1/TIM-3 expression.

Interconversion of CAF subtypes has also been previously demonstrated. This work, however, identifies a new therapeutic opportunity for exploitation of CAF plasticity by forcing CAF differentiation towards the pro-inflammatory HNCAF-0 subtype rather than the HNCAF-1 immunosuppressive one, in combination with immunotherapy. Critically, this study highlights a much greater molecular heterogeneity of CAF subtypes than previously appreciated and demonstrates the critical need to functionally characterize its pleiotropic effects in terms of cancer progression, outcome, and response to immunotherapy and other cancer treatments.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating a solid tumor in a subject, comprising
(a) detecting in a tumor biopsy sample from the subject an enrichment of a cancer associated fibroblast (CAF) subset differentially expressing:
(1) a plurality of genes in a first cluster (Cluster-0) selected from the group consisting of AEBP1, ALPL, ANGPTL2, ANKH, ANKRD28, ANTXR1, APOL2, APP ASPN, B4GALT1, BAMBI, BGN, BICC1, BNIP3, C10orf10, C1orf54, CADM1, CCDC3, CCNG2, CD276, CD9, CDC42EP3, CDH11, CDKN2A, CERCAM, CITED2, CKAP4, CKB, CLEC11A, CLMP CNKSR3, COL12A1, COL16A1, COL1A1, COL1A2, COL3A1, COL5A1, COL5A2, COL6A3, COL8A1, COPZ2, CPE, CSF1, CTGF, CTHRC1, CTNNB1, CTSK, CTSZ, CXCL12, CXCL14, CYR61, DCN, DDIT4, DLX5, EDIL3, EFEMP2, EFNA5, EMILIN1, ENAH, ENPP2, ERRF11, EVI2A, FAM134B, FAP FBLN1, FBLN7, FCGRT FGFR1, FKBP10, FLRT2, FMOD, FNDC3B, FSCN1, FXYD6, GADD45G, GALNT1, GAS1, GGT5, GJA1, GLT8D2, GOLIM4, GOLM1, GPNMB, HAPLN1, HERPUD1, HES1, HSPG2, HTRA1, ID2, IGF2, IGFBP4, IGHG1, IGHG3, IGHG4, IGKC, IGLC2, IRX3, JCHAIN, KCNQ1OT1, KCTD12, KDELR3, LAMB1, LAPTM4A, LAYN, LIMCH1, LMO7, LOXL1, LPAR1, LSP1, LUM, MAFB, MAP4K4, MARCKS, MDK, MFAP2, MIR99AHG, MMP11, MMP14, MMP2, MT1E, MT1X, MT-ND3, MT-ND4, MT-ND5, MXRA5, MXRA8, MZB1, NBL1, NNMT NPC2, NR3C1, NRP2, OLFML2B, OLFML3, P3H4, PALLD, PCOLCE, PDGFC, PDGFD, PDPN, PFN2, PIK3R1, PLD3, PLEKHA5, PODNL1, POSTN, PPA1, PRRX1, PRSS23, PSAP PTPRS, QKI, RAB31, RA114, RCAN1, RCN3, RGS3, S100A10, S100A13, S100A4, SCRG1, SDC1, SDC2, SEPP1, SERPINF1, SERPINH1, SERTAD4, SESN3, SFRP2, SH3BP5, SMOC2, SNA12, SPARC, SRP14, SSPN, SSR4, STK17B, THBS2, TIMP3, TPST1, TSC22D3, UNC5B, VCAM1, VCAN, VIM, WIPF1, YPEL2, ZEB1, ZFHX4, and ZFP36L2; and/or
(2) a plurality of genes in a second cluster (Cluster-3) selected from the group consisting of ACTB, ADAM121, ADAM19, ADAMTS2, AKR1B11, ALDH2, ANPEP ANXA2, ANXA5, ANXA61, APOL1, AQP1, ARID5A2, ARL4C, BASP11, BMP2, BPGM, BST21, C12orf75, CALD11, CCL21, CD2481, CD82, CEP57L1, CHMP1B1, CHPF2, COL15A1, COL1A11, COL3A11, COL5A11, COL5A21, COL5A31, COL6A1, COL6A2, COL6A31, COL7A1, CRABP2, CREB3L21, CTHRC11, CXCL11, CXCL3, CXCL81, DIXDC1, DNAJA11, EIF5A, EMILIN11, ENQ1, EVA1A, F2R1, F3, FAM129B, FAM19A5, FARP11, FKBP11, FN1, FSTL11, FTH11, GAPDH, GBP1, GCNT1, GLIPR2, GLIS31, GNAI2, GOLM11, GPM6B, GREM11, GUCY1A31, H1F01, HES41, HIF1A1, HILPDA, HLA-A1, HLA-B, HLA-C, HSPA181, HSPA5, HSPH1, HTRA3, IFI271, IFI44L1, IFI61, IFIT1, IFIT31, IFITM1, IGFBP3, IL11, IL24, IL32, IL61, INHBA, IRF7, ISG151, ISLR, ITGA5, ITGAV1, KLF6, LAMA41, LGALS1, LGMN1, LINC001521, LOXL11, LOXL2, LY6E, MAGEH1, MEST, METRNL, MFAP21, MFAP51, MME1, MMP1, MMP111, MMP141, MT-C02, MX1, MX21, NES1, NOX4, NREP NTM1, OAS1, PAPPA1, PDLIM4, PFN1, PHLDA31, PKM, PLAT, PLAU, PLXNC1, PMEPA1, POSTN1, PRRX21, PTGES1, PTK7, PXDN, RARRES2, RIN2, RORA1, RP11-115D19.1, RP3-325F22.5, S100A11, S100A16, S100A61, SAT1, SCG5, SCX, SEC23A, SELM, SERINC2, SERPINE1, SERPINH11, SGK1, SPON2, STC2, STEAP11, SUGCT′ SULF1, TAGLN1, TCTN3, TGFBI, TGM2, TIMP2, TMEM158, TMEM45A, TMSB10, TNC, TNFAIP61, TNFRSF12A1, TPM11, TPM21, TRIOBP TUBA1A, TUBA1C, TWIST21, TYMP VCAN1, WARS, WDFY1, WIPI1, WNT5A1, XAF1, ZFAND2A, and ZMPSTE24; and
(b) treating the subject with an immunotherapy.

2. A method for treating a solid tumor in a subject, comprising
(a) detecting in a tumor biopsy sample from the subject a cancer associated fibroblast (CAF) subset expressing:
(1) a plurality of differentially activated proteins in a first cluster (Cluster-0) selected from the group consisting of ABCC9, ACAP1, ACKR1, ADAP2, ADGRL4, ADRA2B, AGT, AK1, AKAP13, AKNA, ALPL, ANKH, ANTXR1, ANXA1, ANXA4, APBB1IP, APOLD1, APP, ARF4, ARF5, ARHGAP15, ARHGAP30, ARHGEF19, ARID4B, ARID5B, ARL2BP, ARL4A, ARL4C, ARRDC2, ASH1L, ATF6B, ATP2B1, ATP6AP2, ATRAID, AXL, BASP1, BATF, BCL11B, BCL2L11, BTG1, BTG2, C18orf32, C2orf88, C3AR1, CADM1, CALML5, CAMLG, CAPN2, CAPNS2, CAPS, CBLB, CCL4L2, CCL5, CCNH, CCR6, CCR7, CD1C, CD2, CD24, CD247, CD27, CD3D, CD3E, CD3G, CD48, CD5, CD53, CD7, CD74, CD8A, CD8B, CD9, CD96, CD99, CDC42BPA, CDC42EP3, CDH11, CDKN2A, CDKN2B, CEACAM6, CEBPB, CERCAM, CFB, CHD9, CLEC10A, CLEC4A, CLMP, CLU, CNIH1, CNKSR3, COLEC12, CORO1A, CPE, CPM, CPNE7, CRABP2, CREB3L1, CREB3L2, CREM, CRYAB, CSDE1, CSF2RA, CTLA4, CTNNB1, CTSH, CTSZ, CXCR3, CXCR4, CXCR6, CYBRD1, DAP, DAPP1, DDAH2, DDIT4, DDR2, DDX5, DERL3, DLX5, DNAJB6, DPYSL3, DSG1, DST, DTHD1, DUSP1, DUSP2, DUSP4, EBF1, EEF1D, EID1, EIF5, ELF1, EMP1, EMP2, EMP3, ENAH, ENPP2, EPB41, EPB41L2, EPHB2, ESD, ETS1, ETV3, EVI2A, EVL, EZR, F2RL3, FAP, FBXW7, FCER1A, FCER1G, FCGR2B, FGF7, FGFR1, FGFR2, FHL1, FNBP1, FNDC4, FOSL2, FOXC1, FOXC2, FOXO3, FPR1, FXYD6, FYN, FZD1, GAS1, GATA2, GDI2, GJA1, GLIPR1, GNAS, GNG7, GPBP1, GPC1, GPR132, GPR157, GPR171, GPR183, GPR65, GRAP2, GRIN2A, GSN, GSPT1, GZMA, GZMB, HCST, HDAC7, HDLBP, HERPUD1, HEXIM1, HLA-B, HLA-DOA, HLA-DOB, HLA-DPA1, HLA-DPB1, HLA-DQA1, HLA-DQB1, HLA-DRA, HLA-DRB1, HLA-DRB5, HLA-E, HNRNPDL, HOXB2, HPGD, HSPG2, HYAL2, ICOS, ID2, ID3, IFI16, IFITM2, IFNG, IKZF1, IKZF3, IL10RA, IL2RA, IL2RB, IL2RG, IL6ST, INPP5D, INTS6, ITGA10, ITGA11, ITGA4, ITGB1BP1, ITGB2, ITGB5, ITGBL1, ITM2B, ITM2C, JADE1, JAML, JMJD1C, JUN, JUNB, KCNMA1, KIR2DL4, KLF2, KLF9, KLRB1, KLRC1, KLRC2, KLRD1, LAX1, LBH, LCK, LCP1, LGALS3, LIFR, LPAR2, LPL, LRP1, LSP1, LTB, LY86, LY96, MAFB, MAGED1, MAPK13, MARCO, MARVELD1, MEF2C, MEOX2, MGST3, MKX, MMP2, MORF4L1, MS4A6A, MTUS1, MYADM, MYC, NDFIP1, NDN, NDRG2, NEO1, NET1, NFIX, NOTCH2, NOTCH4, NR3C1, NR4A3, NRP2, NSG1, NTRK3, NUDT4, PALLD, PASK, PBX1, PDCD1, PDCD4, PDE4B, PDGFC, PDGFRA, PEBP1, PFDN5, PIK3IP1, PIK3R1, PINK1, PITX1, PLEKHO1, PLSCR4, PNRC1, PPIC, PPP1R10, PPP1R2, PPP2R5C, PRDX4, PRF1, PRMT2, PRRX1, PRSS27, PSCA, PSIP1, PTGIS, PTH1R, PTN, PTP4A2, PTPRC, PTPRD, RAB30, RAB31, RAB32, RAB33A, RABAC1, RAMP2, RAMP3, RAP1B, RASGEF1B, RASGRP1, RASSF4, RASSF5, RASSF7, RBM38, RBPJ, REL, RERG, RGL4, RGS1, RGS10, RGS3, RHOD, RHOF, RHOH, RHOJ, RIPK2, RORA, RPS27L, RPSA, RRBP1, RUNX2, RUNX3, S100A10, S100A6, S1PR3, S1PR4, SAP18, SDC1, SDC2, SDCBP, SELP, SEMA7A, SERBP1, SFPQ, SH2D1A, SH2D2A, SH3BGRL, SH3BP5, SH3KBP1, SIT1, SKAP1, SKAP2, SKIL, SLA, SLA2, SLC29A1, SLC38A5, SLC41A2, SLC44A1, SLC7A5, SLCO2A1, SMAP2, SMOC1, SMOC2, SOCS1, SOD1, SOX18, SP100, SPIB, SPOCK2, SPRED1, SRSF2, STAB1, STEAP4, STK17B, STK4, STX11, STXBP6, SVIP, SYTL3, TANK, TBC1D10C, TCEA3, TCEAL2, TCEAL4, TERF2IP, TGFB1I1, TGFBI, THY1, TLE4, TMEM204, TMEM59, TNFAIP8L3, TNFRSF18, TNFSF12, TOB1, TRAF1, TRAT1, TRIM22, TSC22D3, TSHZ2, TSPAN4, TSPO, TWIST1, TXNIP, TYROBP, UBC, UBE2B, UNC5C, UTRN, VAMP2, VASN, VASP, VCAM1, VOPP1, VPS37B, WASF2, WSB1, WWTR1, YWHAQ, ZBTB16, ZBTB20, ZEB1, ZFAND5, ZFHX3, ZFHX4, ZFP36L2, ZNF106, ZNF296, and ZNF428; and/or
(2) a plurality of differentially activated proteins in a second cluster (Cluster-3) selected from the group consisting of ABL2, ACAP11, ACHE1, ACKR3, ACKR41, ACTB1, ACTG1, ACTN11, ADAM12, ADD3, ADM1, ADRB21, AES1, AGPAT21, AGTRAP1, AHNAK21, AKAP131, ANGPT21, ANGPTL41, ANKRD112, ANKRD12, ANTXR11, ANXA5, AP2B11, AP2M1, AP2S1, AP3S1, APBA2, APOE1, AQP91, ARC1, ARF11, ARF41, ARHGAP152, ARNTL21, ARPC21, ASH1L1, ATP1B1, ATP2B11, ATP6AP21, ATP6V1G11, AVPR1A1, AXL1, B2M1, B4GALT1, BASP11, BATF1, BATF3, BAX1, BDKRB11, BHLHE401, BIRC31, BMP21, BSG1, BST21, BZW11, C18orf321, C31, C5AR11, CALD11, CALM2, CALM3, CALR1, CAP11, CASP11, CAV11, CAV21, CBFA2T31, CBX31, CCL21, CCL4L21, CCL8, CCR62, CCRL21, CD1641, CD1771, CD1E1, CD2741, CD300E1, CD402, CD441, CD471, CD51, CD591, CD63, CD79A2, CD82, CD831, CDC421, CDC42SE1, CEMIP, CERCAM1, CFL11, CFLAR, CHD31, CHIC2, CHMP4A, CHMP51, CHN11, CHP1, CHST11, CKS2, CLEC2B1, CLEC5A, CLEC7A1, CLIC11, CLIC4, CNIH41, CPNE11, CRABP21, CREB3L11, CSF2RA1, CSF2RB1, CSNK2B2, CTNNAL11, CXCL101, CXCL21, CXCL3, CYBA, DDR21, DIO21, DLL11, DNAJB61, DPP4, DRAP11, DUSP61, ECM1, EDF1, EDNRA1, EDNRB1, EFNA1, EHD11, EID3, EMP11, ENG1, ENO11, ENTPD11, ENY2, EREG1, ERO1A1, ETS22, ETV31, EVA1A, F2R, F2RL21, F2RL31, F31, FAP1, FCER1A2, FCER1G1, FCGR1B2, FKBP8, FLNA1, FLT31, FOSL21, FOXP1, FOXS11, FPR12, FRMD61, FST1, FSTL11, GABARAP, GAPDH1, GEM1, GGT51, GLIPR11, GNAI1, GNB11, GNG11, GNG2, GNG51, GPBP11, GPM6B1, GPR1571, GPR1831, GPX11, GREM1, GRK5, GRN1, GYPC1, HBEGF1, HCAR22, HCAR32, HES42, HIF1A, HINT11, HIVEP3, HLA-A1, HLA-B1, HLA-C1, HLA-F1, HM13, HPGD1, HRAS, HSBP11, HSPA51, ICAM11, IFI271, IFI61, IFITM11, IFITM21, IFITM31, IFNGR11, IGF2, IGFBP31, IGFBP61, IGFLR11, IL10, IL11, IL15RA1, IL1A1, IL1B1, IL1R1, IL1R22, IL1RAP1, IL2RA1, IL2RG1, IL61, IL7R, ILK1, INHBA, INSIG11, IRF41, IRF7, ITGA1, ITGA5, ITGAV, ITGB1, ITGB42, ITGB61, ITSN22, KCNJ81, KCNK61, KIR2DL41, KLF61, KLRB11, KLRC11, LAMP5, LCP21, LGALS1, LGALS3BP1, LGALS9, LHX81, LILRA11, LILRB21, LIMA11, LIMS11, LMCD1, LMO41, LOXL2, LPXN, LRRFIP11, LSR2, LTB1, LY6E1, LY6K, LYPD11, MAP1B1, MAP3K81, MARCKS1, MARCKSL1, MGST21, MIF1, MMP14, MORF4L2, MSC, MSN1, MSX2, MX1, MXD11, MYADM2, MYH9, MYO101, MYO1G1, NACC1, NAMPT1, NCOA71, NDUFA131, NEDD41, NEDD81, NEO11, NET12, NFE2L3, NFKB11, NGFR1, NLRP31, NME21, NOTCH31, NRG11, NRP11, NTM1, OLR11, PAG1, PALLD1, PARK72, PARP14, PDGFRB1, PDIA31, PDIA61, PDLIM11, PDPN, PFDN51, PFN11, PHLDA1, PILRA1, PIM21, PKIG1, PKM1, PLAT1, PLAU, PLAUR, PLEC2, PLEK1, PLK2, PLP22, PLPP32, PLPP4, PLSCR11, PLXDC11, PMAIP1, PMEPA1, POLR2L1, PON2, PPIC1, PPP1R21, PRCP1, PRDM1, PRDX41, PRKAR1A1, PRMT11, PROCR2, PRRX11, PRRX2, PSMA4, PTEN, PTGER3, PTGES, PTGIR, PTHLH, PTK7, PTPN11, PTPRE1, PTTG11, RAB101, RAB131, RAB1A, RAB301, RAB311, RAB321, RAB33A1, RAB5C1, RABAC11, RANBP11, RAP1A, RAP1B1, RASD1, RASGEF1B1, RBPJ1, RBPMS, REL2, RGL41, RGS161, RGS2, RGS31, RGS4, RHEB2, RHOBTB11, RHOC, RHOF1, RHOG2, RHOH1, RIN2, RIPK21, RND31, RPS27L1, RRAD1, RRBP11, S100A111, S100A121, S100A16, S100A61, SCAND11, SCG5, SDC11, SDC41, SECTM12, SELE1, SERPINB9, SERPINE12, SGIP11, SGK11, SHISA51, SIGLEC102, SKIL1, SLA21, SLC1A51, SLC24A41, SLC2A31, SLC2A6, SLC39A14, SLC3A21, SLC41A21, SLC7A111, SLC7A51, SLC9A3R22, SNA121, SOD21, SOX11, SOX4, SQSTM11, STAT11, STAT2, STEAP1, STEAP21, STX111, SUB11, SULF1, SULF21, SYTL21, TANK1, TAX1BP31, TCF41, TFP11, TGFB1I11, TGFB31, TGIF1, THBD1, THBS11, THY11, TLR21, TMEM2041, TNF1, TNFAIP31, TNFAIP61, TNFRSF12A1, TNFRSF1A, TNFRSF1B1, TNFRSF21, TNFSF101, TNFSF13B2, TNFSF141, TRAF11, TREM11, TRIB11, TRIM221, TSC22D11, TSPAN151, TSPAN9, TWIST11, TWIST2, TXNDC171, UACA1, UBB1, UBE2B1, UBE2D31, UBE2L31, VAMP21, VAMP51, VAMP82, VASP2, VCAM11, VDAC11, VEGFA1, WNT2, WNT5A, XBP11, YWHAH1, ZNF2671, ZNF2962, ZNF469, ZNF503, and ZNHIT11; and
(b) treating the subject with an immunotherapy.

3. The method of claim 1, wherein the immunotherapy is a T cell immunotherapy.

4. The method of claim 3, wherein the T cell immunotherapy comprises a chimeric antigen receptor (CAR) T-cell therapy or tumor-infiltrating lymphocyte (TIL) therapy.

5. The method of claim 1, wherein the immunotherapy comprises a checkpoint inhibitor.

6. The method of claim 5, wherein the checkpoint inhibitor comprises an anti-PD-1 antibody, anti-PD-L1 antibody, anti-CTLA-4 antibody, or a combination thereof.

7. The method of claim 1, wherein the solid tumor comprises a sarcoma, carcinoma, or lymphoma.

8. The method of claim 1, wherein the CAF subset comprises at least 30% of the fibroblasts in the tumor biopsy.

9. The method of claim 1, wherein the CAF subset is detected by detecting differential expression of at least five (5) genes in the first cluster and at least five (5) genes in the second cluster.

10. The method of claim 1, wherein the CAF subset comprises at least 30% of the fibroblasts in the tumor biopsy.

11. A method for treating a solid tumor in a subject, comprising
(a) isolating cancer associated fibroblasts (CAFs) from the subject,
(b) isolating and expanding a subset of the CAFs that differentially express:
(1) a plurality of genes in a first cluster (Cluster-0) selected from the group consisting of AEBP1, ALPL, ANGPTL2, ANKH, ANKRD28, ANTXR1, APOL2, APP ASPN, B4GALT1, BAMBI, BGN, BICC1, BNIP3, C10orf10, C1orf54, CADM1, CCDC3, CCNG2, CD276, CD9, CDC42EP3, CDH11, CDKN2A, CERCAM, CITED2, CKAP4, CKB, CLEC11A, CLMP CNKSR3, COL12A1, COL16A1, COL1A1, COL1A2, COL3A1, COL5A1, COL5A2, COL6A3, COL8A1, COPZ2, CPE, CSF1, CTGF, CTHRC1, CTNNB1, CTSK, CTSZ, CXCL12, CXCL14, CYR61, DCN, DDIT4, DLX5, EDIL3, EFEMP2, EFNA5, EMILIN1, ENAH, ENPP2, ERRF1, EVI2A, FAM134B, FAP FBLN1, FBLN7, FCGRT FGFR1, FKBP10, FLRT2, FMOD, FNDC3B, FSCN1, FXYD6, GADD45G, GALNT1, GAS1, GGT5, GJA1, GLT8D2, GOLIM4, GOLM1, GPNMB, HAPLN1, HERPUD1, HES1, HSPG2, HTRA1, ID2, IGF2, IGFBP4, IGHG1, IGHG3, IGHG4, IGKC, IGLC2, IRX3, JCHAIN, KCNQ1OT1, KCTD12, KDELR3, LAMB1, LAPTM4A, LAYN, LIMCH1, LMO7, LOXL1, LPAR1, LSP1, LUM, MAFB, MAP4K4, MARCKS, MDK, MFAP2, MIR99AHG, MMP11, MMP14, MMP2, MT1E, MT1X, MT-ND3, MT-ND4, MT-ND5, MXRA5, MXRA8, MZB1, NBL1, NNMT NPC2, NR3C1, NRP2, OLFML2B, OLFML3, P3H4, PALLD, PCOLCE, PDGFC, PDGFD, PDPN, PFN2, PIK3R1, PLD3, PLEKHA5, PODNL1, POSTN, PPA1, PRRX1, PRSS23, PSAP PTPRS, QKI, RAB31, RA114, RCAN1, RCN3, RGS3, S100A10, S100A13, S100A4, SCRG1, SDC1, SDC2, SEPP1, SERPINF1, SERPINH1, SERTAD4, SESN3, SFRP2, SH3BP5, SMOC2, SNA12, SPARC, SRP14, SSPN, SSR4, STK17B, THBS2, TIMP3, TPST1, TSC22D3, UNC5B, VCAM1, VCAN, VIM, WIPF1, YPEL2, ZEB1, ZFHX4, and ZFP36L2; and/or
(2) a plurality of genes in a second cluster (Cluster-3) selected from the group consisting of ACTB, ADAM121, ADAM19, ADAMTS2, AKR1B11, ALDH2, ANPEP ANXA2, ANXA5, ANXA61, APOL1, AQP1, ARID5A2, ARL4C, BASP11, BMP2, BPGM, BST21, C12orf75, CALD11, CCL21, CD2481, CD82, CEP57L1, CHMP1B1, CHPF2, COL15A1, COL1A11, COL3A11, COL5A11, COL5A21, COL5A31, COL6A1, COL6A2, COL6A31, COL7A1, CRABP2, CREB3L21, CTHRC11, CXCL11, CXCL3, CXCL81, DIXDC1, DNAJA11, EIF5A, EMILIN11, ENO1, EVA1A, F2R1, F3, FAM129B, FAM19A5, FARP11, FKBP11, FN1, FSTL11, FTH11, GAPDH, GBP1, GCNT1, GLIPR2, GLIS31, GNAI2, GOLM11, GPM6B, GREM11, GUCY1A31, H1F01, HES41, HIF1A1, HILPDA, HLA-A1, HLA-B, HLA-C, HSPA181, HSPA5, HSPH1, HTRA3, IFI271, IFI44L1, IFI61, IFIT1, IFIT31, IFITM1, IGFBP3, IL11, IL24, IL32, IL61, INHBA, IRF7, ISG151, ISLR, ITGA5, ITGAV1, KLF6, LAMA41, LGALS1, LGMN1, LINC001521, LOXL11, LOXL2, LY6E, MAGEH1, MEST METRNL, MFAP21, MFAP51, MME1, MMP1, MMP111, MMP141, MT-C02, MX1, MX21, NES1, NOX4, NREP, NTM1, OAS1, PAPPA1, PDLIM4, PFN1, PHLDA31, PKM, PLAT PLAU, PLXNC1, PMEPA1, POSTN1, PRRX21, PTGES1, PTK7, PXDN, RARRES2, RIN2, RORA1, RP11-115D19.1, RP3-325F22.5, S100A11, S100A16, S100A61, SAT1, SCG5, SCX, SEC23A, SELM, SERINC2, SERPINE1, SERPINH11, SGK1, SPON2, STC2, STEAP11, SUGCT SULF1, TAGLN1, TCTN3, TGFBI, TGM2, TIMP2, TMEM158, TMEM45A, TMSB10, TNC, TNFAIP61, TNFRSF12A1, TPM11, TPM21, TRIOBP TUBA1A, TUBA1C, TWIST21, TYMP VCAN1, WARS, WDFY1, WIPI11, WNT5A1, XAF1, ZFAND2A, and ZMPSTE24; and
(c) administering the expanded CAF subset to the subject in combination with an immunotherapy.

12. The method of claim 11, wherein the immunotherapy is a T cell immunotherapy.

13. The method of claim 12, wherein the T cell immunotherapy comprises a chimeric antigen receptor (CAR) T-cell therapy or tumor-infiltrating lymphocyte (TIL) therapy.

14. The method of claim 11, wherein the immunotherapy comprises a checkpoint inhibitor.

15. The method of claim 14, wherein the checkpoint inhibitor comprises an anti-PD-1 antibody, anti-PD-L1 antibody, anti-CTLA-4 antibody, or a combination thereof.

16. The method of claim 11, wherein the solid tumor comprises a sarcoma, carcinoma, or lymphoma.

17. The method of claim 11, wherein the CAF subset comprises at least 30% of the fibroblasts in the tumor biopsy.

18. The method of claim 11, wherein the CAF subset is detected by detecting differential expression of at least five (5) genes in the first cluster and at least five (5) genes in the second cluster.
